(19) United States
(12) Patent Application Publication
(10) Pub. No.: US 2022/0035892 A1
(43) Pub. Date: Feb. 3, 2022

(54) STATISTICAL ANALYSIS SYSTEM AND STATISTICAL ANALYSIS METHOD USING CONVERSATIONAL INTERFACE

(71) Applicant: YOOJINBIOSOFT CO., LTD., Goyang (KR)

(72) Inventors: Jin Tae YOU, Seoul (KR); Jin Ho YOO, Goyang (KR)

(21) Appl. No.: 17/276,671

(22) PCT Filed: Sep. 17, 2019
(86) PCT No.: PCT/KR2019/012005

§ 371 Date: Mar. 16, 2021

(30) Foreign Application Priority Data
Sep. 17, 2018 (KR) 10-2018-0110900

(51) Int. Cl. G06F 17/18; G06F 16/23; G06F 40/30; G06F 40/40

(52) U.S. Cl. CPC G06F 17/18; G06F 40/40; G06F 40/30; G06F 16/23

(57) ABSTRACT

The present disclosure relates to a statistical analysis system. More particularly, the present disclosure relates to a statistical analysis system capable of inferring the purpose of a user's analysis, etc. through questions and answers with the user so that ordinary people easily acquire clinical and statistical analysis information, the system using a conversational interface adapted to a statistical analysis for clinical data. The present disclosure provides a statistical analysis system using a conversational interface adaptive to a statistical analysis method using clinical data, and the statistical analysis method using the system, wherein a conversational interface is applied to extract variable characteristic information required for a statistical analysis according to the purpose of the statistical analysis that a user wants and to select and set a statistical analysis algorithm according to the extracted information so that the statistical analysis is performed and statistical analysis data that the user wants is generated.

### TECHNICAL FIELD

The present disclosure relates to a statistical analysis system. More particularly, the present disclosure relates to a statistical analysis system capable of inferring the purpose of a user's analysis, etc. through questions and answers with the user so that ordinary people can easily acquire clinical and statistical analysis information, the system using a conversational interface adapted to a statistical analysis for clinical data.

### BACKGROUND ART

As multimedia and semiconductor technologies have been developed, statistical analysis programs have been used in various fields where enormous amounts of data, etc. are processed into information and the information is statistically organized whereby a result thereof is provided, inference about new information and access to new facts are easy, and so on.

In particular, in clinical fields, software equipped with various statistical analysis algorithms is frequently used to find factors that cause diseases or that is related to occurrence of diseases, and to analyze the effects of newly developed drugs or treatments.

A liver somatic index, a cholesterol level, blood pressure, body mass index (BMI), information on whether a person smokes or not, etc. are clinical and epidemiological variables that are typically acquired in hospitals.

These clinical and epidemiological variables may expand to several tens or more because of variables acquired through measurement, observation, or experiment depending on the purpose of treatment or research.

In particular, there may be various associations between the clinical and epidemiological variables. In addition, as newly developed medicines, treatments, checkups, etc. have been diversified with the development of modern medical science, variables have been generated or have increased constantly. The association that may exist between the variables is inevitably complicated.

Software only for a statistical analysis is equipped with various statistical analysis algorithms that consider the association which may exist between the clinical and epidemiological variables in analyzing, thereby constructing a statistical analysis process.

The most representative software only for a statistical analysis equipped with various statistical analysis algorithms is SAS or SPSS, which is the software only for a statistical analysis that most researchers use currently.

The analysis software derives an accurate result under the premise that the user clearly knows variables to be analyzed and statistical algorithms to be applied, but it is somewhat difficult for ordinary people who relatively lack statistical knowledge and even for clinicians or researchers to use.

For example, assuming that there are 10 variables to be analyzed, when considering the case of only three statistical algorithms to be applied, the number of available analysis methods for the variables is 135 (the number of available variable combinations of two variables x the number of algorithms=10×9/2×3). Therefore, it is practically difficult for non-statisticians to use a statistical analysis software for an analysis.

FIGS. 1 to 3 are diagrams illustrating an example of a method of performing a statistical analysis by using a conventional statistical analysis program, which is an example using IBM SPSS.

FIGS. 1 to 3 illustrate an example of a mean difference analysis of continuous variables between two different groups.

As shown in FIG. 1, as “mean comparison” is chosen from a main menu, a sub menu of statistical algorithms that a user can choose is displayed.

When the user chooses “independent sample T test” from the sub menu, a menu for choosing a continuous variable and a group variable for mean comparison is displayed as shown in FIG. 2.

The user chooses a continuous variable and a group variable accordingly as shown in FIG. 2.

Through these steps, a statistical analysis is performed, so that a result of the statistical analysis may be acquired as shown in FIG. 3. Like (a), the entire result may be chosen for use. Alternatively, like (b) and (c), only required content may be chosen from the result of the statistical analysis.

Result (b) is a condition used for result (c) selection, and result (c) is a final result candidate.

FIG. 4 illustrates another example in which a method of selecting a statistical algorithm is used for a mean difference analysis of a continuous variable.

As described above, in the use of the conventional statistical analysis program, for a statistical analysis, the user manually selects an individual statistical analysis method and manually selects a particular algorithm, variables, and other analysis parameters that are used for the analysis.

Therefore, it is very difficult for a user who does not know statistical analysis methods or particular statistical algorithms to use the statistical analysis program. Even statisticians who well know statistical analysis methods or particular statistical algorithms have difficulty in using the program if they are not familiar with the operation method of the program and the analysis result format.

Accordingly, the applicant invented Korean Patent Application No. 10-2011-0104734, “METHOD OF AUTOMATICALLY EXPLORING ASSOCIATIONS BETWEEN VARIABLES AND GENERATING DYNAMIC RESULT REPORT BY USING SAME”.

In this conventional invention, most analysis parameters frequently used in a statistical analysis are predetermined. In addition, the conventional invention is based on the following idea: a statistical algorithm is automatically selected according to the variable characteristics, even for a complicated analysis performed by statisticians, considering the process of the analysis in details, wherein the process is converted into a process of selecting two or more conditions, which may be provided as propositions, through several steps.

The invention of the applicant has technical features that the characteristics of a number of clinical and epidemiological variables used in medical statistics are identified for automatic classification into the most frequently used type and when there are many variables to be analyzed and statistical algorithms to be applied, every association between all the variables is automatically designated.

The conventional method of automatically exploring associations between variables is to separate types of clinical and epidemiological variables considering the characteristics thereof according to a process determined in a statistical algorithm and to determine a statistical algorithm to be automatically applied, accordingly. A statistical algorithm is automatically applied according to the variable characteristics, so that even users who do not know statistical analysis methods or particular statistical algorithms well may easily use a statistical analysis program. However, it is not guaranteed that an appropriate statistical analysis algorithm matched to the purpose of the statistical analysis that the user wants to proceed is selected and a statistical analysis is performed.

### DISCLOSURE

### Technical Problem

To achieve an effective statistical analysis, an appropriate statistical analysis program needs to be selected considering the purpose of use of the program, the characteristic according to the corresponding field, etc.

The present disclosure is directed to providing a statistical analysis system using a conversational interface adaptive to a statistical analysis method using clinical data, and the statistical analysis method using the system, wherein in order to extract variable characteristic information required for a statistical analysis and automatically select a statistical algorithm required for the statistical analysis according to the extracted variable characteristic information, a conversational interface is applied to extract variable characteristic information required for a statistical analysis according to the purpose of the statistical analysis that a user wants and to select and set a statistical analysis algorithm according to the extracted information so that the statistical analysis is performed and statistical analysis data that the user wants is generated.

### Technical Solution

The present disclosure is directed to providing a statistical analysis system using a conversational interface, the system enabling a statistical analysis of clinical information by selecting and setting an adaptive algorithm according to variable characteristics to be determined, the system having the following technical features:

(a) Through a conversational interface, a user's purpose of a statistical analysis and variable characteristic information required for the statistical analysis according to the purpose are extracted, and

(b) A required algorithm is set accordingly and statistical analysis data is generated accordingly, so that the statistical analysis system using the conversational interface is adaptive to a statistical analysis for clinical data in which various variables are used.

The present disclosure provides a statistical analysis system using a conversational interface, the system including: a conversational interface means for providing inquiry information to a user to extract a user's purpose of a statistical analysis and variable information, and for collecting answer information provided from the user to inquiries; an analysis feature information extraction means 20 for extracting analysis feature information by using the answer information of the user acquired by the conversational interface means; an algorithm management means for storing and managing statistical algorithms therein and for providing the statistical algorithms according to a request from a statistical analysis control means; the statistical analysis control means for controlling execution of the statistical analysis in such a manner that the conversational interface is provided to the user through the conversational interface means, the analysis feature information is extracted through the analysis feature information extraction means from the answer information collected through the conversational interface, and the statistical algorithm to be used in the statistical analysis is selected according to the analysis feature information; and a statistical analysis means for performing the statistical analysis according to the statistical algorithm set by the statistical analysis control means and for providing information about a result thereof.

In addition, a statistical analysis method using the statistical analysis system using the conversational interface according to the present disclosure includes:

A feature information extraction induction stage in which when a statistical analysis program starts, the conversational interface for conversation with a user is provided so that inquiries are made to the user and answer information to the inquiries is acquired and stored; an analysis feature information extraction stage in which after the feature information extraction induction stage, analysis feature information including a purpose of a statistical analysis that the user wants and variable characteristic information to be applied to the statistical analysis is extracted from the answer information; an algorithm setting stage in which the algorithm matched to the analysis feature information extracted at the analysis feature information extraction stage is set as a statistical analysis algorithm; a statistical analysis execution stage in which a statistical analysis is performed according to the algorithm set through the algorithm setting stage; and a statistical analysis result providing stage in which the user is provided with results acquired through the statistical analysis execution stage.

The feature information extraction induction stage may include: a purpose inquiry stage in which when a statistical analysis system starts, a request to start a conversation analysis is made, and when a request to start the conversation analysis is made from the user, the user is provided with an inquiry for acquiring a purpose of use of the program and an answer thereto is input; and a variable characteristic information inquiry stage in which when the answer is input through the purpose inquiry stage, the answer is stored in an analysis feature information extraction means, inquiry information is provided to create a variable characteristic table corresponding to the user's selected purpose of the statistical analysis, and the answer information thereto is input and stored as the analysis feature information.

### Advantageous Effects

According to the statistical analysis system using the conversational interface of the present disclosure, even if the user doesn't know a specific statistical algorithm, the user can acquire a result of a statistical analysis that the user wants through the process of selecting an item that is matched to the purpose of the analysis through the conversational interface. In particular, the statistical analysis method that is effective in processing clinical data is provided.

### DESCRIPTION OF DRAWINGS

FIGS. 1 to 3 are diagrams illustrating an example of a method of performing a statistical analysis by using a conventional statistical analysis program, specifically, an example of IBM SPSS for a mean difference analysis of continuous variables between two different groups.

FIG. 4 is a diagram illustrating another example in which a method of selecting a statistical algorithm is used for a mean difference analysis of a continuous variable.

FIG. 5 is a block diagram illustrating a configuration of a statistical analysis system using a conversational interface according to the present disclosure.

FIGS. 6 to 37 are diagrams illustrating an example of a process of performing a clinical and statistical analysis method using a conversational interface according to the present disclosure.

FIG. 6 is a flowchart illustrating a whole process of a statistical analysis method using clinical data.

FIG. 7 is a diagram illustrating an example of a conversational interface including inquiry information provided to a user to acquire the purpose of a statistical analysis.

FIG. 8 is a diagram illustrating a conversational interface displaying inquiry information for a statistical analysis for clinical data selected in a purpose inquiry stage.

FIG. 9 is a diagram illustrating a conversational interface for selecting variable characteristics that are a combination of multiple conditions with respect to an analysis of a mean difference between groups of continuous variables (Part 4-1).

FIG. 10 is a diagram illustrating a conversational interface for providing selection information for a statistical algorithm selected with respect to an analysis of a mean difference between groups of continuous variables.

FIG. 11 is a flowchart illustrating a whole process for selecting a statistical algorithm according to variable characteristics in variable characteristic information selected with respect to the purpose of use of a statistical analysis program.

FIGS. 12 to 19 are flowcharts illustrating a method of automatically selecting a particular algorithm, in each algorithm of FIG. 11.

FIG. 20 is a flowchart illustrating a normal distribution test method A for variable data, in the method of automatically selecting a particular algorithm of FIGS. 12 to 19.

FIGS. 21 to 27 are diagrams illustrating a stage of setting, by a user, variables and parameters for each statistical algorithm through an interface.

FIG. 28 is a diagram illustrating an example of a user interface for performing a stage of selecting a group variable for mean comparison, in a stage of setting variables and parameters.

FIG. 29 is a diagram illustrating an example of a user interface for performing selection of a group stratification variable, in a stage of setting variables and parameters.

FIG. 30 is a diagram illustrating an example of a user interface for performing selection of a continuous response variable, in a stage of setting variables and parameters.

FIG. 31 is a diagram illustrating an example of a user interface for performing selection of a continuous numerical value expression method, in a stage of setting variables and parameters.

FIG. 32 is a diagram illustrating an example of a user interface for performing selection of a particular algorithm, in a stage of setting variables and parameters.

FIG. 33 is a diagram illustrating an example of a user interface for performing selection of a result presentation method, in a stage of setting variables and parameters.

FIG. 34 is a diagram illustrating an example of a table editor, in a user interface for performing selection of a result presentation method.

FIG. 35 is a diagram illustrating an example of a figure editor, in a user interface for performing selection of a result presentation method.

FIG. 36 is a diagram illustrating statistical analysis result information, wherein FIG. 36 illustrates an integrated analysis result table, and FIG. 37 is a diagram illustrating a graph that expresses an individual variable analysis result.

FIGS. 38 to 44 are diagrams illustrating a process of a case in which an example (an analysis of a factor influencing a continuous response variable (Part 4-2)) of a process of performing a clinical and statistical analysis method using a conversational interface according to the present disclosure is selected as answer information.

FIG. 38 is a diagram illustrating a conversational interface for selecting variable characteristics that are a combination of multiple conditions with respect to an analysis (Part 4-2) of a factor influencing a continuous response variable.

FIG. 39 is a diagram illustrating a conversational interface for providing selection information for a statistical algorithm selected with respect to an analysis (Part 4-2) of a factor influencing a continuous response variable.

FIG. 40 is a flowchart illustrating a whole process for selecting a statistical algorithm according to variable characteristics (Part 4-2) in variable characteristic information selected with respect to the purpose of use of a statistical analysis program.

FIG. 41 is a flowchart illustrating a stage of automatically selecting a particular algorithm of algorithm “2-way ANOVA”.

FIGS. 42 to 44 are diagrams illustrating a stage of setting, by a user, variables and parameters for each statistical algorithm through an interface.

FIG. 42 illustrates a stage of setting variables and parameters in the cases of “univariable linear regression” and “multivariable linear regression”.

FIG. 43 illustrates a stage of setting variables and parameters in the case of “2-way ANOVA”.

FIG. 44 illustrates a stage of setting variables and parameters in the case of “linear mixed effect model analysis”.

FIGS. 45 to 54 are diagrams illustrating a process of a case in which an example (an analysis of association between categorical variables (Part 4-3)) of a process of performing a clinical and statistical analysis method using a conversational interface according to the present disclosure is selected as answer information.

FIG. 45 is a diagram illustrating a conversational interface for selecting variable characteristics that are a combination of multiple conditions with respect to an analysis of association between categorical variables (Part 4-3).

FIG. 46 is a diagram illustrating a conversational interface for providing selection information for a statistical algorithm selected with respect to an analysis of association between categorical variables (Part 4-3).

FIG. 47 is a flowchart illustrating a whole process for selecting a statistical algorithm according to variable characteristics (Part 4-3) in variable characteristic information selected with respect to the purpose of use of a statistical analysis program.

FIG. 48 is a flowchart illustrating a stage of automatically selecting a particular algorithm of statistical algorithm one-sample proportion test.

FIG. 49 is a flowchart illustrating a stage of automatically selecting a particular algorithm of statistical algorithms Chi-squared test, Yates's correction, and Fisher's exact test.

FIGS. 50 to 54 are diagrams illustrating a stage of setting, by a user, variables and parameters for each statistical algorithm through an interface.

FIG. 50 illustrates a stage of setting variables and parameters in the case of Proportion test.

FIG. 51 illustrates a stage of setting variables and parameters in the cases of Chi-squared test, and Fisher's exact test.

FIG. 52 illustrates a stage of setting variables and parameters in the case of linear-by-linear association.

FIG. 53 illustrates a stage of setting variables and parameters in the cases of McNemar's test, and McNemar-Bowker test.

FIG. 54 illustrates a stage of setting variables and parameters in the cases of Cochran's Q test and Friedman test.

FIGS. 55 to 61 are diagrams illustrating a process of a case in which an example (Part 4-4; a factor analysis and development of a predictive model for categorical response prediction) of a process of performing a clinical and statistical analysis method using a conversational interface according to the present disclosure is selected as answer information.

FIG. 55 is a diagram illustrating a conversational interface for selecting variable characteristics that are a combination of multiple conditions with respect to a factor analysis and development of a predictive model for categorical response prediction (Part 4-4).

FIG. 56 is a diagram illustrating a conversational interface for providing selection information for a statistical algorithm selected with respect to a factor analysis for categorical response prediction (Part 4-4).

FIG. 57 is a flowchart illustrating a whole process for selecting a statistical algorithm according to variable characteristics (Part 4-4) in variable characteristic information selected with respect to the purpose of use of a statistical analysis program.

FIGS. 58 to 61 are diagrams illustrating a stage of setting, by a user, variables and parameters for each statistical algorithm through an interface.

FIG. 58 illustrates a stage of setting variables and parameters in the cases of ROC curve analysis (cutoff number=1) and cutoff analysis (cutoff number>1).

FIG. 59 illustrates a stage of setting variables and parameters in the cases of univariable binary logistic regression, univariable multinomial logistic regression, and univariable ordinal logistic regression.

FIG. 60 illustrates a stage of setting variables and parameters in the cases of multivariable binary logistic regression, multivariable multinomial logistic regression, and multivariable ordinal logistic regression.

FIG. 61 illustrates a stage of setting variables and parameters in the case of logistic mixed effect model analysis.

FIGS. 62 to 70 are diagrams illustrating a process of a case in which an example (Part 4-5; a survival data analysis) of a process of performing a clinical and statistical analysis method using a conversational interface according to the present disclosure is selected as answer information.

FIG. 62 is a diagram illustrating a conversational interface for selecting variable characteristics that are a combination of multiple conditions with respect to a survival data analysis (Part 4-5).

FIG. 63 is a diagram illustrating a conversational interface for providing selection information for a statistical algorithm selected with respect to a survival data analysis (Part 4-5).

FIG. 64 is a flowchart illustrating a whole process for selecting a statistical algorithm according to variable characteristics (Part 4-5) in variable characteristic information selected with respect to the purpose of use of a statistical analysis program.

FIGS. 65 to 70 are diagrams illustrating a stage of setting, by a user, variables and parameters for each statistical algorithm through an interface.

FIG. 65 illustrates a stage of setting variables and parameters in the case of time-dependent ROC curve analysis.

FIG. 66 illustrates a stage of setting variables and parameters in the case of Kaplan-Meier curve analysis.

FIG. 67 illustrates a stage of setting variables and parameters in the cases of univariable Cox proportional hazards regression analysis, and Cox regression using covariates with time-varying effect: univariable analysis.

FIG. 68 illustrates a stage of setting variables and parameters in the cases of multivariable Cox proportional hazards regression analysis, and Cox regression using covariates with time-varying effect: multivariable analysis.

FIG. 69 illustrates a stage of setting variables and parameters in the case of Cox regression using repeatedly measured covariates.

FIGS. 70 to 78 are diagrams illustrating a process of a case in which an example (Part 4-6; other analyses) of a process of performing a clinical and statistical analysis method using a conversational interface according to the present disclosure is selected as answer information.

FIG. 70 is a diagram illustrating a conversational interface for selecting variable characteristics that are a combination of multiple conditions with respect to other analyses (Part 4-6).

FIG. 71 is a diagram illustrating a conversational interface for providing selection information for a statistical algorithm selected with respect to other analyses (Part 4-6).

FIG. 72 is a flowchart illustrating a whole process for selecting a statistical algorithm according to variable characteristics (Part 4-6) in variable characteristic information selected with respect to the purpose of use of a statistical analysis program.

FIGS. 73 to 78 are diagrams illustrating a stage of setting, by a user, variables and parameters for each statistical algorithm through an interface.

FIG. 73 illustrates a stage of setting variables and parameters in the case of correlation analysis.

FIG. 74 illustrates a stage of setting variables and parameters in the case of linear mixed effect analysis.

FIG. 75 illustrates a stage of setting variables and parameters in the case of logistic mixed effect analysis.

FIG. 76 illustrates a stage of setting variables and parameters in the case of Cox mixed effect analysis.

FIG. 77 illustrates a stage of setting variables and parameters in the cases of Model Comparison I, and Model Comparison II.

FIG. 78 illustrates a stage of setting variables and parameters in the cases of Internal Validation I, and Internal Validation II.

FIG. 79 illustrates a stage of setting variables and parameters in the cases of External Validation I, and External Validation II.

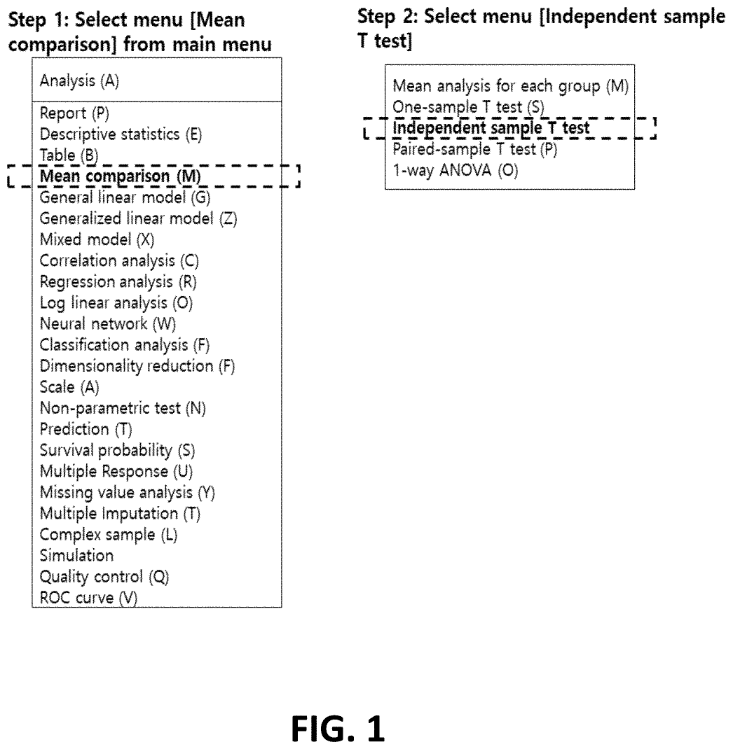

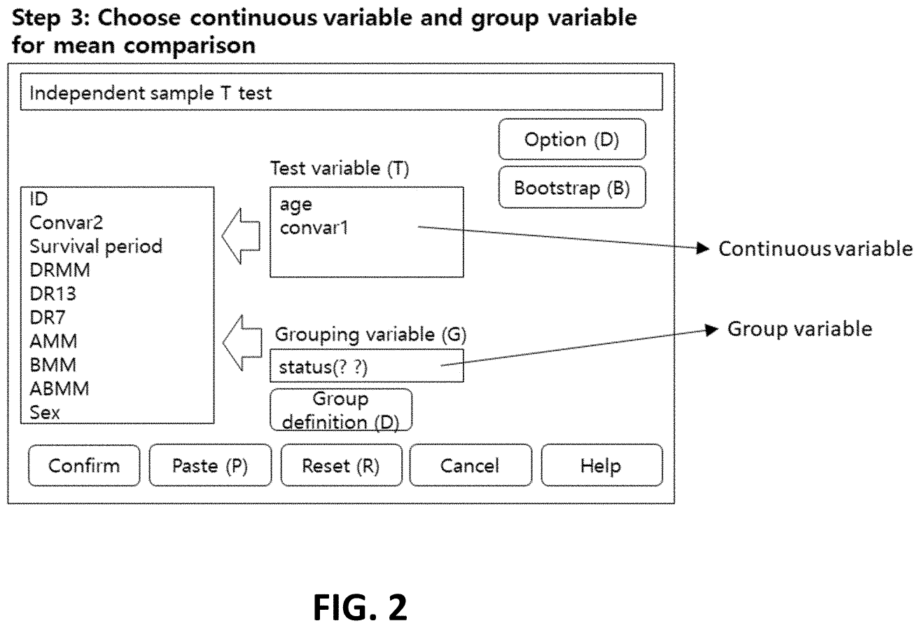

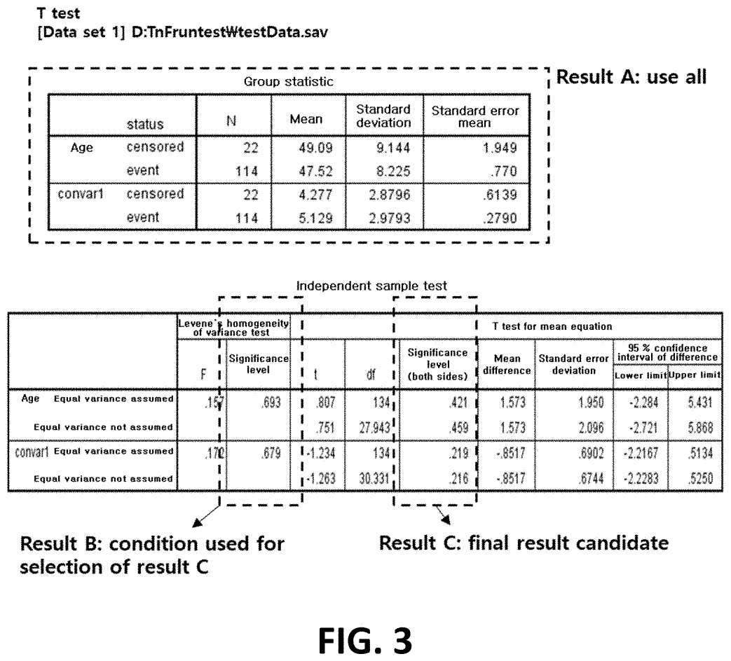

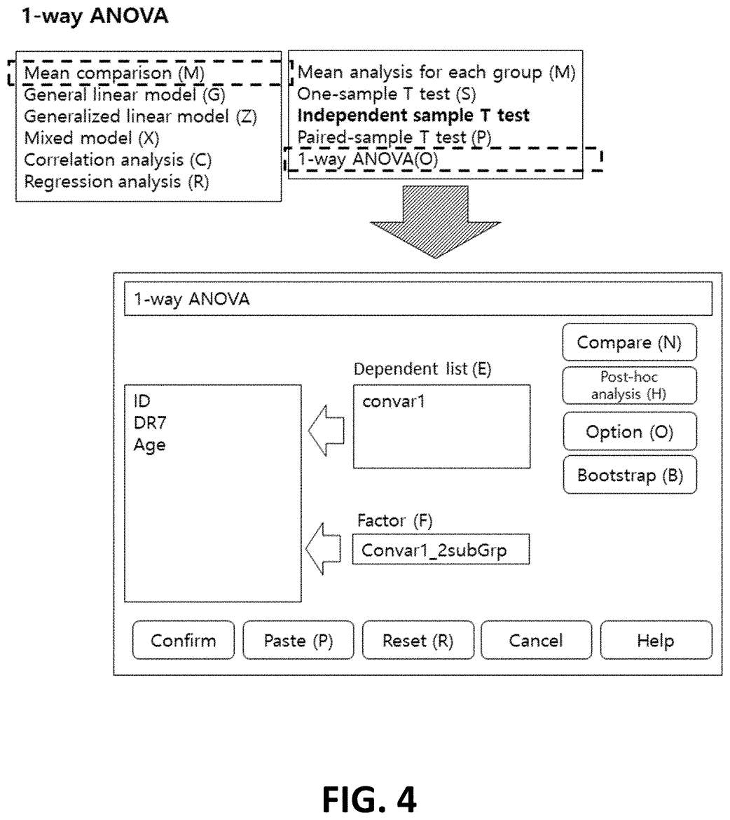

### BEST MODE

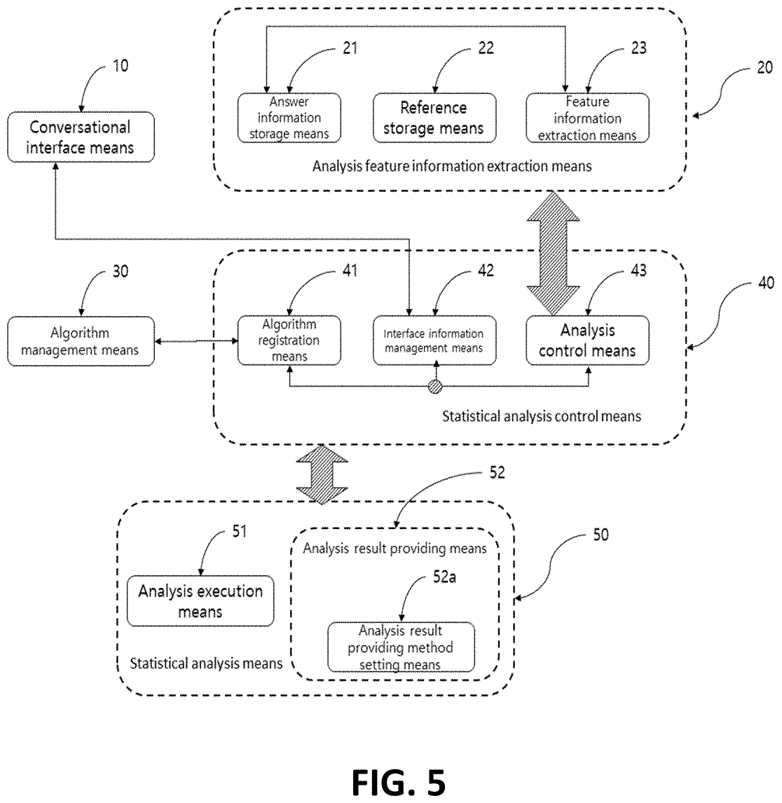

A statistical analysis system using a conversational interface according to the present disclosure will be described with reference to FIG. 5 as follows.

FIG. 5 is a block diagram illustrating a configuration of a statistical analysis system using a conversational interface according to the present disclosure.

The following are included: a conversational interface means 10 for providing inquiry information to a user to extract a user's purpose of a statistical analysis and variable information, and for collecting answer information provided from the user to an inquiry; an analysis feature information extraction means 20 for extracting analysis feature information that includes the user's purpose of the statistical analysis and the variable information, by using the answer information of the user acquired by the conversational interface means 10; an algorithm management means 30 for storing and managing statistical algorithms therein and for providing the statistical algorithms according to a request from a statistical analysis control means 40; the statistical analysis control means 40 for controlling execution of the statistical analysis in such a manner that the conversational interface is provided to the user through the conversational interface means 10, the analysis feature information is extracted through the analysis feature information extraction means 20 from the answer information collected through the conversational interface, and the statistical algorithm to be used in the statistical analysis is selected according to the analysis feature information; and a statistical analysis means 50 for performing the statistical analysis according to the statistical algorithm set by the statistical analysis control means 40 and for providing information about a result thereof.

According to the present disclosure, the statistical analysis system using the conversational interface has technical features that a user's intention is extracted through questions and answers with the user and analysis feature information which is a factor of the statistical analysis is extracted to enable a statistical algorithm suitable to the user's intention to be automatically applied.

The conversational interface means 10 is a means for providing an interface means for providing inquiry information to the user under the control of the statistical analysis control means 40 and enabling the user to input answer information.

The analysis feature information extraction means 20 is a means for extracting analysis feature information according to the user answer information input through the conversational interface means 10 under the control of the statistical analysis control means 40. The analysis feature information extraction means 20 includes: an answer information storage means 21 for collecting answer information from the conversational interface means 10 and managing the same; a reference storage means 22 for storing and managing reference information therein for extraction of the analysis feature information for each piece of the answer information for the inquiry information; and a feature information extraction means 23 for extracting analysis feature information from the stored answer information by referring to the reference information of the reference storage means 22.

The algorithm management means 30 is a means for storing and managing statistical algorithms therein and for providing the statistical algorithms according to a request from the statistical analysis control means 40.

Each of the statistical algorithms is a process of how to perform a statistical analysis, and in this embodiment, the following statistical algorithms are provided.

Two-sample T test, paired T test, 1-way ANOVA, repeated measures 1-way ANOVA, repeated measures 2-way ANOVA, 1-way ANCOVA, 1-way ANCOVA with repeated measures, and 2-way ANCOVA with repeated measures.

The statistical algorithms are not limited to the above examples, and more various statistical algorithms may be registered and stored.

The statistical analysis control means 40 is a control means for performing a statistical analysis by making an inquiry to the user through the conversational interface, by making analysis feature information extracted according to answer information, and by setting a statistical algorithm accordingly.

The statistical analysis control means 40 includes: an algorithm registration means 41 for enabling a program producer or system manager to additionally register and store a statistical algorithm in the algorithm management means 30 or to delete the previously registered statistical algorithm when necessary; an interface information management means 42 for storing and managing interface information therein for making conversation with the user to extract the purpose of use of a statistical analysis program and variable feature information for each purpose of use of the statistical analysis program and for receiving answer information from the user; and an analysis control means 43 for providing the analysis feature information extraction means 20 with control information for extracting analysis feature information from the answer information of the user so that the analysis feature information is extracted by the analysis feature information extraction means 20, and for providing control information to the statistical analysis means 50 so that the algorithm of the algorithm management means 30 is set from the analysis feature information extracted by the analysis feature information extraction means 20 and the statistical analysis is performed.

The statistical analysis means 50 is a means for executing a statistical analysis according to the control information of the analysis control means 43. The statistical analysis means 43 includes: an analysis execution means 51 for executing a statistical analysis according to a process provided by the statistical algorithm selected by the statistical analysis control means 40; and an analysis result providing means 52 for providing analysis result information acquired through the analysis execution means 51.

The analysis result providing means 52 may further include an analysis result providing method setting means 52a for enabling the user to set a method of providing the analysis result information.

The operation of the statistical analysis system using the conversational interface according to the present disclosure having such a configuration will be described as follows.

In the present disclosure, a conversational interface is provided to the user in such a manner that an inquiry is provided to the user, an answer thereto is received, and from association information, the user's purpose of performing a statistical analysis, the characteristics of variables to be applied, etc. are identified.

The statistical analysis control means 40 provides inquiry information through the conversational interface means 10, and the user inputs answer information to the inquiry information through the conversational interface means 10.

The statistical analysis control means 40 stores the answer information in the answer information storage means 21 of the analysis feature information extraction means 20, and selects inquiry information for the next step according to the answer information to provide the inquiry information to the conversational interface means 10.

Repeating the above stage, each time the user inputs answer information the answer information is stored in the answer information storage means 21 of the analysis feature information extraction means 20. When providing of inquiry information according to conversational interface information provided by the statistical analysis control means 40 is completed, the analysis control means 43 of the statistical analysis control means 40 provides control information to the analysis feature information extraction means 20 and analysis feature information is extracted using the answer information stored in the answer information storage means 21 and input by the user.

The feature information extraction means 23 of the analysis feature information extraction means 20 extracts feature information by loading reference information for extracting analysis feature information from the reference storage means 22 by using the answer information of the user stored in the answer information storage means 21.

The analysis control means 43 classifies the answer information according to the reference information and generates analysis purpose information of the user, types of variables to be applied, and association information between the variables so that the analysis feature information is extracted.

After extraction of analysis feature information is completed by the feature information extraction means 23, the analysis control means 43 sets the statistical analysis algorithm matched to the extracted analysis feature information among the statistical analysis algorithms registered in the algorithm management means 30 as an algorithm to be applied to a statistical analysis of the statistical analysis means 50.

Afterward, the analysis execution means 51 of the statistical analysis means 50 performs the statistical analysis according to a process of the statistical algorithm set as described above, and the analysis result providing means 52 provides result information according to a method that the user selects.

As described above, the user is sequentially provided with inquiries related to the purpose of an analysis and variables of types required for the analysis, and is continuously provided with inquiries determined according to answer information, so that the user's purpose of an analysis and pieces of variable information required for the analysis are acquired.

In the meantime, a process of performing, by the statistical analysis control means 40, a clinical and statistical analysis method using the conversational interface will be described as follows.

The following are included: a feature information extraction induction stage in which when the statistical analysis system starts, the conversational interface means 10 for conversation with a user is controlled to provide the conversational interface and through the conversational interface, inquiries are made to the user, and answer information to the inquiries is acquired and stored; an analysis feature information extraction stage in which after the feature information extraction induction stage is completed, the analysis feature information extraction means 20 is controlled to extract, from the answer information, the purpose of a statistical analysis that the user wants and variable characteristic information to be applied to the statistical analysis; an algorithm setting stage in which an algorithm matched to the purpose of the statistical analysis and to the variable characteristic information extracted by the analysis feature information extraction stage 20 is selected from the algorithm management means 30 and is set in the statistical analysis means 50; a statistical analysis execution stage in which the statistical analysis means 50 is controlled to execute the statistical analysis according to the set algorithm; and a statistical analysis result providing stage in which results acquired through the statistical analysis execution stage are provided to the user through the conversational interface means 10.

The feature information extraction induction stage includes: a purpose inquiry stage in which when the statistical analysis system starts, a request to start a conversation analysis is made, and when a request to start the conversation analysis is made from the user, the user is provided with an inquiry for acquiring the purpose of use of the program and an answer thereto is input; and a variable characteristic information inquiry stage in which when the answer is input through the purpose inquiry stage, the answer is stored in the analysis feature information extraction means, inquiry information is provided to create a variable characteristic table corresponding to the user's selected purpose of the statistical analysis, and answer information is input and stored in the analysis feature information extraction means.

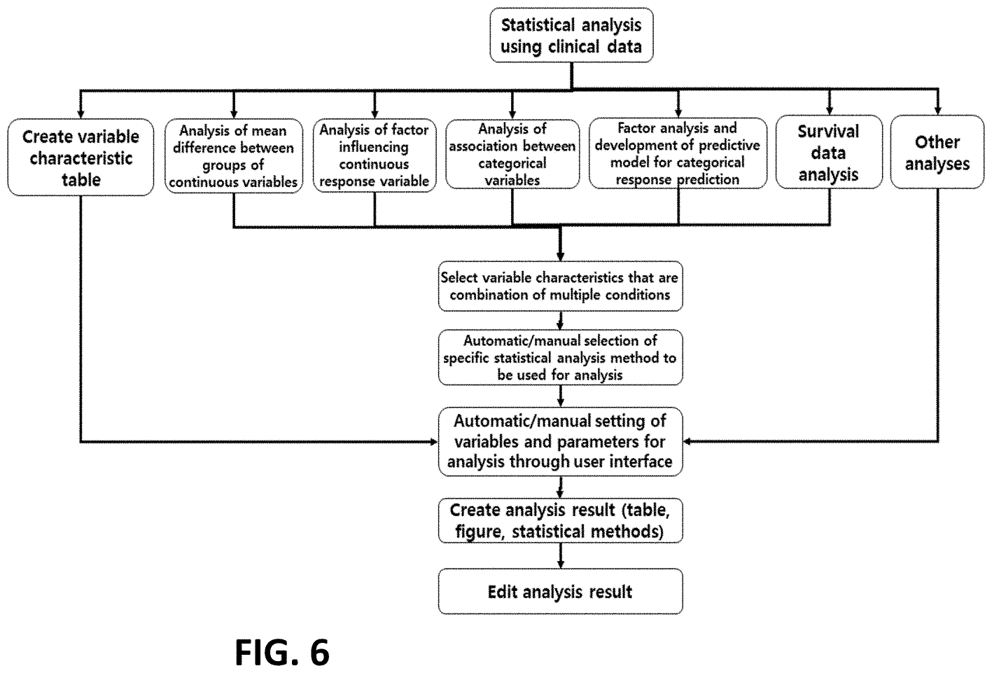

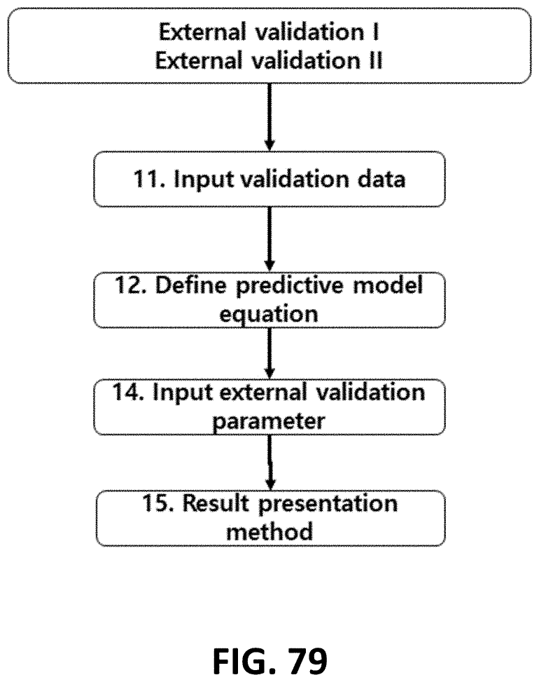

FIGS. 6 to 79 illustrates an example of a process of performing a clinical and statistical analysis method using the conversational interface according to the present disclosure, and referring to this, a detailed description will be provided as follows.

FIG. 6 is a flowchart illustrating a whole process of a statistical analysis method using clinical data.

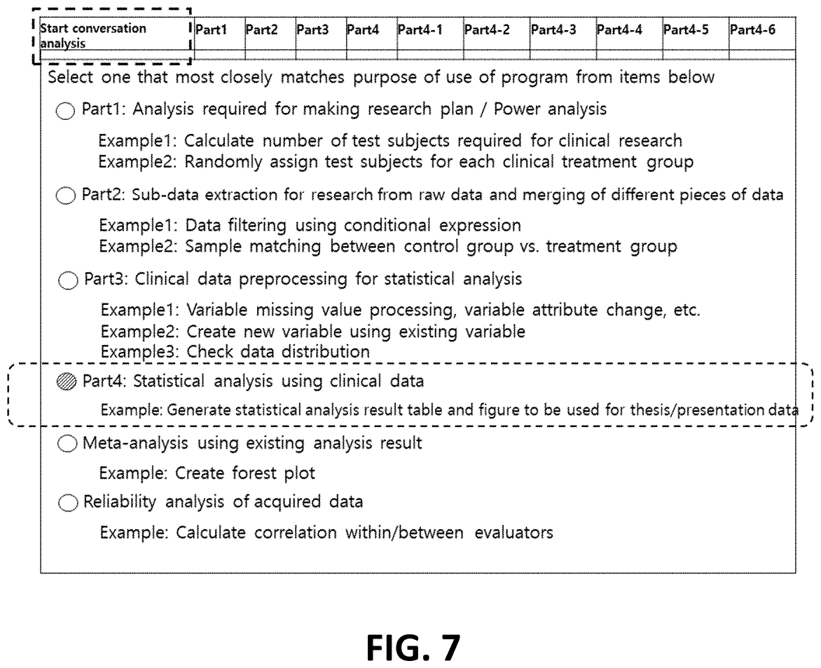

FIG. 7 illustrates an example of a conversational interface including inquiry information provided to a user to acquire the purpose of a statistical analysis.

When a statistical analysis process starts, the purpose inquiry stage for acquiring the purpose of a statistical analysis is performed, and the conversational interface for inquiring the purpose of use of the program is provided as shown in FIG. 7.

In the purpose inquiry stage, an analysis required for making a research plan, sub-data extraction for a research from raw data and merging of different pieces of data, clinical data preprocessing for a statistical analysis, a statistical analysis using clinical data, a meta-analysis using an existing analysis result, and a reliability analysis of acquired data may be included.

As shown in FIG. 7, at the upper part of the conversational interface, bookmarks for respective inquiry stages (respective Parts) to proceed according to a process are provided, and at the lower part, inquiry information for each step is provided, and at a part of the right side, a link mark for proceeding from the current step to the next step is provided.

Under the answer information that may be selected for the inquiry information in each purpose inquiry stage, example information is provided so that the user can easily select the answer information, that is, an answer to the inquiry information.

In this embodiment, it is shown that the user recognizes “Example: generate a statistical analysis result table and a figure to be used for thesis/presentation data” and selects “Part 4: a statistical analysis using clinical data”.

When a statistical analysis (Part 4) using clinical data is selected as described above, Part 4 is executed to create a variable characteristic table for acquiring variable characteristic information, and FIG. 7 illustrates inquiry information for creating the variable characteristic table.

The inquiry information for creating the variable characteristic table may include an analysis of a mean difference between groups of continuous variables (Part 4-1), an analysis of a factor influencing a continuous response variable (Part 4-2), an analysis of association between categorical variables (Part 4-3), a factor analysis and development of a predictive model for categorical response prediction (Part 4-4), a survival data analysis (Part 4-5), and other analyses (Part 4-6).

Each of the Parts for inputting the answer information to each piece of the inquiry information includes example information.

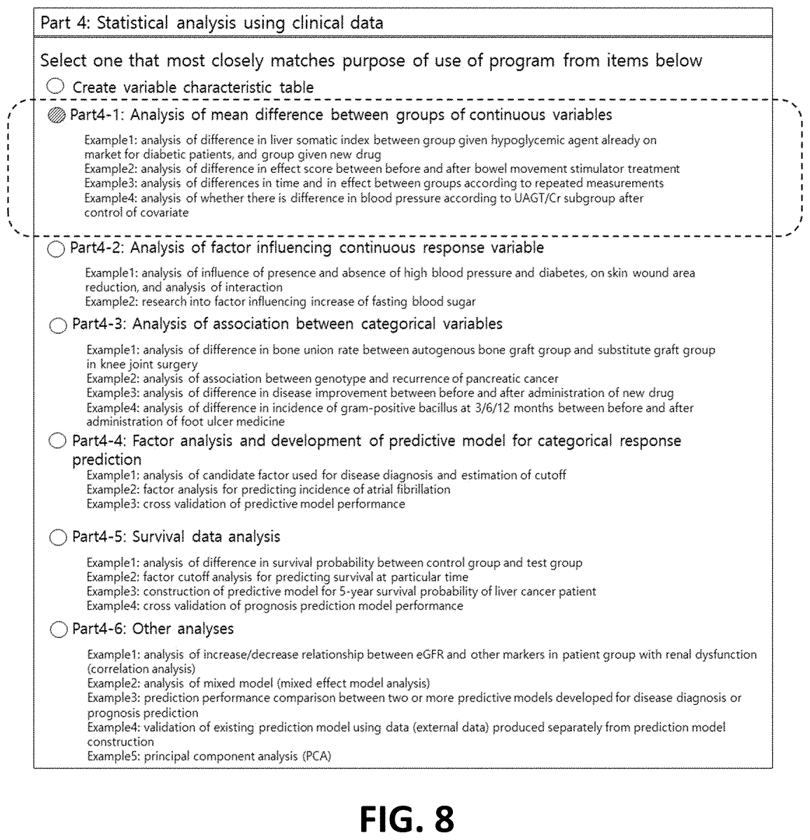

FIG. 8 illustrates inquiry information for a statistical analysis for clinical data selected in the purpose inquiry stage and the user selects an analysis of a mean difference between groups of continuous variables (Part 4-1) as answer information.

In the conversational interface for creating the variable characteristic table as shown in FIG. 8, at a part of the right side thereof, a link button is provided for proceeding to the next step or the previous step.

As shown in FIG. 7, at the first step, a link mark for only proceeding to the next step is provided, but as shown in FIG. 8 and so forth, when there is the previous step, link marks for proceeding to the next step or back to the previous step are provided.

As described above, a statistical algorithm is selected by extracting information on variable characteristics with respect to PART that the user selects among pieces of selection information as shown in FIG. 7 for creating a variable characteristic table.

In an embodiment of the present disclosure, “an analysis of a mean difference between groups of continuous variables” (Part 4-1) will be described as follows.

Part 4-1; a process for “an analysis of a mean difference between groups of continuous variables”.

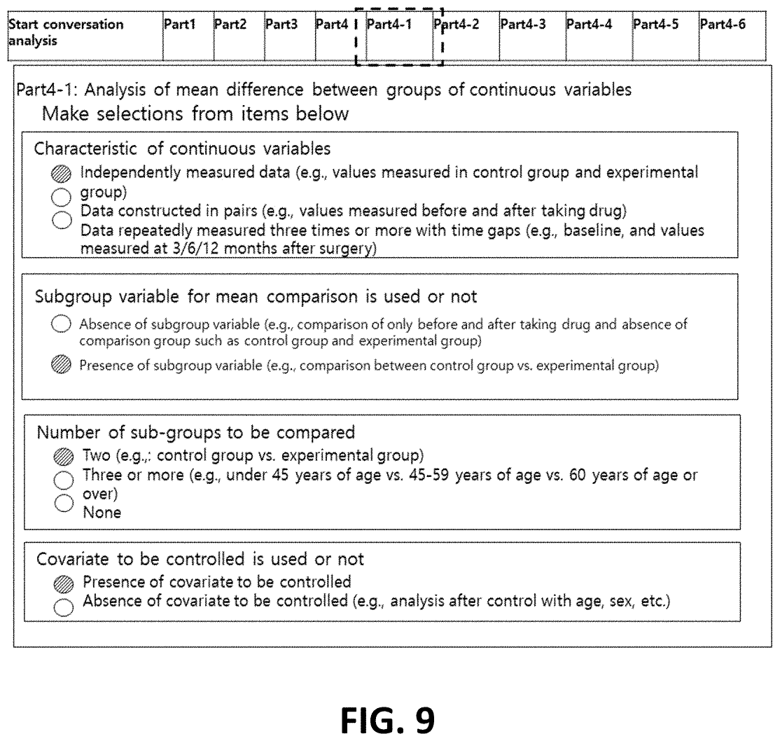

As shown in FIG. 8, when an analysis of a mean difference between groups of continuous variables (Part 4-1) is selected, a process for selecting variable characteristics that are a combination of multiple conditions of FIG. 9 with respect to an analysis of a mean difference between groups of continuous variables proceeds.

The analysis of a mean difference between groups of continuous variables includes inquiry information on the characteristic of continuous variables, whether a subgroup variable for mean comparison is used, the number of subgroups to be compared, and whether a covariate to be controlled is present.

Regarding to the characteristic of continuous variable, inquiry information for identifying whether data is independently measured, whether data is constructed in pairs, and whether data is repeatedly measured at least three times with time gaps is included. Regarding the number of subgroups to be compared, inquiry information is generated depending on whether a subgroup variable for mean comparison is used.

Through this process, when the user's purpose of use of the program and the variable characteristic information are acquired, a statistical algorithm is selected accordingly.

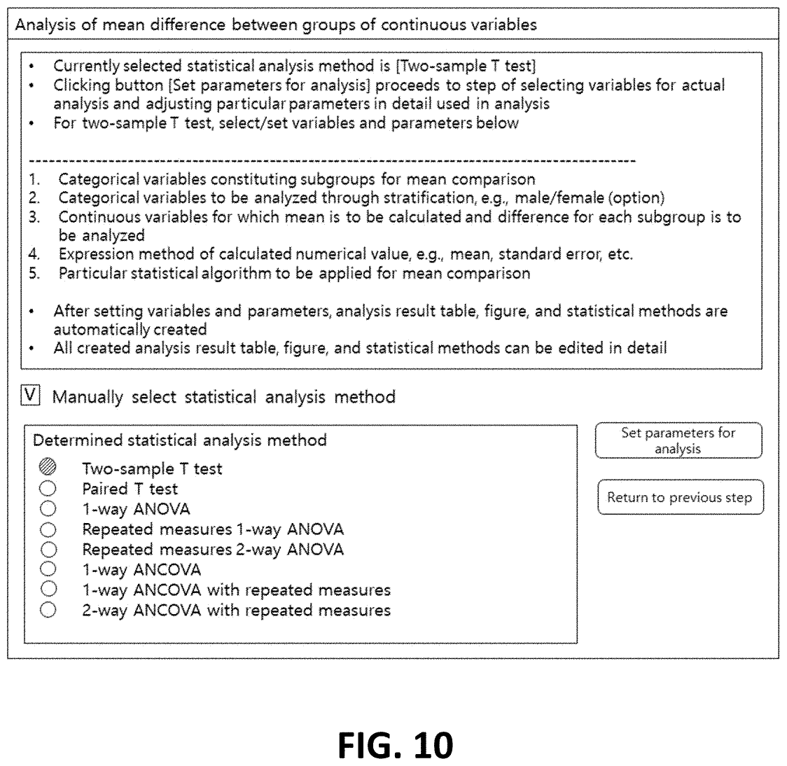

FIG. 10 illustrates a conversational interface for providing selection information for a statistical algorithm selected with respect to the analysis of a mean difference between groups of continuous variables as described above.

The selection information includes variable and parameter setting information for making a statistical analysis execution stage proceed for the selected statistical algorithm, and further includes item information that enables the user to manually select a statistical analysis.

As shown in FIG. 10, a conversational interface is to provide selection information for a statistical algorithm. At the upper part of the conversational interface for selecting a specific statistical algorithm to be used for an analysis, current program use purpose information (an analysis of a mean difference between groups of continuous variables) is displayed. In addition, inside the lower part, a statistical algorithm currently analyzed is displayed, and pieces of guide information to be selected/set for setting variables and parameters are included so that the statistical algorithm proceeds. A link button for setting variables and parameters, which is the next process according to the user's selection, is included.

In addition, an automatic/manual selection item for enabling the user to manually select a statistical algorithm is included, and statistical algorithm information that may be selected when manual selection is selected with respect to the selection item is further included.

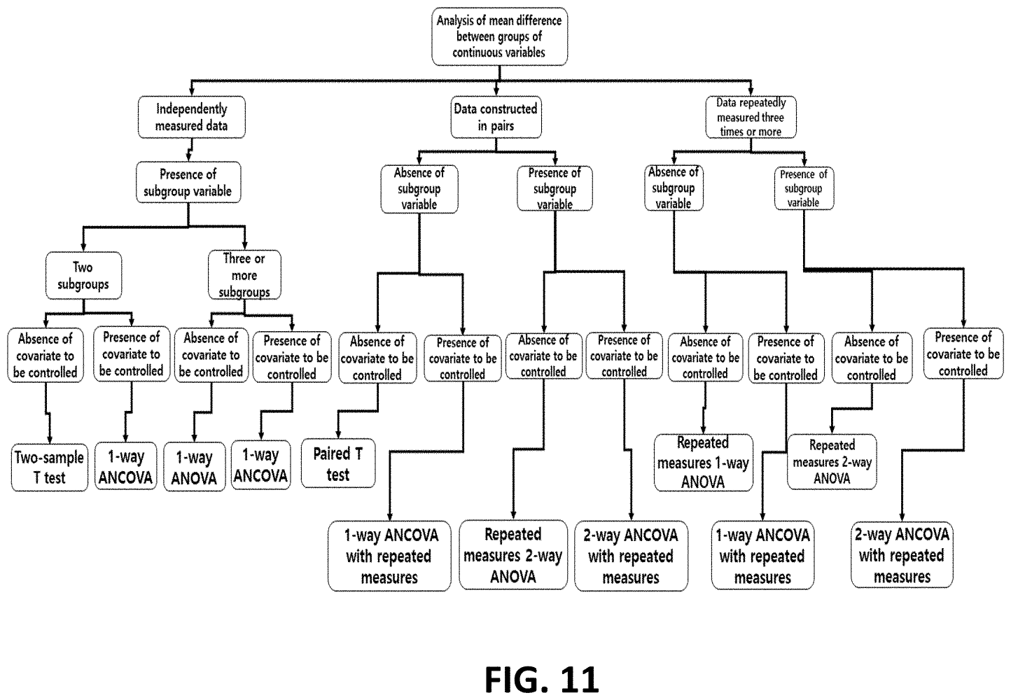

FIG. 11 is a flowchart illustrating a whole process for selecting a statistical algorithm according to variable characteristics in the variable characteristic information selected with respect to the purpose of use of the statistical analysis program.

In the analysis of a mean difference between groups of continuous variables, the process of selecting a statistical algorithm is composed of: a stage of determining the characteristic of continuous variables to identify whether data is independently measured, whether data is constructed in pairs, or whether data is repeatedly measured at least three times with time gaps; a stage of determining whether a subgroup variable is used and determining the number of subgroups to be compared; a stage of determining whether a covariate to be controlled is used; and a stage of selecting a statistical algorithm according to a result determined through the above stage.

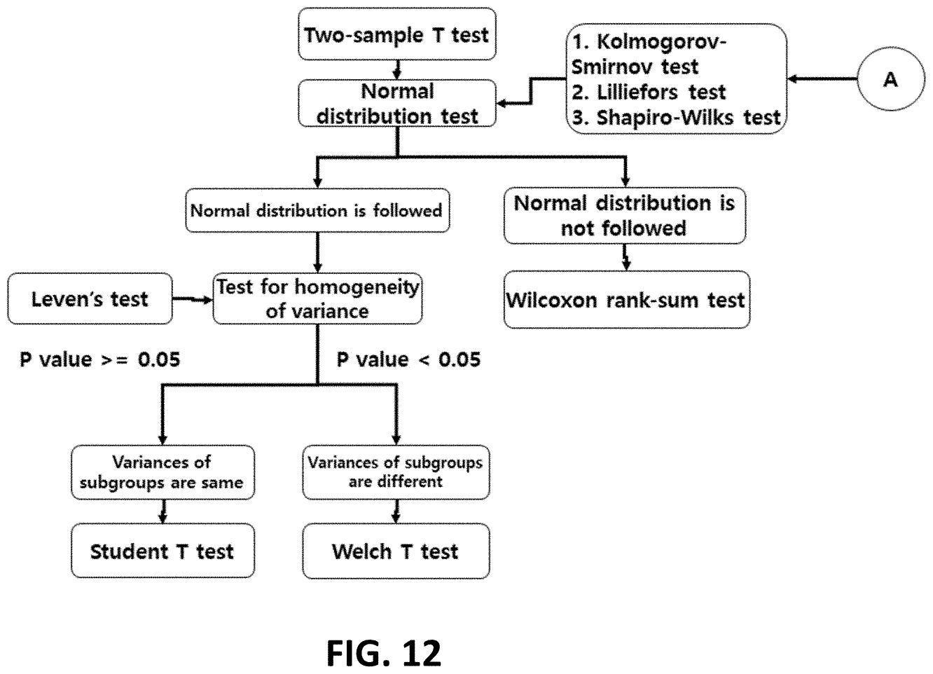

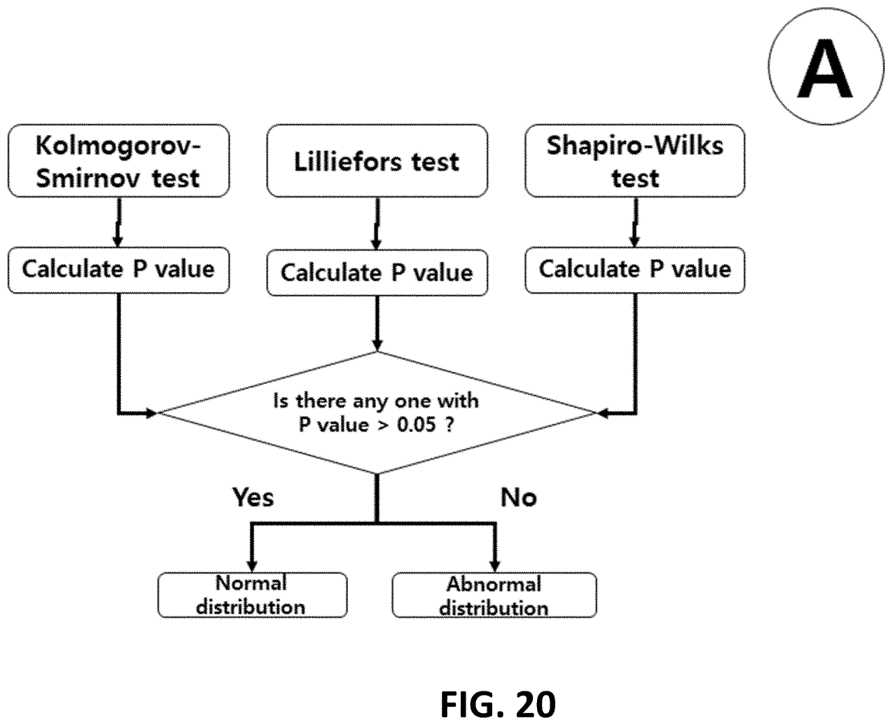

FIGS. 12 to 19 are flowchart illustrating a method of automatically selecting a particular algorithm, in each algorithm. FIG. 20 is a flowchart illustrating a normal distribution test method A for variable data, in the method of automatically selecting a particular algorithm of FIGS. 12 to 19.

First, FIG. 12 is a flowchart illustrating a stage of automatically selecting a particular algorithm of algorithm “two-sample T test”.

The following are included: a stage of performing a normal distribution test first and determining a case in which normal distribution is followed or a case in which normal distribution is not followed; a stage of selecting a statistical algorithm corresponding to the case in which normal distribution is not followed; a stage of performing a test (Levene's test) for homogeneity of variance in the case in which normal distribution is followed; and a stage of comparing a significance probability value (P-Value) that is a result of the test for homogeneity of variance with a reference value and determining an algorithm by determining a case in which variances of the subgroups are the same or a case in which variances of the subgroups are different.

A stage of setting the reference value of the significance probability value (P-Value) by the user may be further included.

As the significance probability value (P-Value), 0.05 is generally applied, but the user may select and set other values.

FIG. 20 is a flowchart illustrating the normal distribution test process A for the variable data.

The following are included: a stage of calculating a significance probability value (P-Value) through one or more normal distribution test algorithms (Kolmogorov-Smirnov test, Lilliefors test, and Shapiro-Wiks test); and a stage of comparing the P-Value acquired through the above stage with a reference value (0.05) and determining a case in which normal distribution is followed (normal distribution) or a case in which normal distribution is not followed (abnormal distribution).

Regarding the criterion for determining the normal distribution/abnormal distribution, it is determined whether there is a P-Value greater than the reference value, and when there is even one, normal distribution is determined.

According to the result of the normal distribution test, when normal distribution is not followed, selection is made using a statistical algorithm (Wilcoxon rank sum test).

When normal distribution is followed, a test (Leven's test) for homogeneity of variance is performed. When the P-Value is equal to or greater than the reference value (0.05), it is determined that variances of the subgroups are the same. When the P-Value is less than the reference value (0.05), it is determined that variances of the subgroups are different.

As the result of the test for homogeneity of variance, when variances of the subgroups are the same, statistical algorithm “Student T test” is selected. When variances of the subgroups are different, statistical algorithm “Welch T test” is selected.

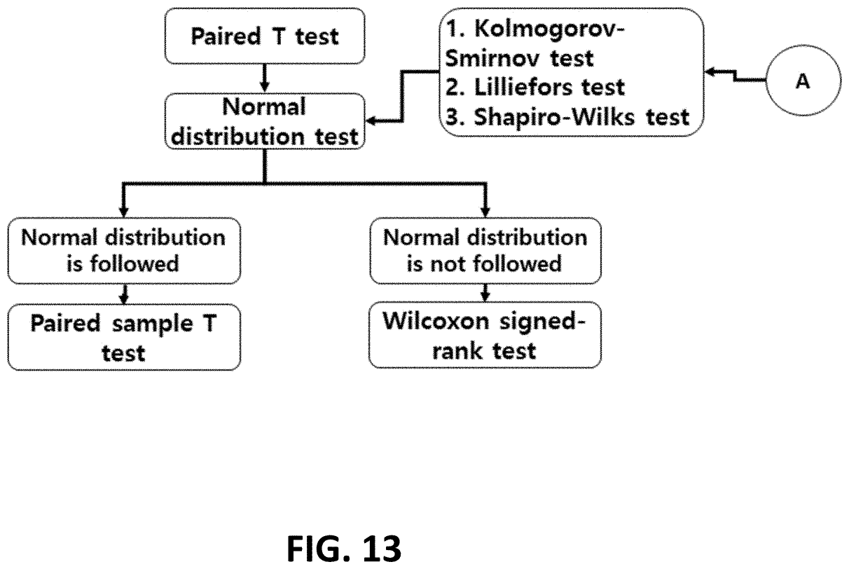

FIG. 13 is a flowchart illustrating a stage of automatically selecting a particular algorithm of algorithm “paired T test”.

In “paired T test”, a normal distribution test as shown in FIG. 20 is performed. When normal distribution is followed, “paired sample t test” is selected. When normal distribution is not followed, “Wilcoxon signed rank test” is selected.

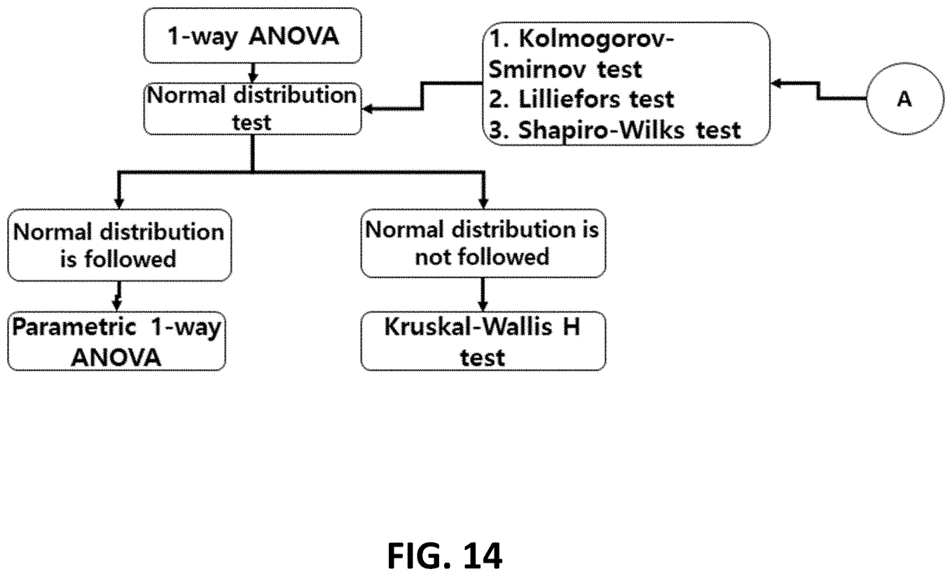

FIG. 14 is a flowchart illustrating a stage of automatically selecting a particular algorithm of algorithm “1-way ANOVA”.

In “1-way ANOVA”, a normal distribution test as shown in FIG. 20 is performed. When normal distribution is followed, “parametric 1-way ANOVA” is selected. When normal distribution is not followed, “Kruskal-Wallis H test” is selected.

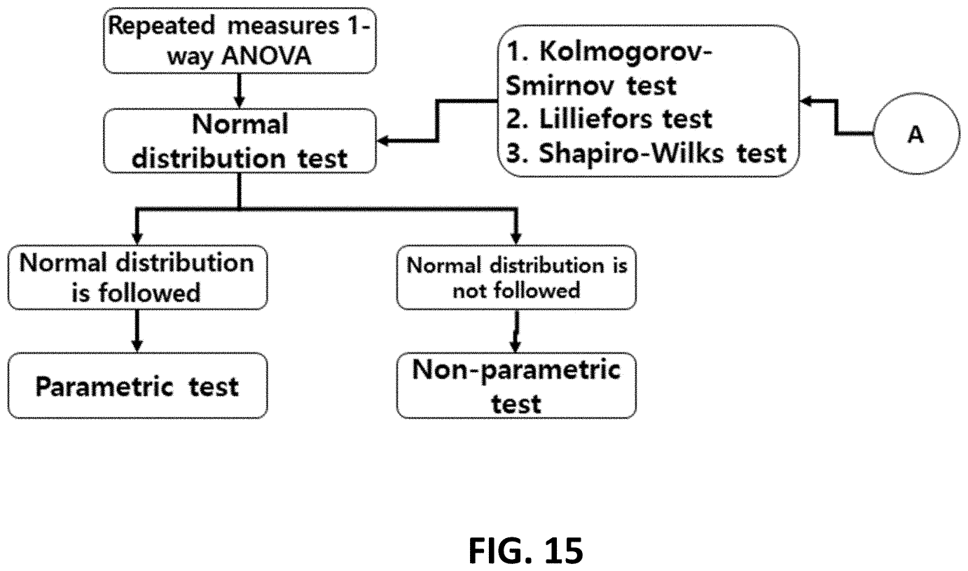

FIG. 15 is a flowchart illustrating a stage of automatically selecting a particular algorithm of algorithm “repeated measures 1-way ANOVA”.

In “repeated measures 1-way ANOVA”, a normal distribution test as shown in FIG. 20 is performed. When normal distribution is followed, “parametric test” is selected. When normal distribution is not followed, “non-parametric test” is selected.

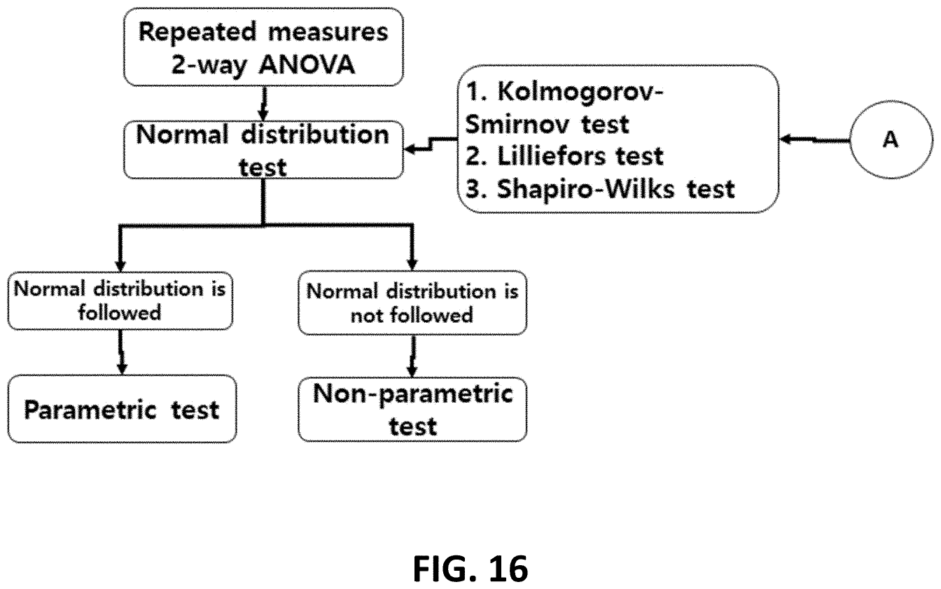

FIG. 16 is a flowchart illustrating a stage of automatically selecting a particular algorithm of algorithm “repeated measures 2-way ANOVA”.

In “repeated measures 2-way ANOVA”, a normal distribution test as shown in FIG. 20 is performed. When normal distribution is followed, “parametric test” is selected. When normal distribution is not followed, “non-parametric test” is selected.

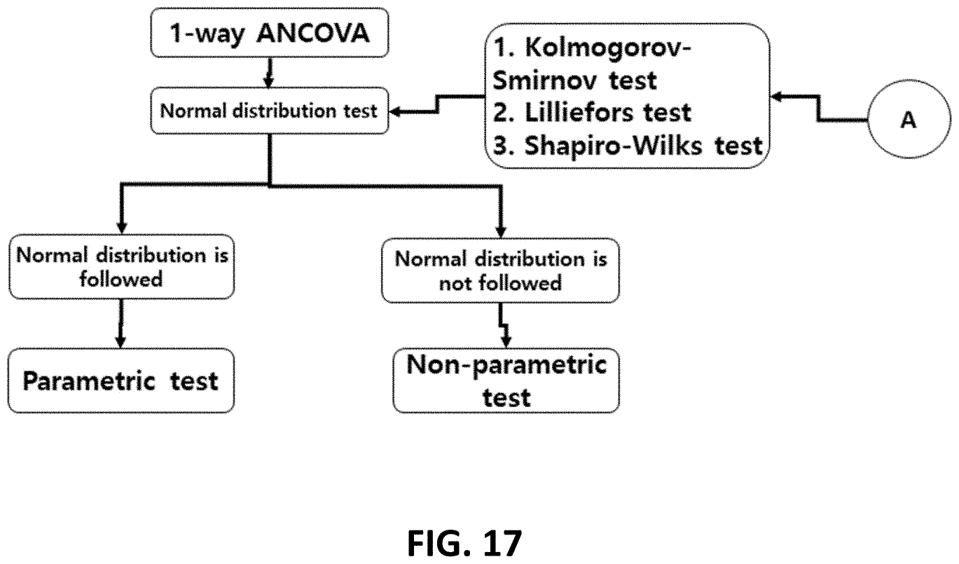

FIG. 17 is a flowchart illustrating a stage of automatically selecting a particular algorithm of algorithm “1-way ANCOVA”.

In “1-way ANCOVA”, a normal distribution test as shown in FIG. 20 is performed. When normal distribution is followed, “parametric test” is selected. When normal distribution is not followed, “non-parametric test” is selected.

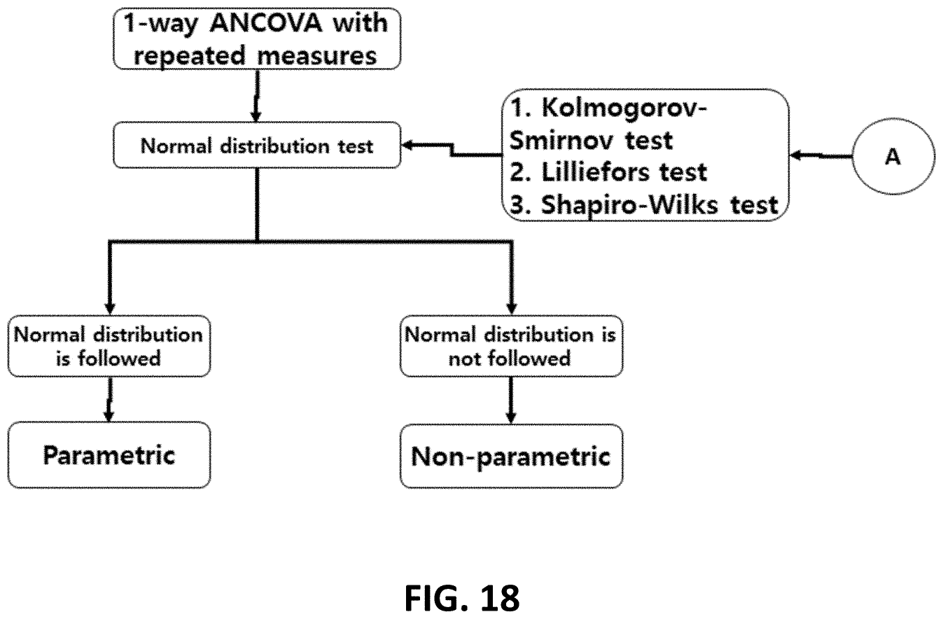

FIG. 18 is a flowchart illustrating a stage of automatically selecting a particular algorithm of algorithm “1-way ANCOVA with repeated measures”.

In “1-way ANCOVA with repeated measures”, a normal distribution test as shown in FIG. 20 is performed. When normal distribution is followed, “parametric test” is selected. When normal distribution is not followed, “non-parametric test” is selected.

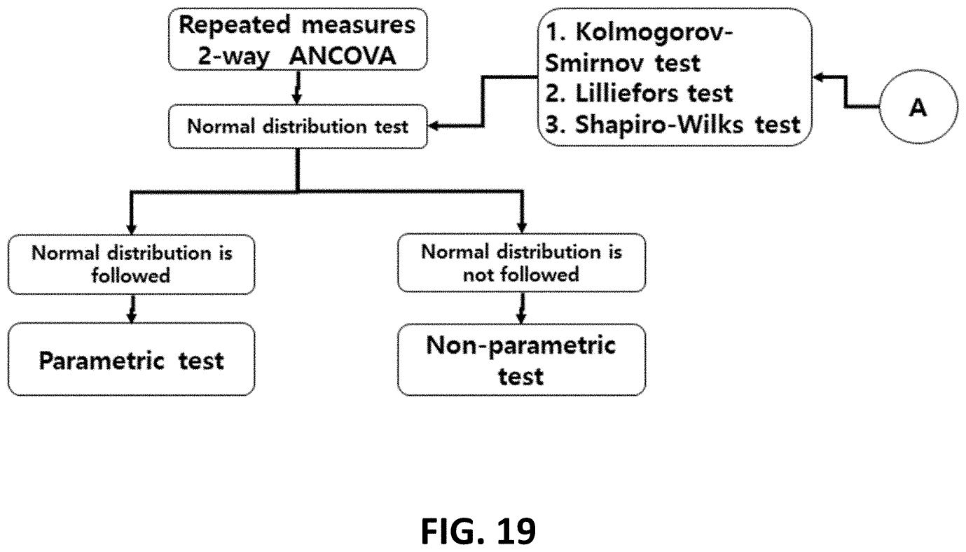

FIG. 19 is a flowchart illustrating a stage of automatically selecting a particular algorithm of algorithm “2-way ANCOVA with repeated measures”.

In “2-way ANCOVA with repeated measures”, a normal distribution test as shown in FIG. 20 is performed. When normal distribution is followed, “parametric test” is selected. When normal distribution is not followed, “non-parametric test” is selected.

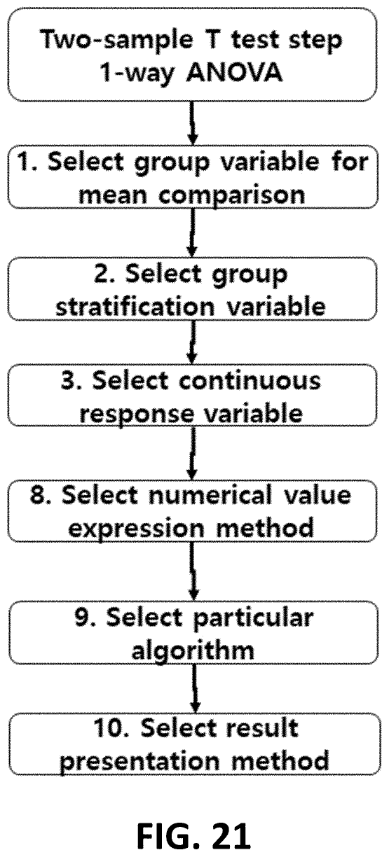

FIGS. 21 to 27 are diagrams illustrating a stage of setting, by the user, variables and parameters for each statistical algorithm through the interface.

FIG. 21 illustrates a stage of setting variables and parameters in the cases of “two-sample T test” and “1-way ANOVA”.

“Two-sample T test” and “1-way ANOVA” are composed of a stage of selecting a group variable for mean comparison, a stage (option) of selecting a group stratification variable according to user's selection, a stage of selecting a continuous response variable, a stage of selecting a numerical value expression method, a stage of selecting a particular algorithm, and a stage of selecting a result presentation method.

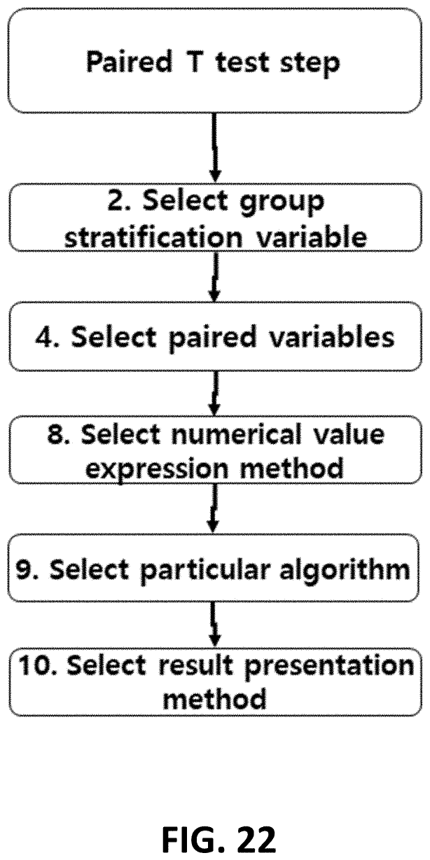

FIG. 22 illustrates a stage of setting variables and parameters in the case of “paired T test”.

“Paired T test” is composed of a stage (option) of selecting a group stratification variable, a stage of selecting paired variables, a stage of selecting a numerical value expression method, a stage of selecting a particular algorithm, and a stage of selecting a result presentation method.

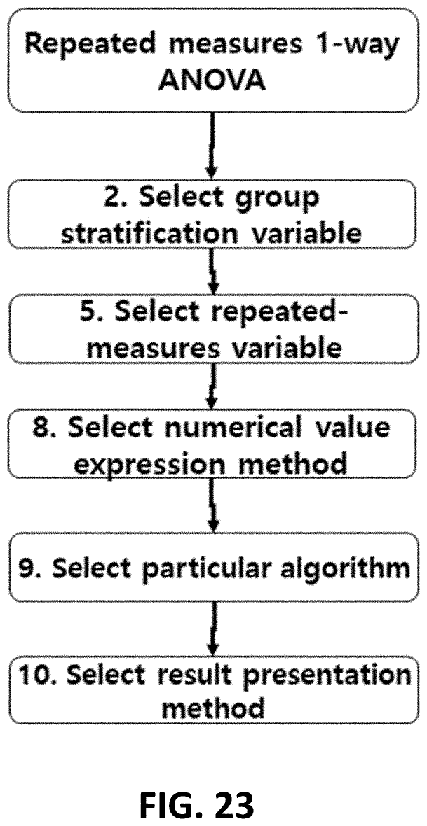

FIG. 23 illustrates a stage of setting variables and parameters in the case of “repeated measures 1-way ANOVA”.

“Repeated measures 1-way ANOVA” is composed of a stage (option) of selecting a group stratification variable, a stage of selecting a repeated-measures variable, a stage of selecting a numerical value expression method, a stage of selecting a particular algorithm, and a stage of selecting a result presentation method.

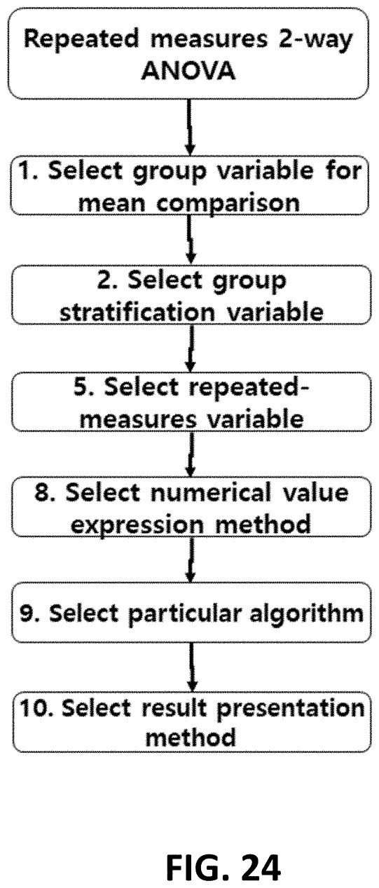

FIG. 24 illustrates a stage of setting variables and parameters in the case of “repeated measures 2-way ANOVA”.

“Repeated measures 2-way ANOVA” is composed of a stage of selecting a group variable for mean comparison, a stage (option) of selecting a group stratification variable, a stage of selecting a repeated-measures variable, a stage of selecting a numerical value expression method, a stage of selecting a particular algorithm, and a stage of selecting a result presentation method.

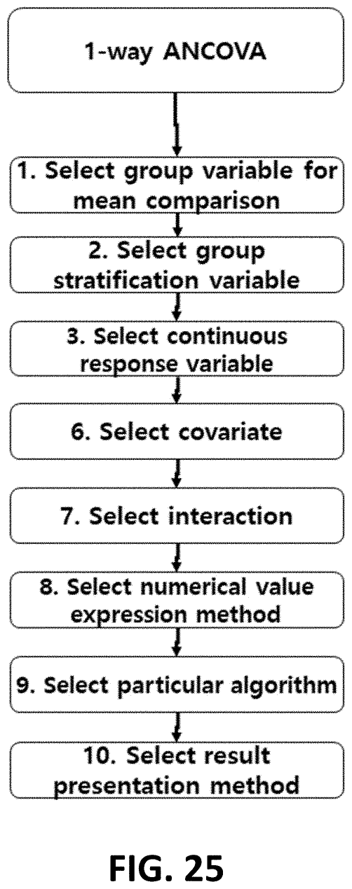

FIG. 25 illustrates a stage of setting variables and parameters in the case of “1-way ANCOVA”.

“1-way ANCOVA” is composed of a stage of selecting a group variable for mean comparison, a stage (option) of selecting a group stratification variable, a stage of selecting a continuous response variable, a stage of selecting a covariate, a stage (option) of selecting interaction, a stage of selecting a repeated-measures variable, a stage of selecting a numerical value expression method, a stage of selecting a particular algorithm, and a stage of selecting a result presentation method.

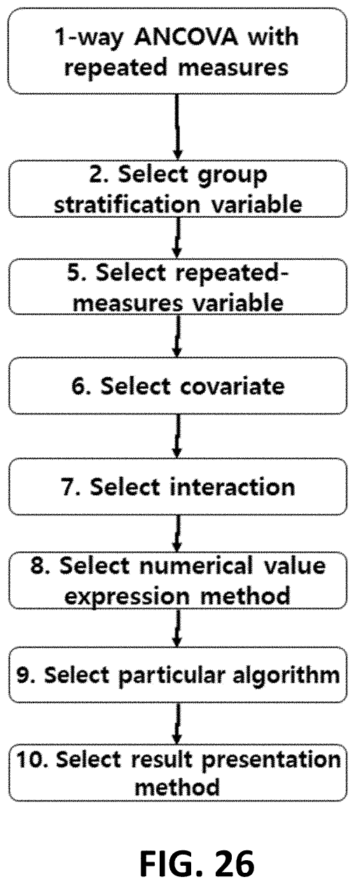

FIG. 26 illustrates a stage of setting variables and parameters in the case of “1-way ANCOVA with repeated measures”.

“1-way ANCOVA with repeated measures” is composed of a stage (option) of selecting a group stratification variable, a stage of selecting a repeated-measures variable, a stage of selecting a covariate, a stage (option) of selecting interaction, a stage of selecting a numerical value expression method, a stage of selecting a particular algorithm, and a stage of selecting a result presentation method.

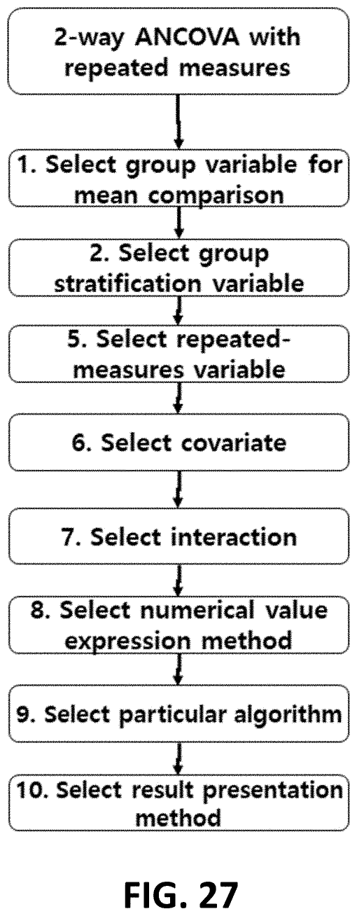

FIG. 27 illustrates a stage of setting variables and parameters in the case of “2-way ANCOVA with repeated measures”.

“2-way ANCOVA with repeated measures” is composed of a stage of selecting a group variable for mean comparison, a stage (option) of selecting a group stratification variable, a stage of selecting a repeated-measures variable, a stage of selecting a covariate, a stage (option) of selecting interaction, a stage of selecting a numerical value expression method, a stage of selecting a particular algorithm, and a stage of selecting a result presentation method.

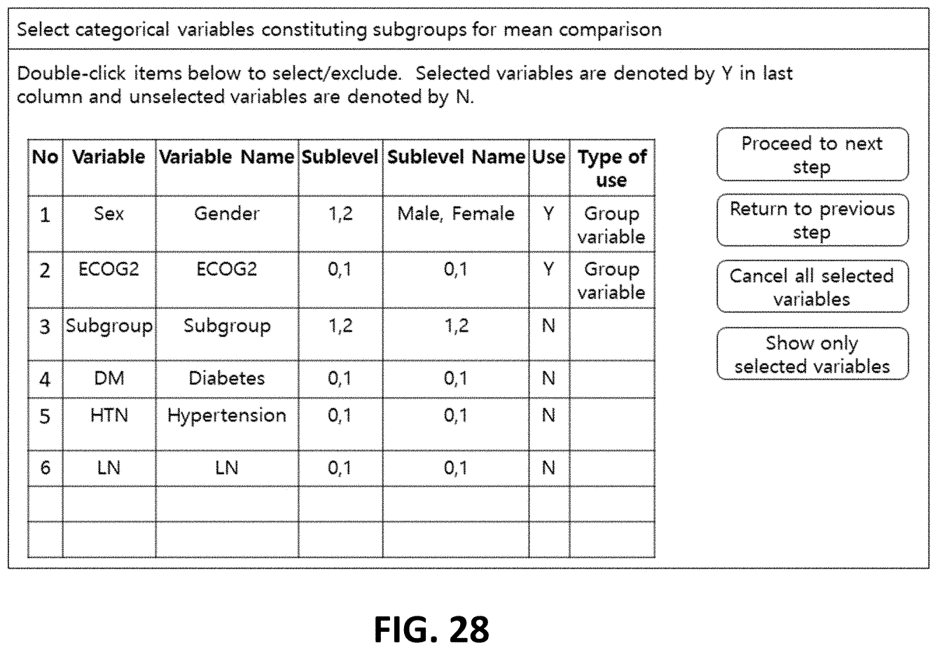

FIG. 28 illustrates an example of a user interface for performing a stage of selecting a group variable for mean comparison, in the stage of setting variables and parameters.

The user interface for selecting a group variable for mean comparison is configured in such a manner that variables are provided to select categorical variables constituting subgroups for mean comparison and the user is able to select (double-click) each of the variables.

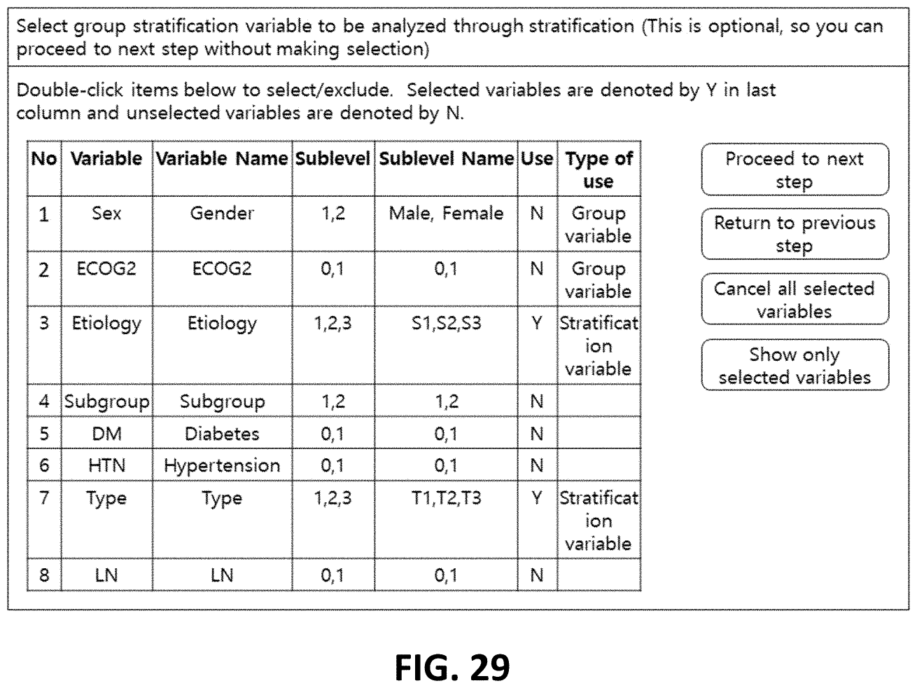

FIG. 29 illustrates an example of a user interface for performing selection of a group stratification variable, in the stage of setting variables and parameters.

The user interface for performing selection of a group stratification variable is configured in such a manner that group stratification variables to be analyzed through stratification are provided and the user is able to select (double-click) each of the variables.

Herein, the selection of a group stratification variable includes a stage of making selection when the user wants or of not making selection and enabling proceeding to the next step.

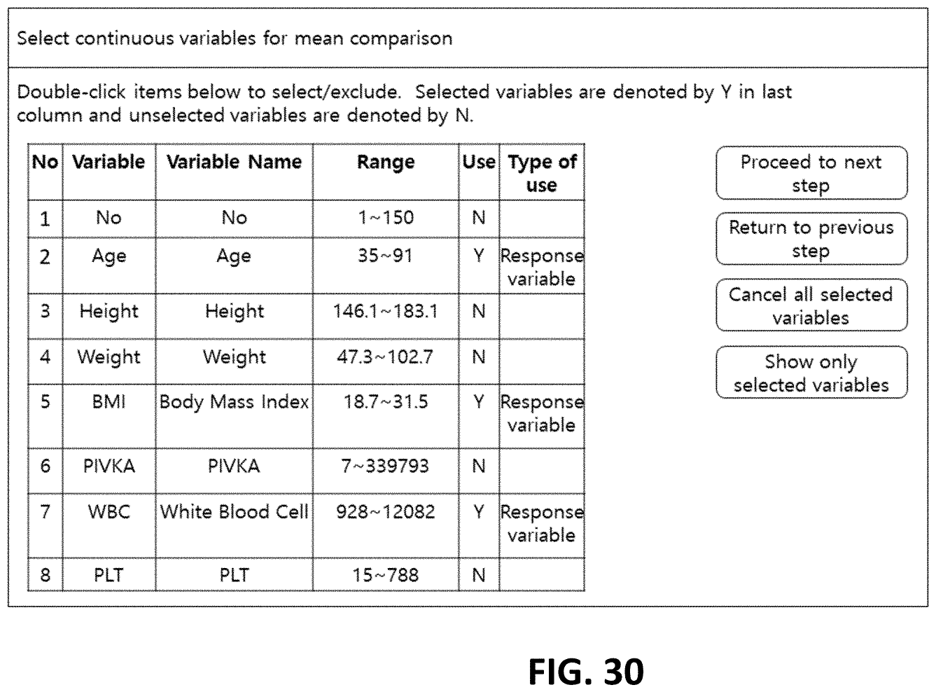

FIG. 30 illustrates an example of a user interface for performing selection of a continuous response variable, in the stage of setting variables and parameters.

The user interface for performing selection of a continuous response variable is configured in such a manner that continuous variables for mean comparison are provided and the user is able to select (double-click) each of the variables.

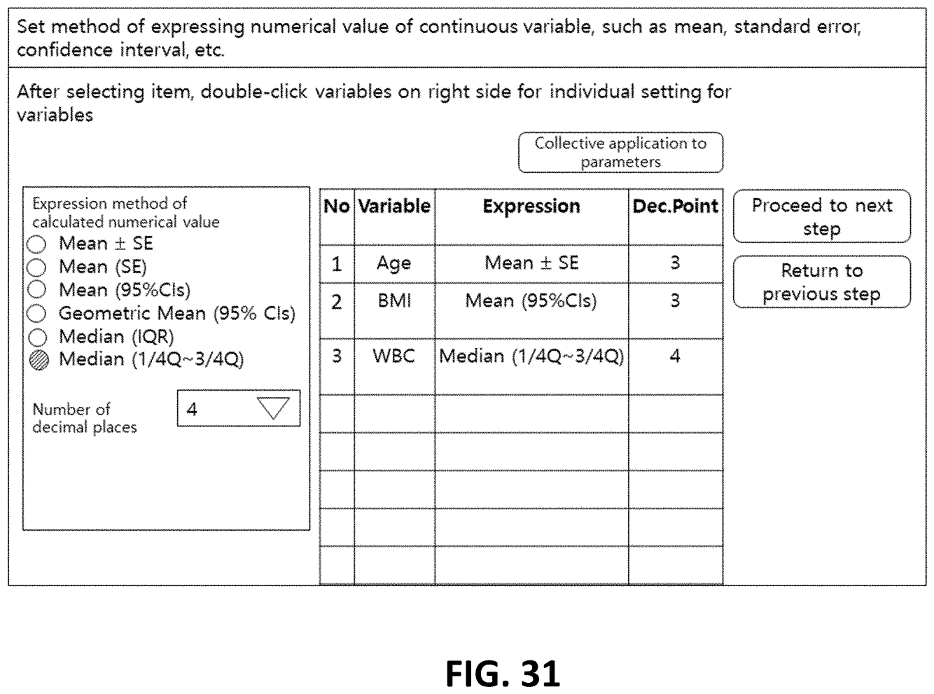

FIG. 31 illustrates an example of a user interface for performing selection of a continuous numerical value expression method, in the stage of setting variables and parameters.

The user interface for performing selection of a continuous numerical value expression method is to set a numerical value expression method for continuous variables, such as a mean, a standard error, a confidence interval, etc., and selection (double-click) is made for each of the variables to be set to different values.

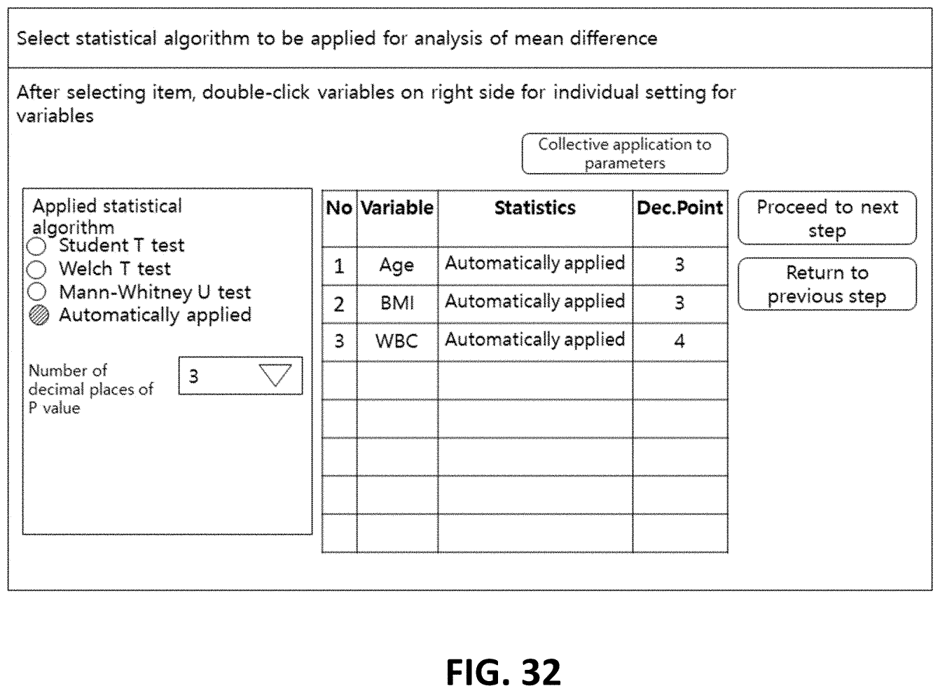

FIG. 32 illustrates an example of a user interface for performing selection of a particular algorithm, in the stage of setting variables and parameters.

The user interface for performing selection of a particular algorithm to be applied includes an item for manually selecting a statistical algorithm to be applied. The item includes an item for setting the number of decimal places of a P-Value. Next to the item, variables that may be set are provided. Collective application (a button for selecting collective application to parameters) to the variables is performed or the user makes selection (double-click) for individual setting for the variables.

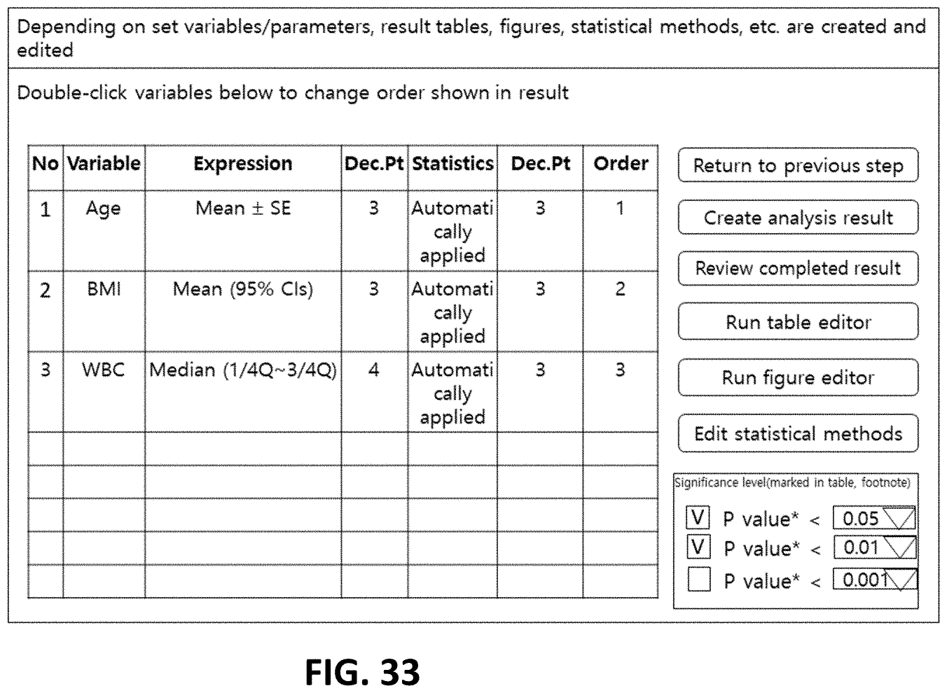

FIG. 33 illustrates an example of a user interface for performing selection of a result presentation method, in the stage of setting variables and parameters.

The user interface for performing selection of a result presentation method enables writing and editing a result table, a figure, a statistical method, etc. according to the set variables/parameters.

The variables to be shown in the result are displayed and are configured to possibly change the order of the variables. Next to this, the following are included: an item for creating an analysis result, an item for reviewing a completed result, an item for executing a table editor, an item for executing a figure editor, an item for a means for editing a statistical method, and an item for setting the number of digits of a significance level P-Value.

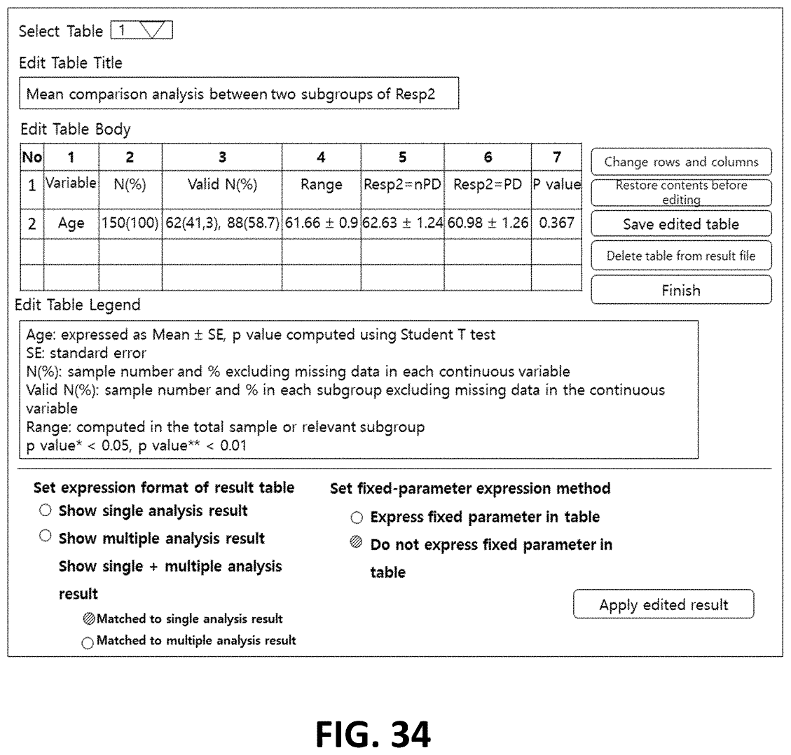

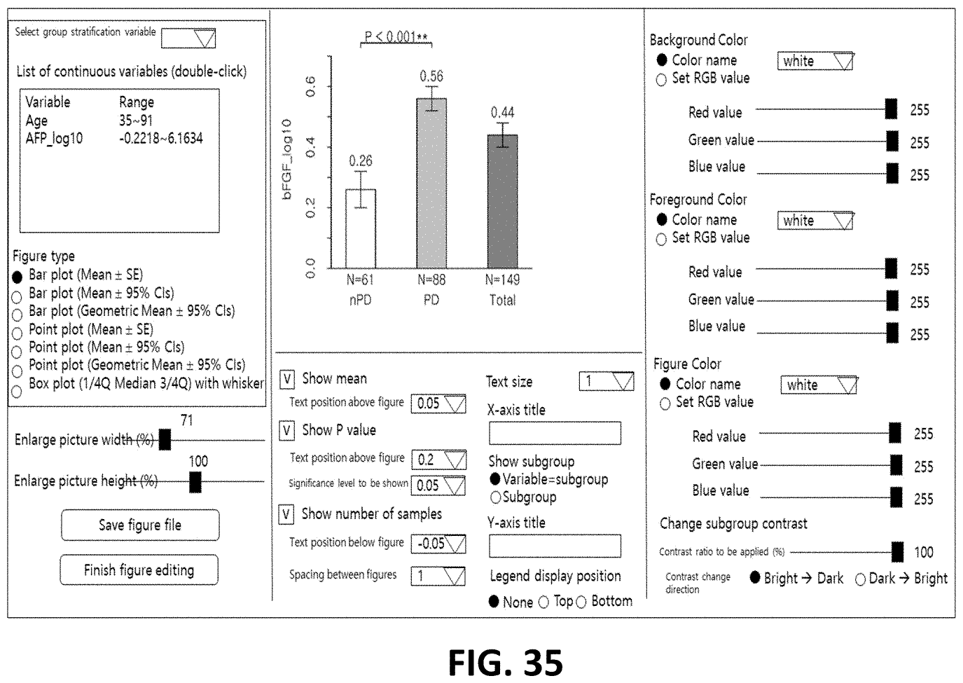

FIG. 34 is a diagram illustrating an example of a table editor, in the user interface for performing selection of a result presentation method as shown in FIG. 33. FIG. 35 is a diagram illustrating an example of a figure editor, in a user interface for performing selection of a result presentation method.

An analysis is performed according to the statistical algorithm selected through the above-described process, and pieces of generated result information are provided to the user according to the method set as shown in FIG. 33.

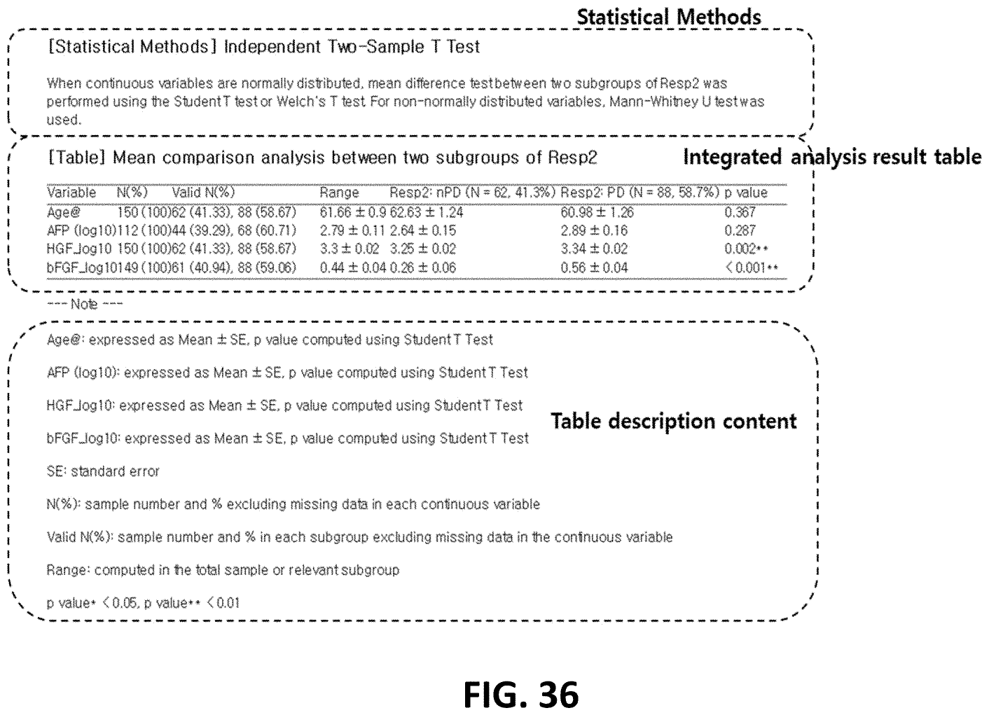

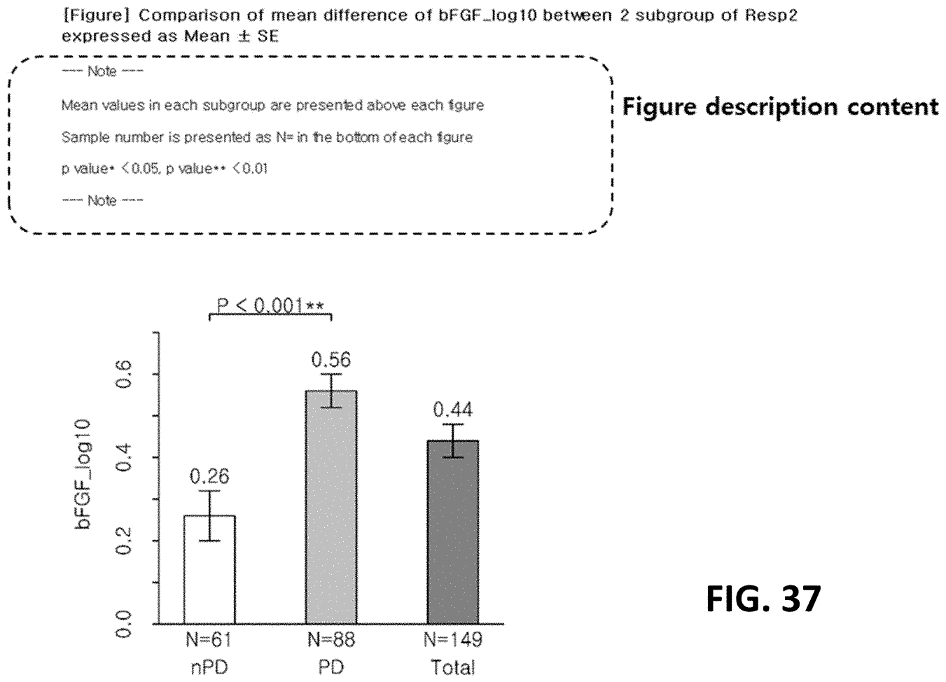

FIGS. 36 and 37 are diagrams illustrating such statistical analysis result information. FIG. 36 illustrates an integrated analysis result table, and FIG. 37 illustrates a graph that expresses an individual variable analysis result.

According to FIG. 36, an analysis result is provided as an integrated analysis result table, wherein the table for each variable is provided and explanatory information for the table is included.

According to FIG. 37, the individual variable analysis result is configured in such a manner that above the graph shown on a per-variable basis, an explanation of each graph is provided.

As described above, in the “statistical analysis using clinical data”, a statistical analysis execution stage for an analysis of a mean difference between groups of continuous variables (Part 4-1) has been described.

Part 4-2; a process for “an analysis of a factor influencing a continuous response variable”.

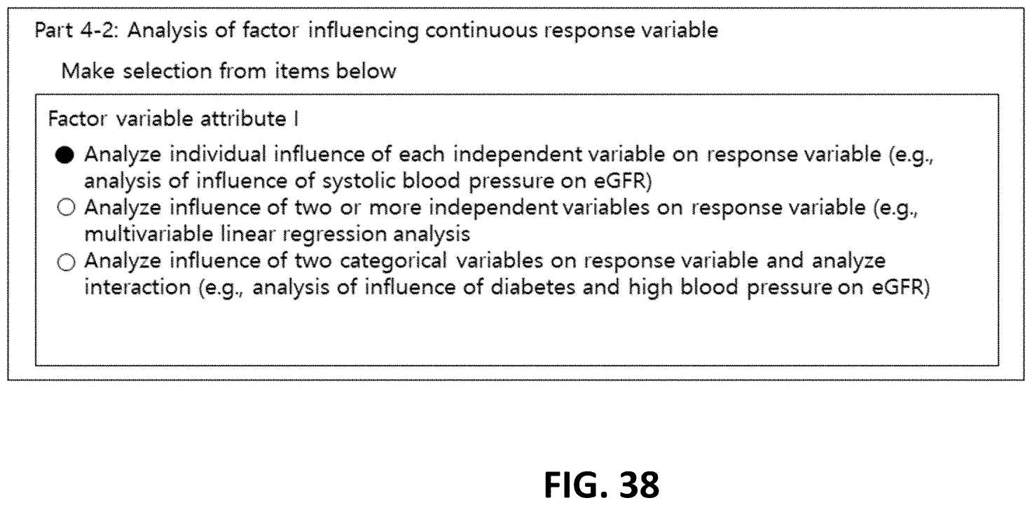

In the meantime, FIGS. 38 to 44 illustrate a process of the case in which among pieces of inquiry information as shown in FIG. 7, an analysis (Part 4-2) of a factor influencing a continuous response variable is selected as answer information in order to create a variable characteristic table for acquiring variable characteristic information in “a statistical analysis using clinical data”.

As shown in FIG. 38, when an analysis of a factor influencing a continuous response variable (Part 4-2) is selected, a process for selecting variable characteristics that are a combination of multiple conditions with respect to an analysis of a factor influencing a continuous response variable proceeds.

The analysis of a factor influencing a continuous response variable is composed of two pieces of inquiry information about factor variable attribute I.

The factor variable attribute I includes an analysis of individual influence of each independent variable on a response variable, an analysis of influence of two or more independent variables on a response variable, and an analysis of both influence of two categorical variables on a response variable and interaction.

Through this process, when the user's purpose of use of the program and the variable characteristic information are acquired, a statistical algorithm is selected accordingly.

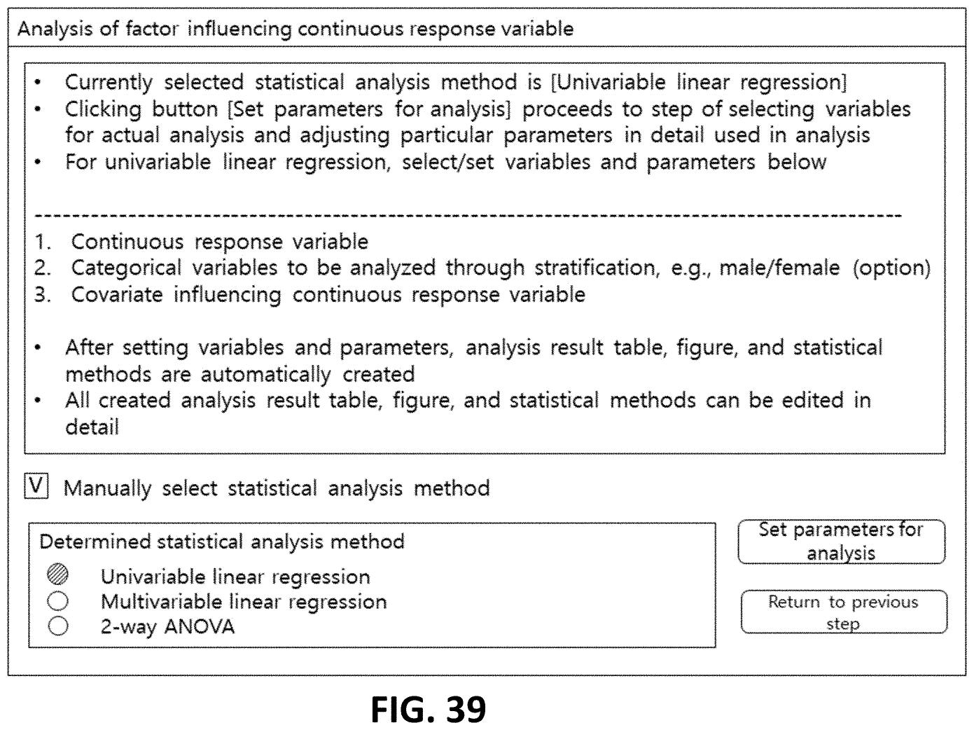

FIG. 39 illustrates a conversational interface for providing selection information for a statistical algorithm selected with respect to the analysis of a factor influencing a continuous response variable as described above.

The selection information includes variable and parameter setting information for making a statistical analysis execution stage proceed for the selected statistical algorithm, and further includes item information that enables the user to manually select a statistical analysis.

As shown in FIG. 39, a conversational interface is to provide selection information for a statistical algorithm. At the upper part of the conversational interface for selecting a specific statistical algorithm to be used for an analysis, current program use purpose information (an analysis of a factor influencing a continuous response variable) is displayed. In addition, inside the lower part, a statistical algorithm currently analyzed is displayed, and pieces of guide information to be selected/set for setting variables and parameters are included so that the statistical algorithm proceeds. A link button for setting variables and parameters, which is the next process according to the user's selection, is included.

In addition, an automatic/manual selection item for enabling the user to manually select a statistical algorithm is included, and statistical algorithm information that may be selected when manual selection is selected with respect to the selection item is further included.

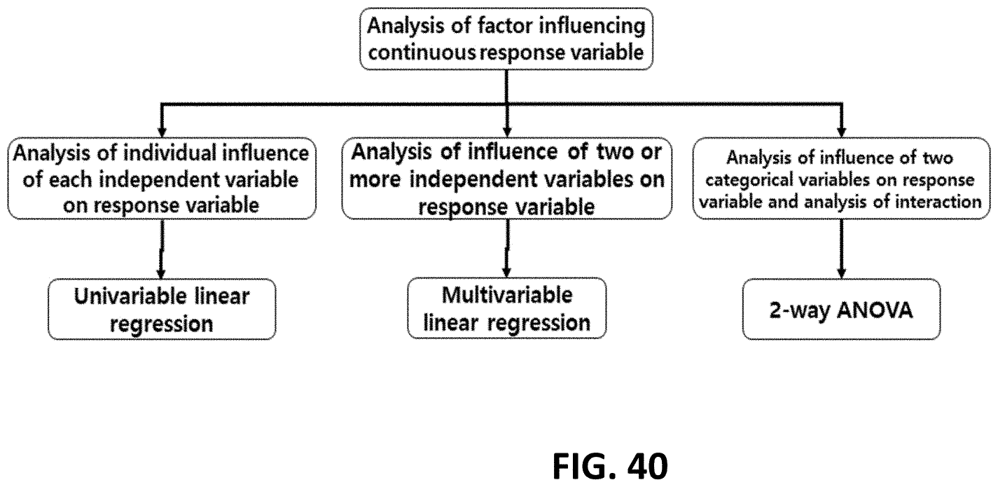

FIG. 40 is a flowchart illustrating a whole process for selecting a statistical algorithm according to variable characteristics in the variable characteristic information selected with respect to the purpose of use of the statistical analysis program. The process for selecting a statistical algorithm for an analysis of a factor influencing a continuous response variable is shown.

In the analysis of a factor influencing a continuous response variable, a statistical algorithm is selected through the following stages: determining a case of an analysis of individual influence of each independent variable on a response variable, determining a case of an analysis of influence of two or more independent variables on a variable, or determining a case of an analysis of both influence of two categorical variables on a response variable and interaction.

In the case of an analysis of individual influence of each independent variable on a response variable, statistical algorithm univariable linear regression is selected.

In the case of an analysis of influence of two or more independent variables on a response variable, multivariable linear regression is selected.

In the case of an analysis of both influence of two categorical variables on a response variable and interaction, 2-way ANOVA is selected.

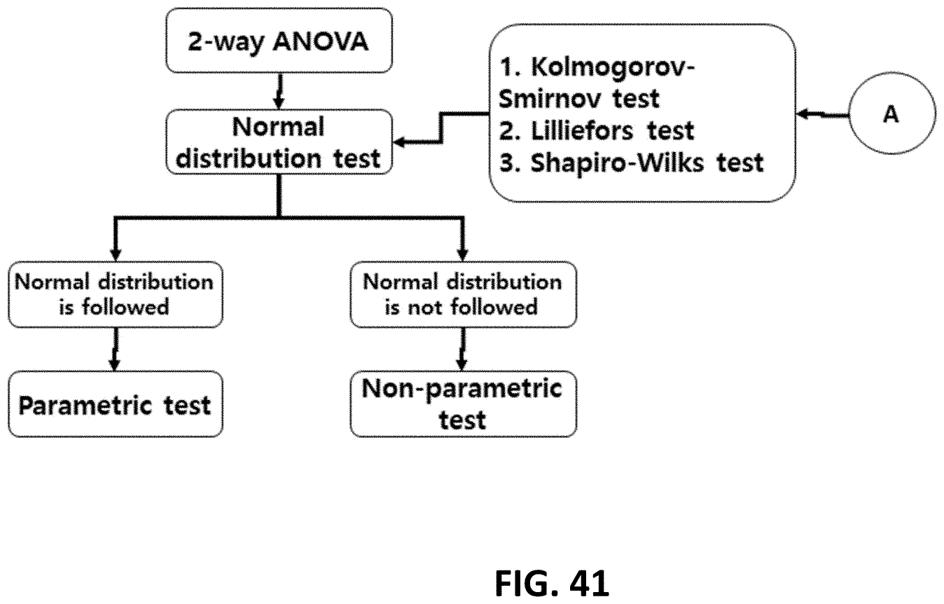

FIG. 41 is a flowchart illustrating a stage of automatically selecting a particular algorithm of algorithm “2-way ANOVA”.

The stage of automatically selecting a particular algorithm of 2-way ANOVA is composed of a stage of performing a normal distribution test to determine a case in which normal distribution is followed or a case in which normal distribution is not followed; and a stage of selecting a statistical algorithm according to a result of determination.

When normal distribution is followed, statistical algorithm “parametric test” is selected. When normal distribution is not followed, statistical algorithm “non-parametric test” is selected.

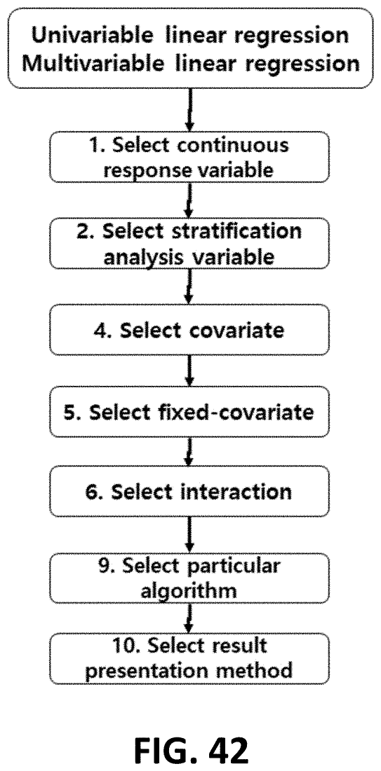

FIGS. 42 to 44 are diagrams illustrating a stage of setting, by a user, variables and parameters for each statistical algorithm through an interface.

FIG. 42 illustrates a stage of setting variables and parameters in the cases of “univariable linear regression” and “multivariable linear regression”.

“Univariable linear regression” and “multivariable linear regression” are composed of a stage of selecting a continuous response variable, a stage (option) of selecting a stratification analysis variable, a stage of selecting a covariate, a stage of selecting a fixed-covariate, a stage (option) of selecting interaction, a stage of selecting a particular algorithm, and a stage of selecting a result presentation method.

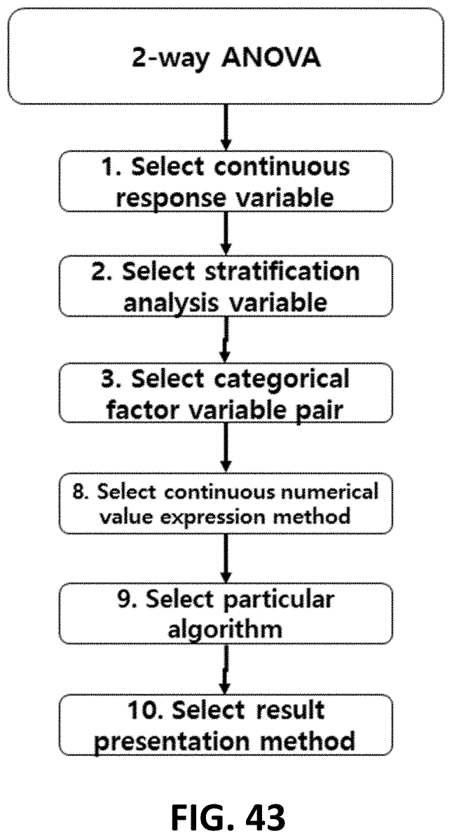

FIG. 43 illustrates a stage of setting variables and parameters in the case of “2-way ANOVA”.

“2-way ANOVA” is composed of a stage of selecting a continuous response variable, a stage (option) of selecting a stratification analysis variable, a stage of selecting a categorical factor variable pair, a stage of selecting a continuous numerical value expression method, a stage of selecting a particular algorithm, and a stage of selecting a result presentation method.

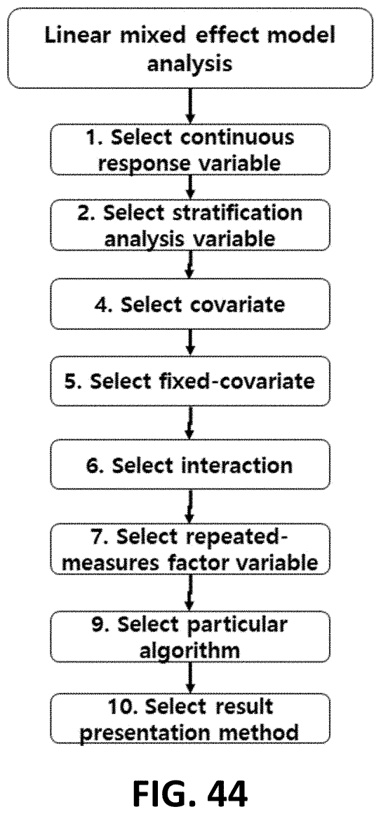

FIG. 44 illustrates a stage of setting variables and parameters in the case of “linear mixed effect model analysis”.

“Linear mixed effect model analysis” is composed of a stage of selecting a continuous response variable, a stage of selecting a stratification analysis variable, a stage (option) of selecting a covariate, a stage of selecting a fixed-covariate, a stage (option) of selecting interaction, a stage of selecting a repeated-measures factor variable, a stage of selecting a particular algorithm, and a stage of selecting a result presentation method.

Part 4-3; an automated process for “an analysis of association between categorical variables”.

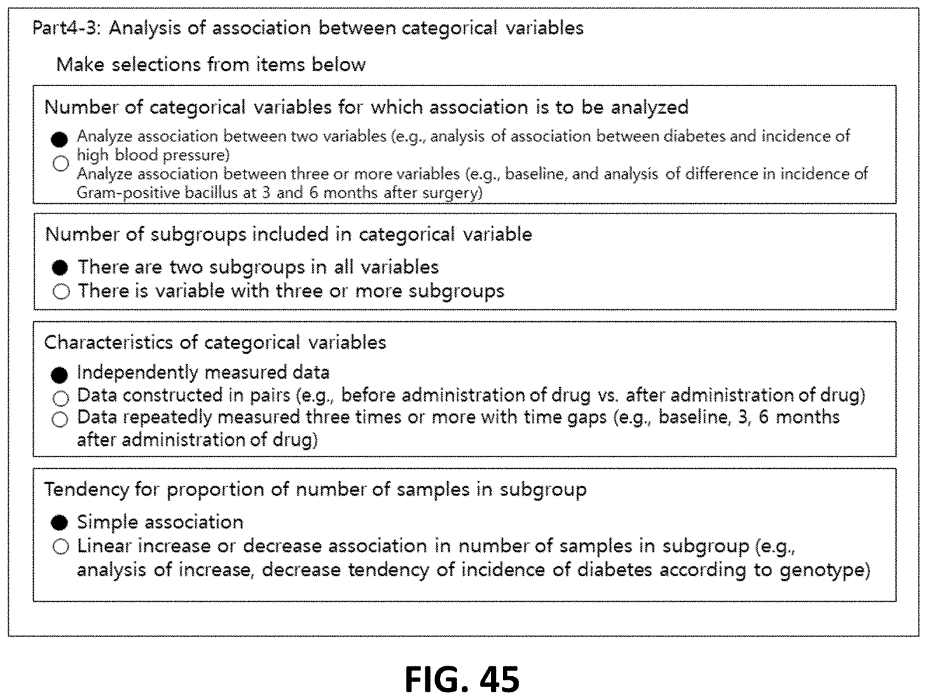

In the meantime, FIGS. 45 to 54 illustrate a process of the case in which among pieces of inquiry information as shown in FIG. 7, an analysis of association between categorical variables (Part 4-3) is selected as answer information in order to create a variable characteristic table for acquiring variable characteristic information in “an analysis of association between categorical variables”.

As shown in FIG. 45, when an analysis of association between categorical variables (Part 4-3) is selected, a process for selecting variable characteristics that are a combination of multiple conditions with respect to an analysis of association between categorical variables and to an analysis of a factor influencing a continuous response variable proceeds.

The analysis of association between categorical variables includes the number of categorical variables for which association is to be analyzed, the number of subgroups included in a categorical variable, the characteristics of categorical variables, and the tendency for the proportion of the number of samples in a subgroup.

Regarding the number of categorical variables for which association is to be analyzed, the following are included: an analysis of association between two variables, and an analysis of association between three or more variables.

Through this process, when the user's purpose of use of the program and the variable characteristic information are acquired, a statistical algorithm is selected accordingly.

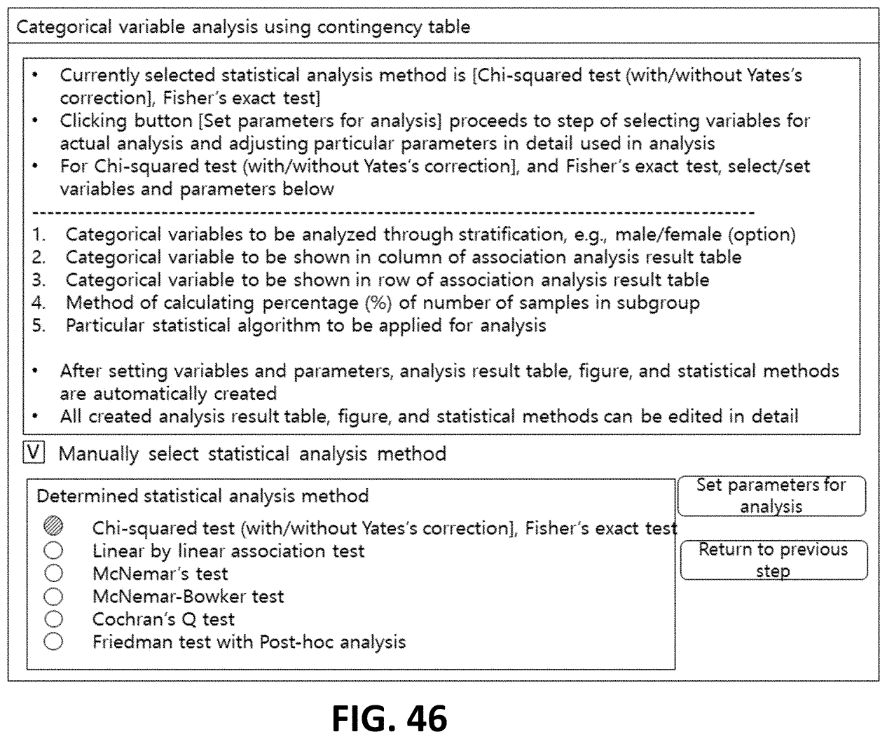

FIG. 46 illustrates a conversational interface for providing selection information for a statistical algorithm selected with respect to the analysis of association between categorical variables as described above.

The selection information includes variable and parameter setting information for making a statistical analysis execution stage proceed for the selected statistical algorithm, and further includes item information that enables the user to manually select a statistical analysis.

As shown in FIG. 46, a conversational interface is to provide selection information for a statistical algorithm. At the upper part of the conversational interface for selecting a specific statistical algorithm to be used for an analysis, current program use purpose information (a categorical variable analysis using a contingency table) is displayed. In addition, inside the lower part, a statistical algorithm currently selected is displayed, and pieces of guide information to be selected/set for setting variables and parameters are included so that the statistical algorithm proceeds. A link button for setting variables and parameters, which is the next process according to the user's selection, is included.

In addition, an automatic/manual selection item for enabling the user to manually select a statistical algorithm is included, and statistical algorithm information that may be selected when manual selection is selected with respect to the selection item is further included.

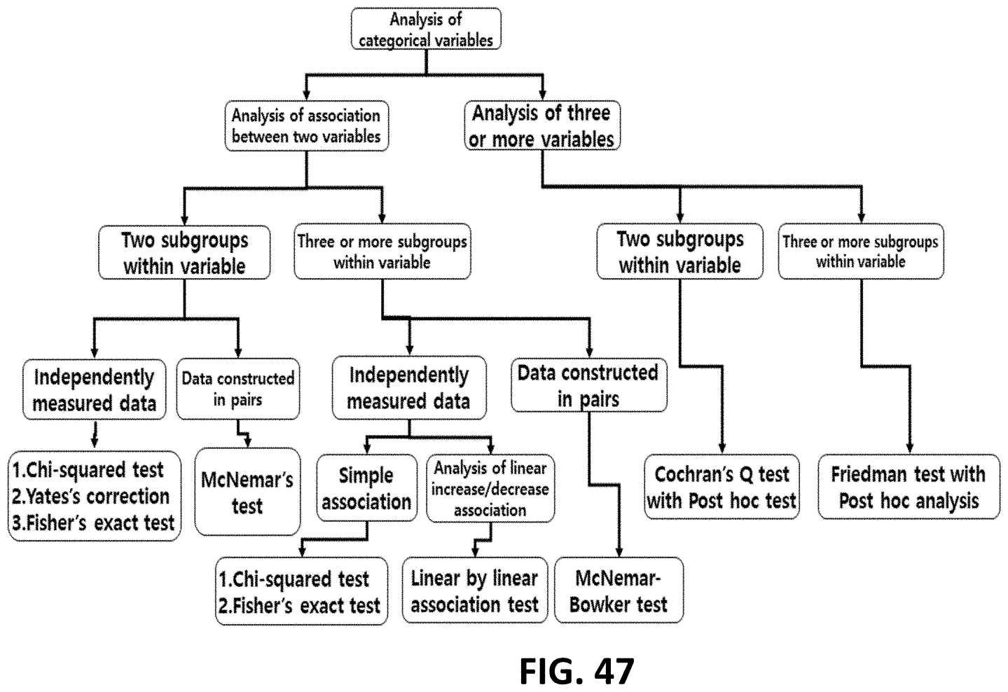

FIG. 47 is a flowchart illustrating a whole process for selecting a statistical algorithm according to variable characteristics in the variable characteristic information selected with respect to the purpose of use of the statistical analysis program. The process for selecting a statistical algorithm for an analysis of association between categorical variables is shown.

As shown in FIG. 47, a statistical algorithm is selected through the following stages: performing an analysis of a proportion difference between each of subgroups in one categorical variable; performing an analysis of association between two variables; or performing an analysis of three or more variables.

In the case of performing the analysis of association between two variables or the analysis of three or more variables, further included is a stage of determining a case in which there are two subgroups within a variable or a case in which there are three or more subgroups within a variable. For the analysis of association between two variables, included is a stage of selecting a statistical algorithm by determining a case in which data is independently measured or a case in which data is constructed in pairs.

In addition, in the analysis of association between two variables, in the case in which there are three or more subgroups within a variable and data is independently measured, further included is a stage of selecting a statistical algorithm by determining a case of a simple association or a case of an analysis of a linear increase/decrease association.

That is, with respect to an analysis of association between categorical variables, a process of selecting a statistical algorithm is composed of: a stage of performing an analysis of association between two variables or an analysis of three or more variables; and a stage of selecting a statistical algorithm by determining a case in which there are two subgroups within a variable or a case in which there are three or more subgroups within a variable. For the analysis of association between two variables, further included is a stage of selecting a statistical algorithm by determining a case in which data is independently measured or a case in which data is constructed in pairs. In the case in which there are three or more subgroups within a variable and data is independently measured, further included is a stage of selecting a statistical algorithm by determining a case of a simple association or a case of an analysis of a linear increase/decrease association.

In the case of an analysis of a proportion difference between each of subgroups in one categorical variable, statistical algorithm ‘one-sample proportion test’ is selected.

In the case in which an analysis of association between two variables is selected and there are two subgroups within a variable,

(a) For data independently measured, statistical algorithm Chi-squared test, Yates's correction, or Fisher's exact test is selected.

(b) For data constructed in pairs, statistical algorithm McNemar's test is selected.

In the case in which an analysis of association between two variables is selected and there are three subgroups within a variable,

(a) For data independently measured and a simple association, statistical algorithm Chi-squared test, or Fisher's exact test is selected.

(b) For data independently measured and an analysis of a linear increase/decrease association, statistical algorithm linear-by-linear association test is selected.

(c) For data constructed in pairs, statistical algorithm McNemar-Bowker test is selected.

In addition, in the case of the analysis of three or more variables,

(a) When there are two subgroups within a variable, Cochran's Q test with post hoc test is selected.

(b) When there are three or more subgroups within a variable, Friedman test with post hoc analysis is selected.

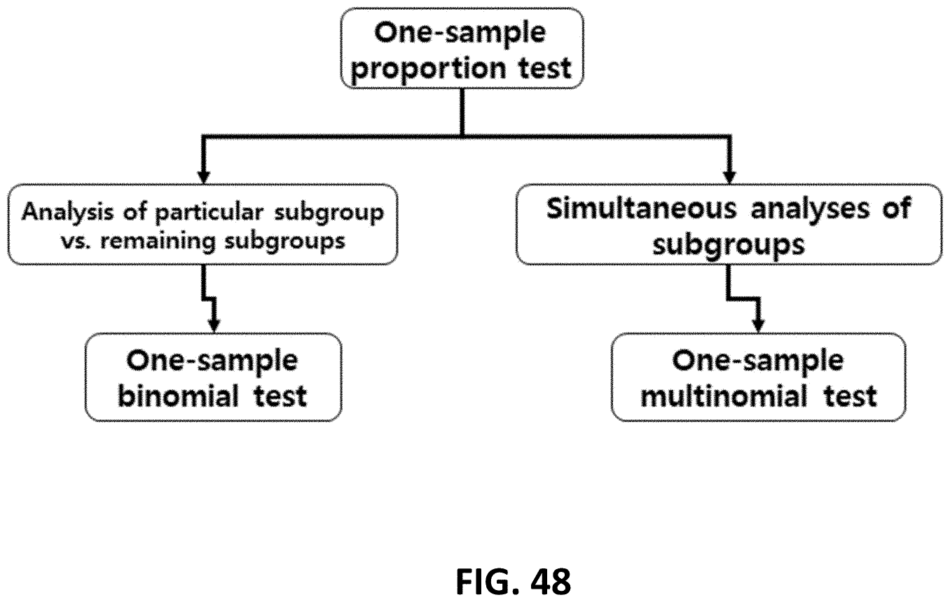

FIG. 48 is a flowchart illustrating a stage of automatically selecting a particular algorithm of statistical algorithm one-sample proportion test.

Regarding the stage of automatically selecting a particular algorithm of one-sample proportion test,

In the case of an analysis of a particular subgroup versus the remaining subgroups, statistical algorithm one-sample binomial test is selected. When subgroups are simultaneously analyzed together, one-sample multinomial test is selected.

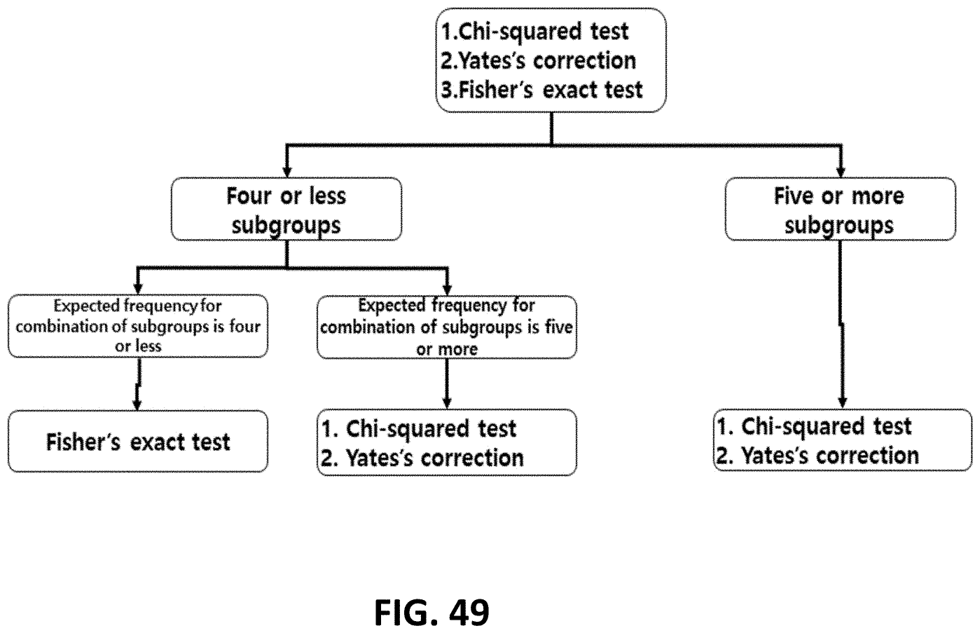

FIG. 49 is a flowchart illustrating a stage of automatically selecting a particular algorithm of statistical algorithms Chi-squared test, Yates's correction, and Fisher's exact test.

Included is a stage of selecting a statistical algorithm by determining a case in which the number of subgroups is four or less or a case in which the number of subgroups is five or more. In the case in which the number of subgroups is four or less, further included is a stage of determining a statistical algorithm by determining a case in which an expected frequency for a combination of subgroups is four or less or a case in which an expected frequency for a combination of subgroups is five or more.

When there are five or more subgroups, Chi-squared test, or Yates's correction is selected.

In the case in which the number of subgroups is four or less,

(a) When the expected frequency for a combination of subgroups is four or less, Fisher's exact test is selected.

(b) When the expected frequency for a combination of subgroups is five or more, Chi-squared test, or Yates's correction is selected.

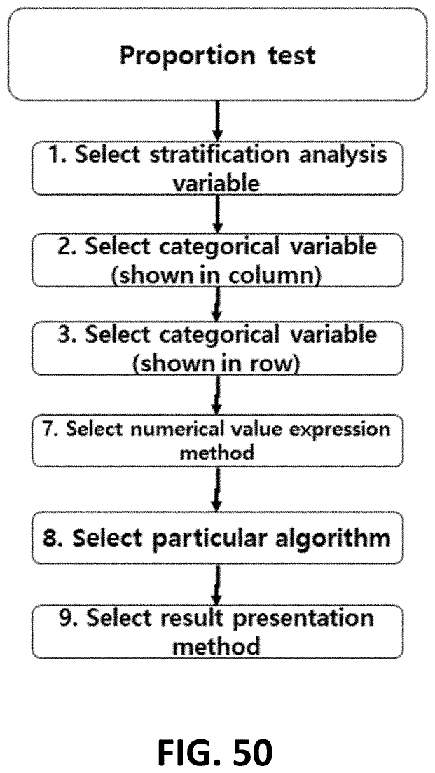

FIGS. 50 to 54 are diagrams illustrating a stage of setting, by a user, variables and parameters for each statistical algorithm through an interface.

FIG. 50 illustrates a stage of setting variables and parameters in the case of Proportion test.

Proportion test is composed of a stage (option) of selecting a stratification analysis variable, a stage of selecting a categorical variable (shown in a column), a stage (option) of selecting a categorical variable (shown in a row), a stage of selecting a numerical value expression method, a stage of selecting a particular algorithm, and a stage of selecting a result presentation method.

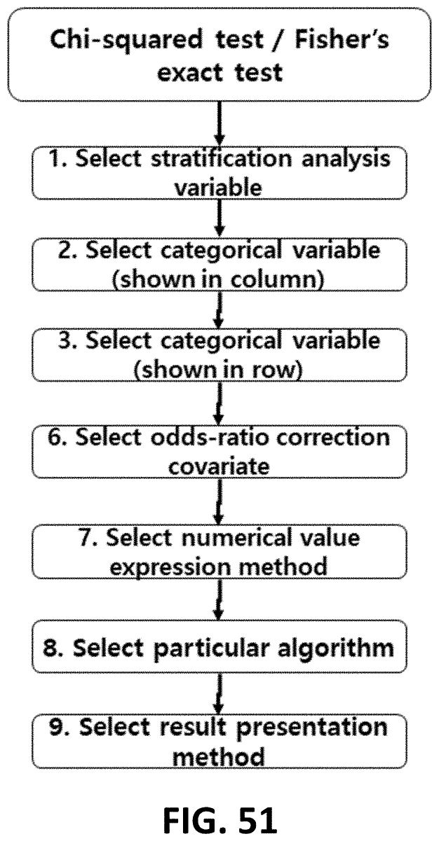

FIG. 51 illustrates a stage of setting variables and parameters in the cases of Chi-squared test, and Fisher's exact test.

Chi-squared test and Fisher's exact test are composed of a stage (option) of selecting a stratification analysis variable, a stage of selecting a categorical variable (shown in a column), a stage of selecting a categorical variable (shown in a row), a stage (option) of selecting an odds-ratio correction covariate, a stage of selecting a numerical value expression method, a stage of selecting a particular algorithm, and a stage of selecting a result presentation method.

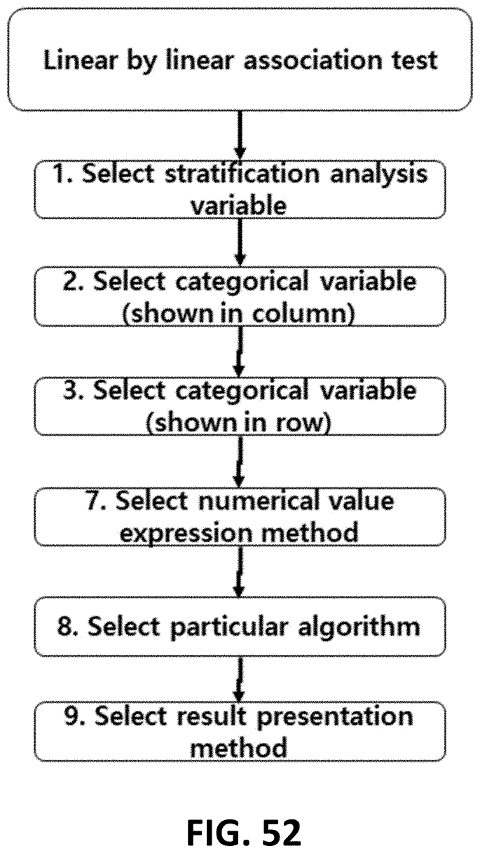

FIG. 52 illustrates a stage of setting variables and parameters in the case of linear-by-linear association.

Linear-by-linear association is composed a stage (option) of selecting a stratification analysis variable, a stage of selecting a categorical variable (shown in a column), a stage of selecting a categorical variable (shown in a row), a stage of selecting a numerical value expression method, a stage of selecting a particular algorithm, and a stage of selecting a result presentation method.

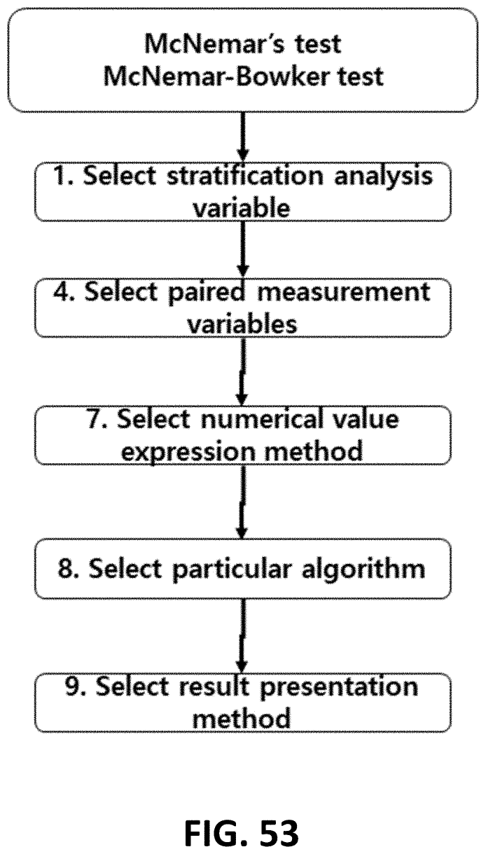

FIG. 53 illustrates a stage of setting variables and parameters in the cases of McNemar's test, and McNemar-Bowker test.

McNemar's test and McNemar-Bowker test are composed of a stage (option) of selecting a stratification analysis variable, a stage of selecting paired measurement variables, a stage of selecting a numerical value expression method, a stage of selecting a particular algorithm, and a stage of selecting a result presentation method.

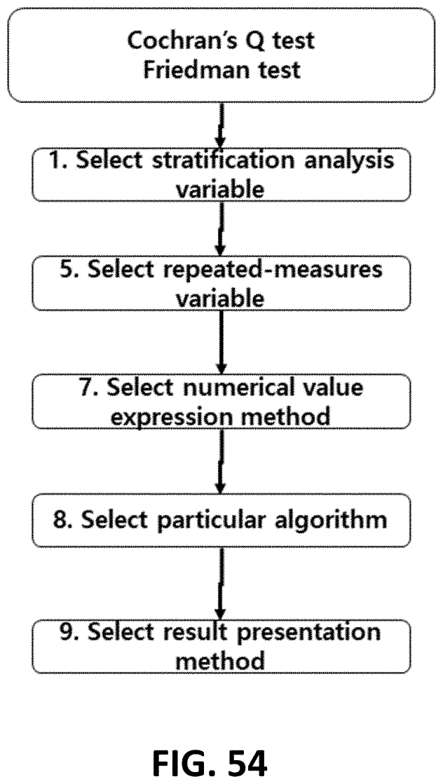

FIG. 54 illustrates a stage of setting variables and parameters in the cases of Cochran's Q test and Friedman test.

Cochran's Q test and Friedman test are composed of a stage (option) of selecting a stratification analysis variable, a stage of selecting paired measurement variables, a stage of selecting a numerical value expression method, a stage of selecting a particular algorithm, and a stage of selecting a result presentation method.

Part 4-4; an automated process for a factor analysis and development of a predictive model for categorical response prediction.

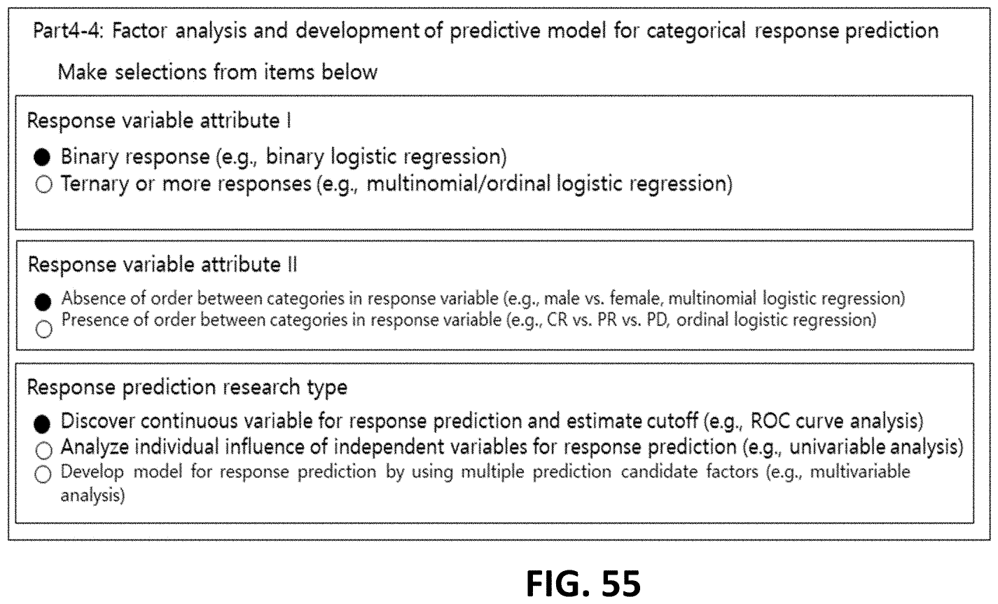

FIGS. 55 to 61 illustrate a process of the case in which among pieces of inquiry information as shown in FIG. 7, a factor analysis and development of a predictive model for categorical response prediction (Part 4-4) is selected as answer information in order to create a variable characteristic table for acquiring variable characteristic information in a factor analysis and development of a predictive model for categorical response prediction.

When a factor analysis and development of a predictive model for categorical response prediction (Part 4-4) is selected, a process for selecting variable characteristics that are a combination of multiple conditions with respect to a factor analysis and development of a predictive model for categorical response prediction proceeds as shown in FIG. 55.

The factor analysis and development of a predictive model for categorical response prediction is composed of inquiry information about a factor variable attribute, response variable attribute I, response variable attribute II, and a response prediction research type.

The response variable attribute I includes an item for identifying a response time.

The response variable attribute II includes an item for identifying whether there is order between categories included in a response variable.

The response prediction research type includes an item for finding a continuous variable for response prediction and measuring a cutoff, an item for analyzing individual influence of a variable, and an item for developing a predictive model.

Through this process, when the user's purpose of use of the program and the variable characteristic information are acquired, a statistical algorithm is selected accordingly.

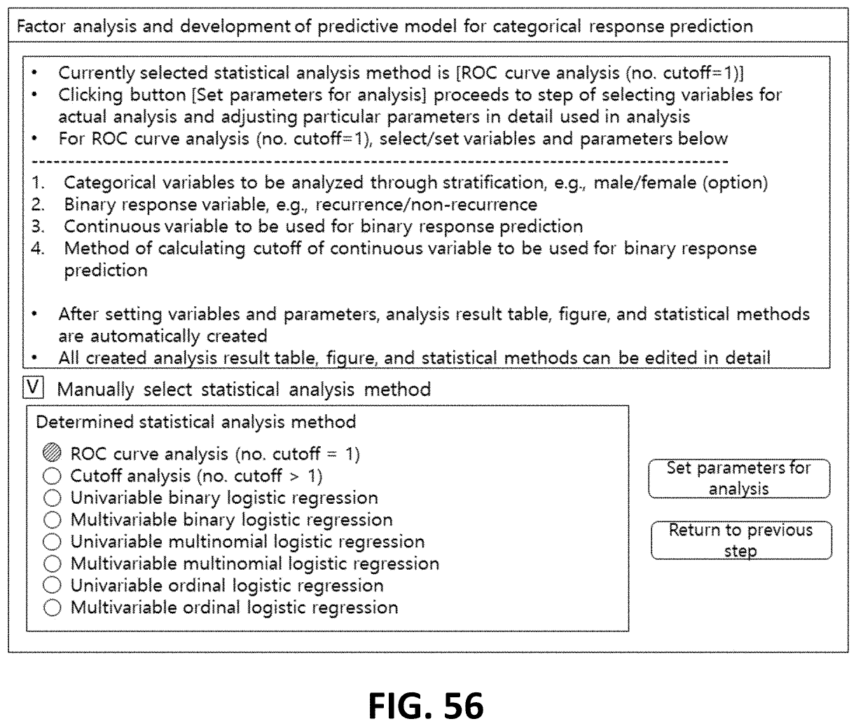

FIG. 56 illustrates a conversational interface for providing selection information for a statistical algorithm selected with respect to the analysis of association between categorical variables as described above.

The selection information includes variable and parameter setting information for making a statistical analysis execution stage proceed for the selected statistical algorithm, and further includes item information that enables the user to manually select a statistical analysis.

As shown in FIG. 56, a conversational interface is to provide selection information for a statistical algorithm.

At the upper part of the conversational interface for selecting a specific statistical algorithm to be used for an analysis, current program use purpose information, which is a factor analysis and development of a predictive model for categorical response prediction, is displayed. In addition, inside the lower part, a statistical algorithm currently selected is displayed, and pieces of guide information to be selected/set for setting variables and parameters are included so that the statistical algorithm proceeds. A link button for setting variables and parameters, which is the next process according to the user's selection, is included.

In addition, an automatic/manual selection item for enabling the user to manually select a statistical algorithm is included, and statistical algorithm information that may be selected when manual selection is selected with respect to the selection item is further included.

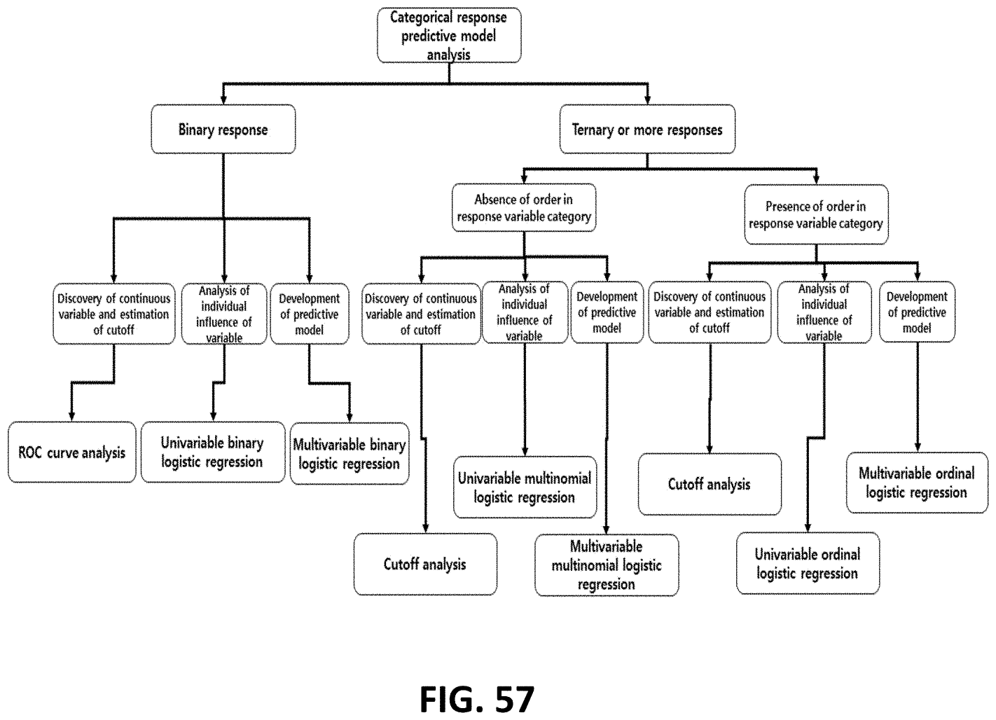

FIG. 57 is a flowchart illustrating a whole process for selecting a statistical algorithm according to variable characteristics in the variable characteristic information selected with respect to the purpose of use of the statistical analysis program. The process for selecting a statistical algorithm for a categorical response predictive model development analysis is shown.

As shown in FIG. 57, included is a stage of selecting a statistical algorithm by determining a case of a binary response or a case of a ternary or more responses.

In the case of the binary response, further included is a stage of selecting a statistical algorithm through a stage of performing the following: discovery of a continuous variable and estimation of a cutoff; an analysis of individual influence of a variable; or development of a predictive model. In the case of the ternary or more responses, further included is a stage of selecting a statistical algorithm through a stage of determining whether there is order in a response variable category or not, and for each of results of determining whether there is order in the response variable category or not, through a stage of performing the following: discovery of a continuous variable and estimation of a cutoff; an analysis of individual influence of a variable; or development of a predictive model.

Regarding selection of the statistical algorithm for the categorical response predictive model development analysis,

A statistical algorithm is selected by determining a case of a binary response or a case of a ternary or more responses, and by performing the following: discovery of a continuous variable and estimation of a cutoff; an analysis of individual influence of a variable; or development of a predictive model. Herein, in the case of the ternary or more responses, it is determined a case in which there is no order in a response variable category or a case in which there is order in a response variable category. For each of results thereof, further included is a stage of selecting a statistical algorithm by performing the following: discovery of a continuous variable and estimation of a cutoff; an analysis of individual influence of a variable; or development of a predictive model.

In the case of the binary response,

(a) As a statistical algorithm selected by performing discovery of a continuous variable and estimation of a cutoff, ROC curve analysis is selected.

(b) As a statistical algorithm selected by an analysis of individual influence of a variable, univariable binary logistic regression is selected.

(c) As a statistical algorithm selected by development of a predictive model, multivariable binary logistic regression is selected.

In the case of the ternary or more responses and absence of order in a response variable category,

(a) As a statistical algorithm selected by performing discovery of a continuous variable and estimation of a cutoff, cutoff analysis is selected.

(b) As a statistical algorithm selected by an analysis of individual influence of a variable, univariable multinomial logistic regression is selected.

(c) As a statistical algorithm selected by development of a predictive model multivariable multinomial logistic regression is selected.

In the case of the ternary or more responses and presence of order in a response variable category,

(a) As a statistical algorithm selected by performing discovery of a continuous variable and estimation of a cutoff, cutoff analysis is selected.

(b) As a statistical algorithm selected by an analysis of individual influence of a variable, univariable ordinal logistic regression is selected.

(c) As a statistical algorithm selected by development of a predictive model, multivariable ordinal logistic regression is selected.

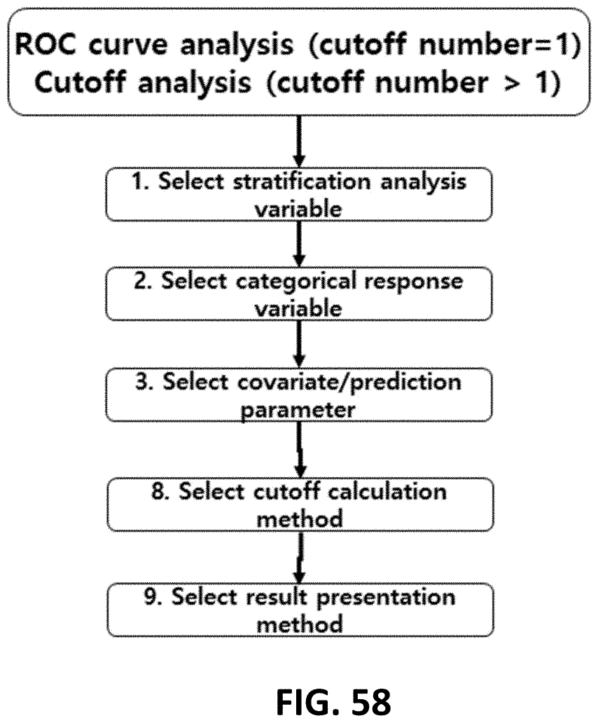

FIGS. 58 to 61 are diagrams illustrating a stage of setting, by a user, variables and parameters for each statistical algorithm through an interface.

FIG. 58 illustrates a stage of setting variables and parameters in the cases of ROC curve analysis (cutoff number=1) and cutoff analysis (cutoff number>1).

ROC curve analysis is composed of a stage (option) of selecting a stratification analysis variable, a stage of selecting a categorical variable, a stage of selecting a covariate/prediction parameter, a stage of selecting a cutoff calculation method, and a stage of selecting a result presentation method.

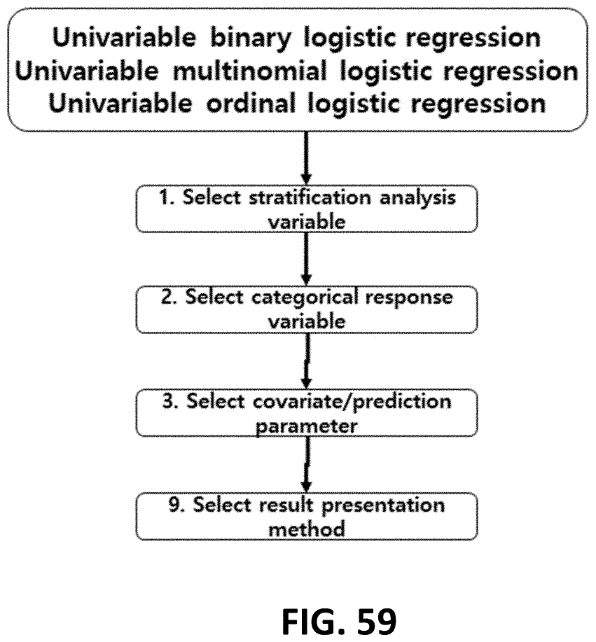

FIG. 59 illustrates a stage of setting variables and parameters in the cases of univariable binary logistic regression, univariable multinomial logistic regression, and univariable ordinal logistic regression.

Univariable binary logistic regression, univariable multinomial logistic regression, and univariable ordinal logistic regression are composed of a stage (option) of selecting a stratification analysis variable, a stage of selecting a categorical response variable, a stage of selecting a covariate/prediction parameter, and a stage of selecting a result presentation method.

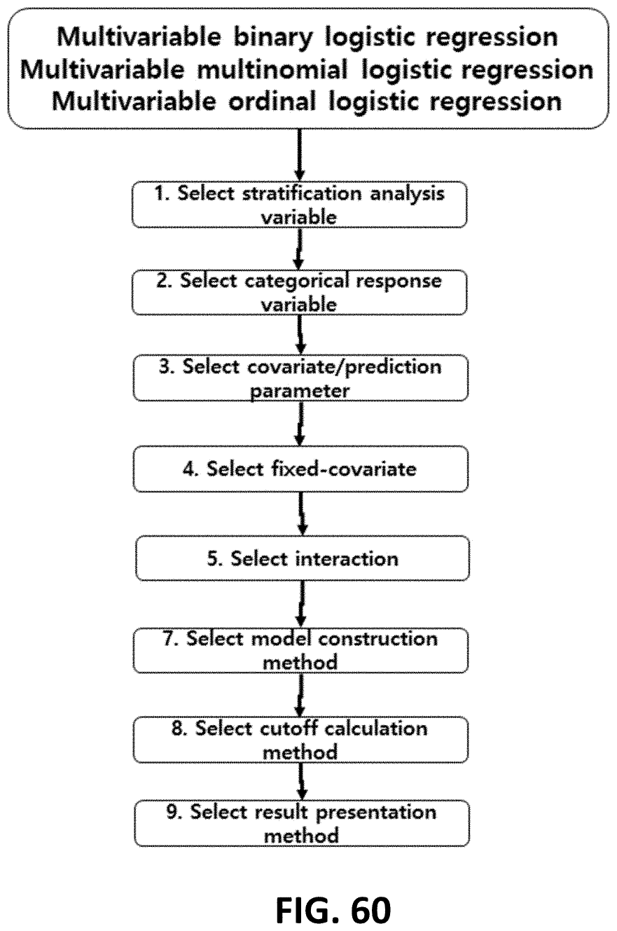

FIG. 60 illustrates a stage of setting variables and parameters in the cases of multivariable binary logistic regression, multivariable multinomial logistic regression, and multivariable ordinal logistic regression.

Univariable binary logistic regression, univariable multinomial logistic regression, and univariable ordinal logistic regression are composed of a stage (option) of selecting a stratification analysis variable, a stage of selecting a categorical response variable, a stage of selecting a covariate/prediction parameter, a stage (option) of selecting a fixed-covariate, a stage (option) of selecting interaction, a stage of selecting a model construction method, a stage of selecting a cutoff calculation method, and a stage of selecting a result presentation method.

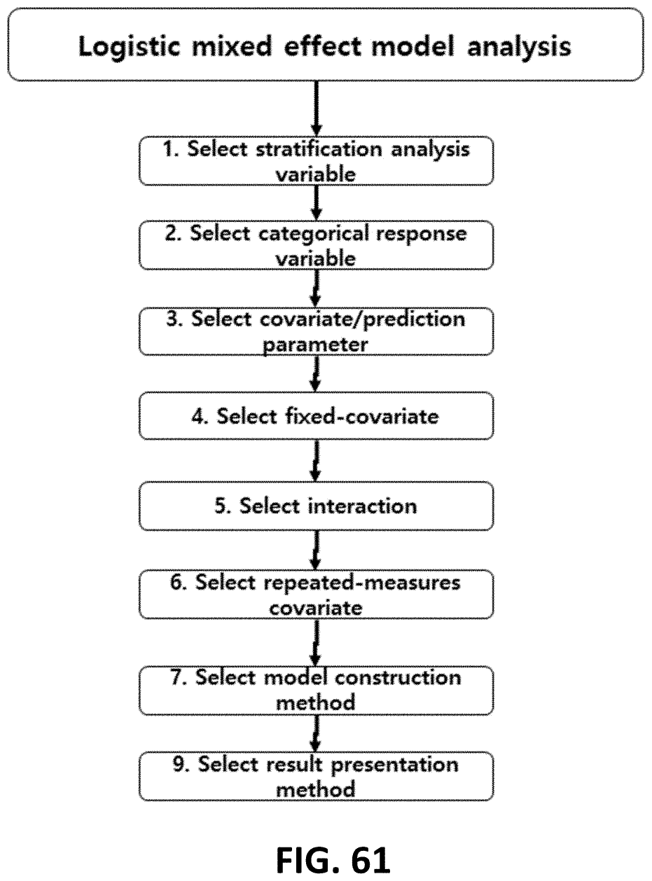

FIG. 61 illustrates a stage of setting variables and parameters in the case of logistic mixed effect model analysis.

Logistic mixed effect model analysis is composed of a stage (option) of selecting a stratification analysis variable, a stage of selecting a categorical response variable, a stage of selecting a covariate/prediction parameter, a stage (option) of selecting a fixed-covariate, a stage (option) of selecting interaction, a stage of selecting a repeated-measures covariate, a stage of selecting a model construction method, and a stage of selecting a result presentation method.

Part 4-5; an automated process for a survival data analysis.

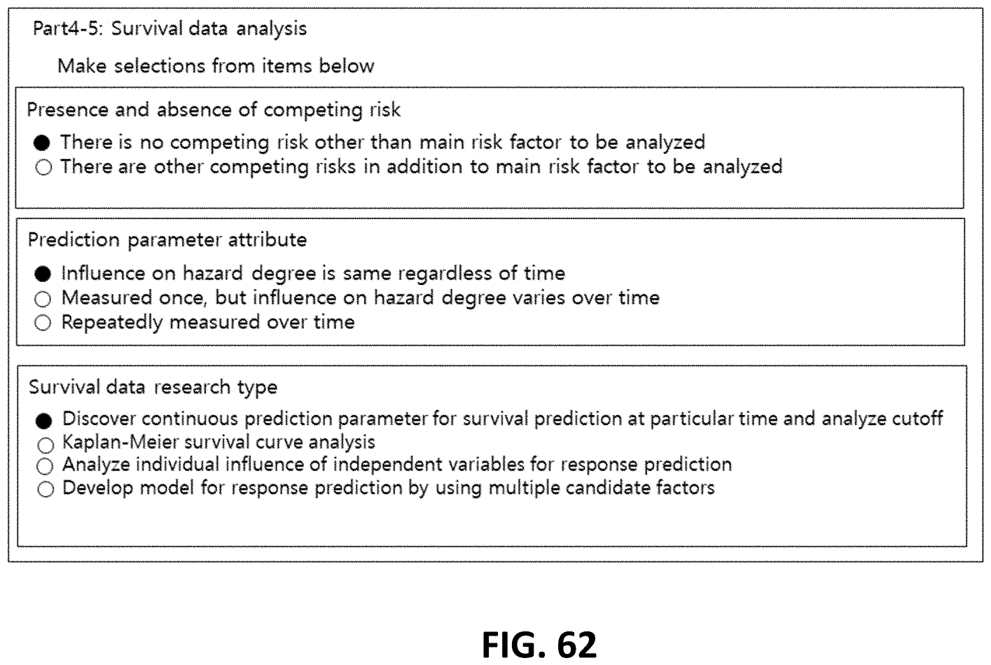

FIGS. 62 to 70 illustrates a process of the case in which among pieces of inquiry information as shown in FIG. 7, a survival data analysis (Part 4-5) is selected as answer information in order to create a variable characteristic table for acquiring variable characteristic information in a survival data analysis.

When the survival data analysis (Part 4-5) is selected, a process for selecting variable characteristics that are a combination of multiple conditions with respect to a survival data analysis proceeds as shown in FIG. 62.

The survival data analysis is composed of pieces of inquiry information about whether there is a competing risk, a prediction parameter attribute, and a survival data research type.

The prediction parameter attribute includes an item for identifying whether an influence on a hazard degree is the same regardless of time, an item for identifying whether there is a change of an influence on a hazard degree over time, and an item for identifying whether an influence on a hazard degree is repeatedly changed over time.

The survival data research type includes an item for discovery of a continuous prediction parameter and an analysis of a cutoff for survival prediction at a particular time, an item for Kaplan-Meier survival curve analysis, an item for an analysis of individual influence of independent variables for response prediction, and an item for development of a model for response prediction using multiple candidate factors.

Through this process, when the user's purpose of use of the program and the variable characteristic information are acquired, a statistical algorithm is selected accordingly.

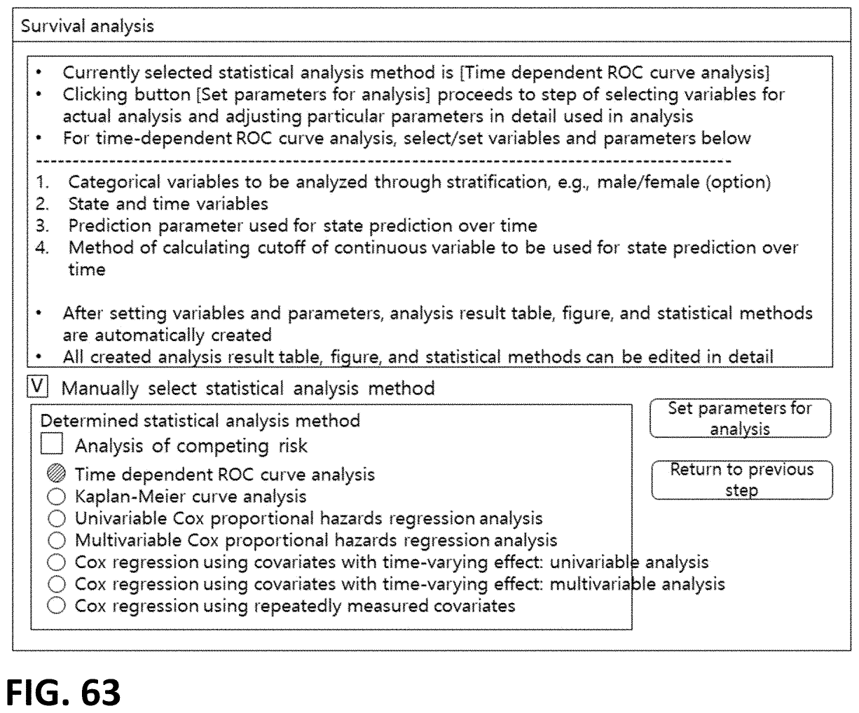

FIG. 63 illustrates a conversational interface for providing selection information for a statistical algorithm selected with respect to the survival data analysis as described above.

The selection information includes variable and parameter setting information for making a statistical analysis execution stage proceed for the selected statistical algorithm, and further includes item information that enables the user to manually select a statistical analysis.

As shown in FIG. 63, a conversational interface is to provide selection information for a statistical algorithm. At the upper part of the conversational interface for selecting a specific statistical algorithm to be used for an analysis, current program use purpose information is displayed. In addition, inside the lower part, a statistical algorithm currently selected is displayed, and pieces of guide information to be selected/set for setting variables and parameters are included so that the statistical algorithm proceeds. A link button for setting variables and parameters, which is the next process according to the user's selection, is included.

In addition, an automatic/manual selection item for enabling the user to manually select a statistical algorithm is included, and statistical algorithm information that may be selected when manual selection is selected with respect to the selection item is further included.

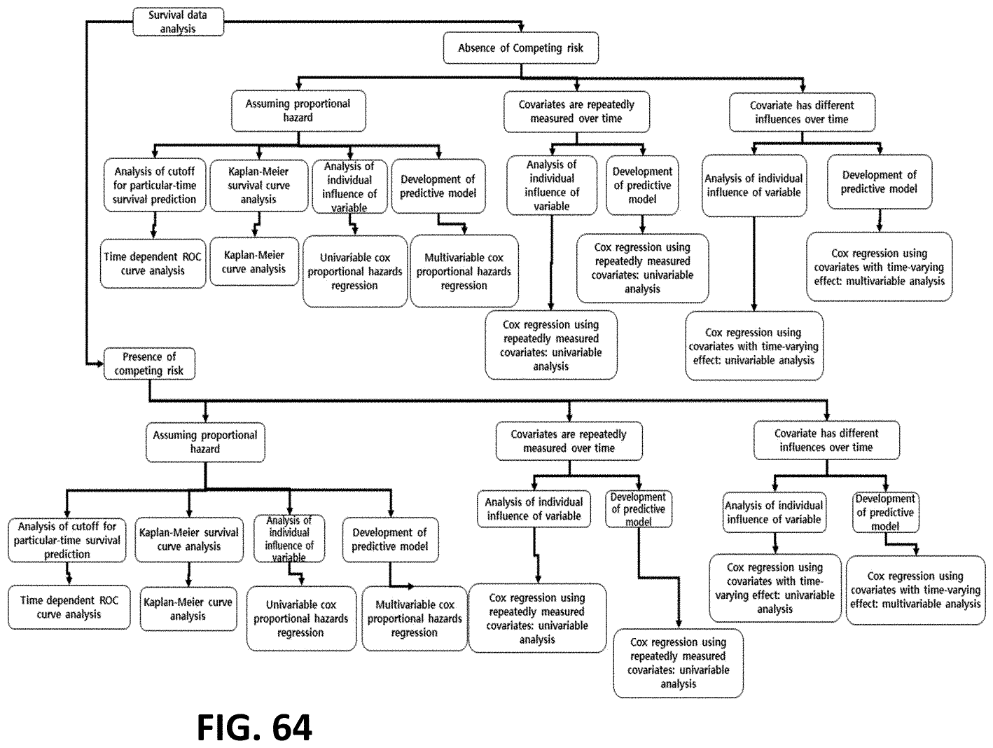

FIG. 64 is a flowchart illustrating a whole process for selecting a statistical algorithm according to variable characteristics in the variable characteristic information selected with respect to the purpose of use of the statistical analysis program. The process for selecting a statistical algorithm for a survival data analysis is shown.

As shown in FIG. 64, the following are included: a stage of determining whether there is a competing risk; a stage of determining, according to a result of determining whether there is a competing risk, a case of assuming each proportional hazard, a case in which covariates are repeatedly measured over time, or a case in which a covariate has different influences over time; and a stage of selecting a statistical algorithm with respect to the case of assuming each proportional hazard, the case in which covariates are repeatedly measured, or the case in which a covariate has different influences over time by performing the following: an analysis of a cutoff for particular-time survival prediction, Kaplan-Meier survival curve analysis, an analysis of individual influence of a variable, or an analysis of a predictive model.

Regarding selection of the statistical algorithm for the survival data analysis,

The following are included: a stage of determining whether there is a competing risk, and for presence and absence of the competing risk each, determining a case of assuming a proportional hazard, a case in which covariates are repeatedly measured over time, or a case in which a covariate has different influences over time; for the case of assuming a proportional hazard, a stage of selecting a statistical algorithm by performing an analysis of a cutoff for particular-time survival prediction, Kaplan-Meier survival curve analysis, an analysis of individual influence of a variable, or development of a predictive model; and for the case in which covariates are repeatedly measured over time or the case in which a covariate has different influences over time, a stage of selecting a statistical algorithm by performing an analysis of individual influence of a variable, or development of a predictive model.

Assuming a proportional hazard,

(a) By performing an analysis of a cutoff for particular-time survival prediction, statistical algorithm time-dependent ROC curve analysis is selected.

(b) By performing Kaplan-Meier survival curve analysis, statistical algorithm Kaplan-Meier curve analysis is selected.

(c) By performing an analysis of individual influence of a variable, statistical algorithm univariable Cox proportional hazards regression analysis is selected.

(d) By performing development of a predictive model, statistical algorithm multivariable Cox proportional hazards regression analysis is selected.

In the case in which covariates are repeatedly measured over time, statistical algorithm Cox regression using repeatedly measured covariates is selected by an analysis of individual influence of a variable and development of a predictive model.

In the case in which a covariate has different influences over time, statistical algorithm Cox regression using covariates with time-varying effect: univariable analysis is selected by an analysis of individual influence of a variable and development of a predictive model.

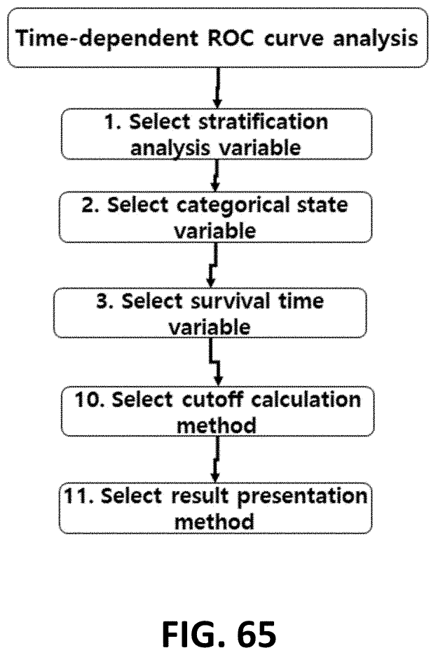

FIGS. 65 to 70 are diagrams illustrating a stage of setting, by a user, variables and parameters for each statistical algorithm through an interface.

FIG. 65 illustrates a stage of setting variables and parameters in the case of time-dependent ROC curve analysis.

Time-dependent ROC curve analysis is composed of a stage (option) of selecting a stratification analysis variable, a stage of selecting a categorical state variable, a stage of selecting a survival time variable, a stage of selecting a cutoff calculation method, and a stage of selecting a result presentation method.

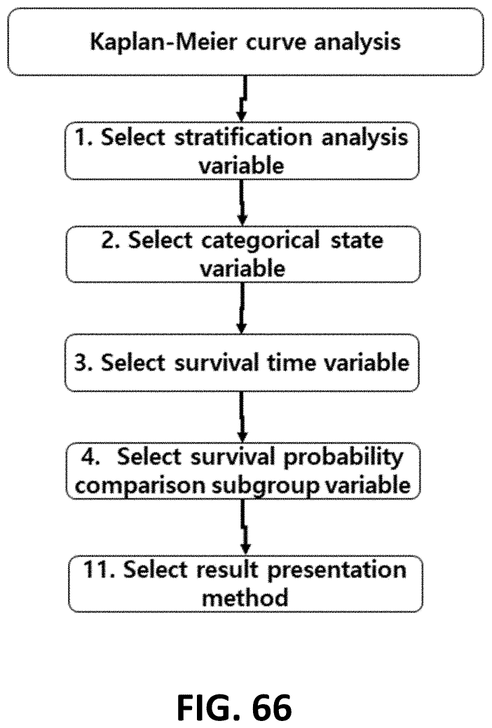

FIG. 66 illustrates a stage of setting variables and parameters in the case of Kaplan-Meier curve analysis.

Kaplan-Meier curve analysis is composed of a stage (option) of selecting a stratification analysis variable, a stage of selecting a categorical state variable, a stage of selecting a survival time variable, a stage (option) of selecting a survival probability comparison subgroup variable, and a stage of selecting a result presentation method.

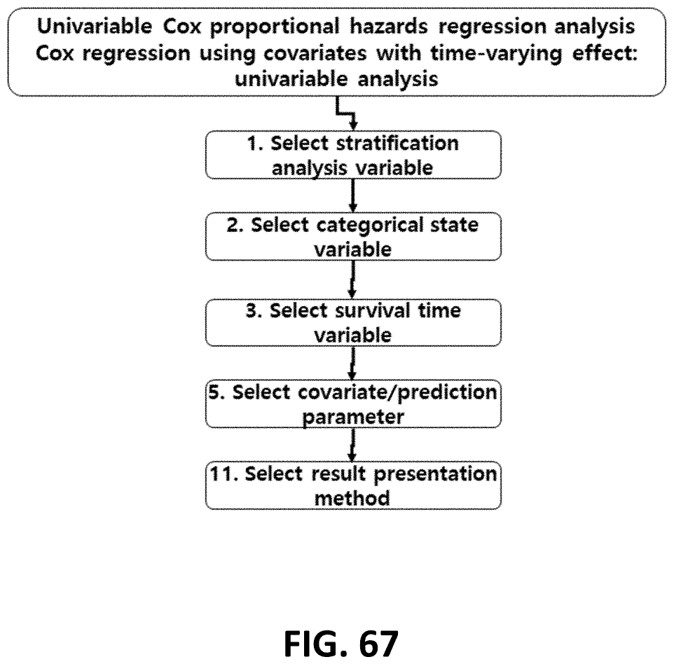

FIG. 67 illustrates a stage of setting variables and parameters in the cases of univariable Cox proportional hazards regression analysis, and Cox regression using covariates with time-varying effect: univariable analysis.

Univariable Cox proportional hazards regression analysis, and Cox regression using covariates with time-varying effect: univariable analysis are composed of a stage (option) of selecting a stratification analysis variable, a stage of selecting a categorical state variable, a stage of selecting a survival time variable, a stage of selecting a covariate/prediction parameter, and a stage of selecting a result presentation method.

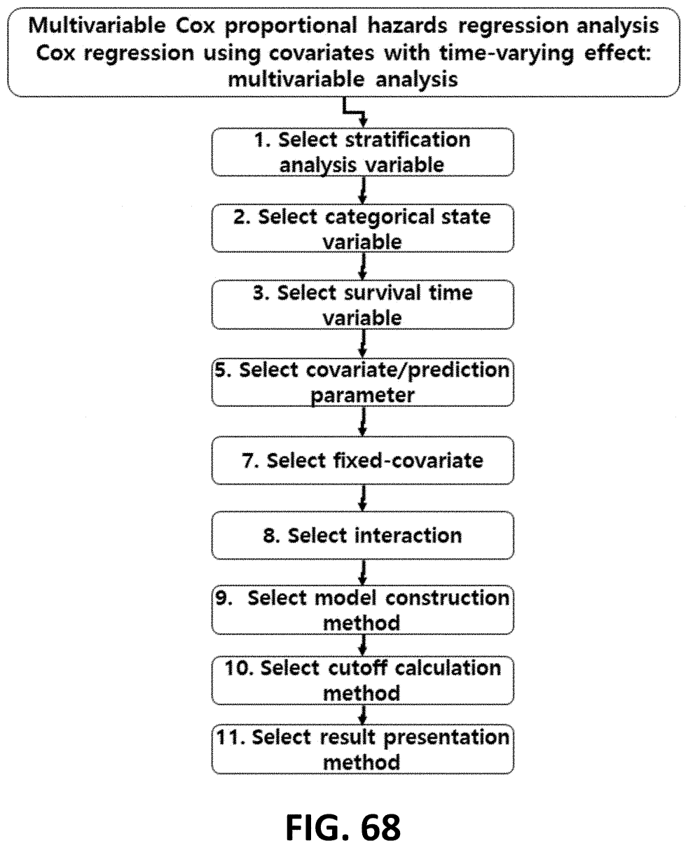

FIG. 68 illustrates a stage of setting variables and parameters in the cases of multivariable Cox proportional hazards regression analysis, and Cox regression using covariates with time-varying effect: multivariable analysis.

Multivariable Cox proportional hazards regression analysis, and Cox regression using covariates with time-varying effect: multivariable analysis are composed of a stage (option) of selecting a stratification analysis variable, a stage of selecting a categorical state variable, a stage of selecting a survival time variable, a stage of selecting a covariate/prediction parameter, a stage (option) of selecting a fixed-covariate, a stage (option) of selecting interaction, a stage of selecting a model construction method, a stage of selecting a cutoff calculation method, and a stage of selecting a result presentation method.

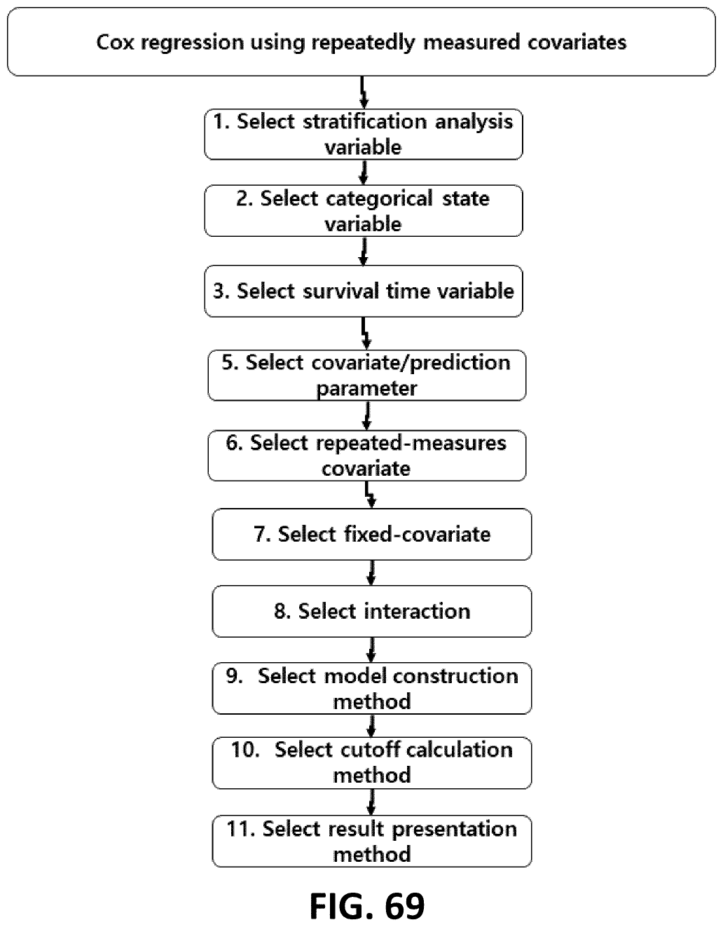

FIG. 69 illustrates a stage of setting variables and parameters in the case of Cox regression using repeatedly measured covariates.

Cox regression using repeatedly measured covariates is composed of a stage (option) of selecting a stratification analysis variable, a stage of selecting a categorical state variable, a stage of selecting a survival time variable, a stage of selecting a covariate/prediction parameter, a stage of selecting a repeated-measures covariate, a stage (option) of selecting a fixed-covariate, a stage (option) of selecting interaction, a stage of selecting a model construction method, a stage of selecting a cutoff calculation method, and a stage of selecting a result presentation method.

Part 4-6; an automated process for other analyses.

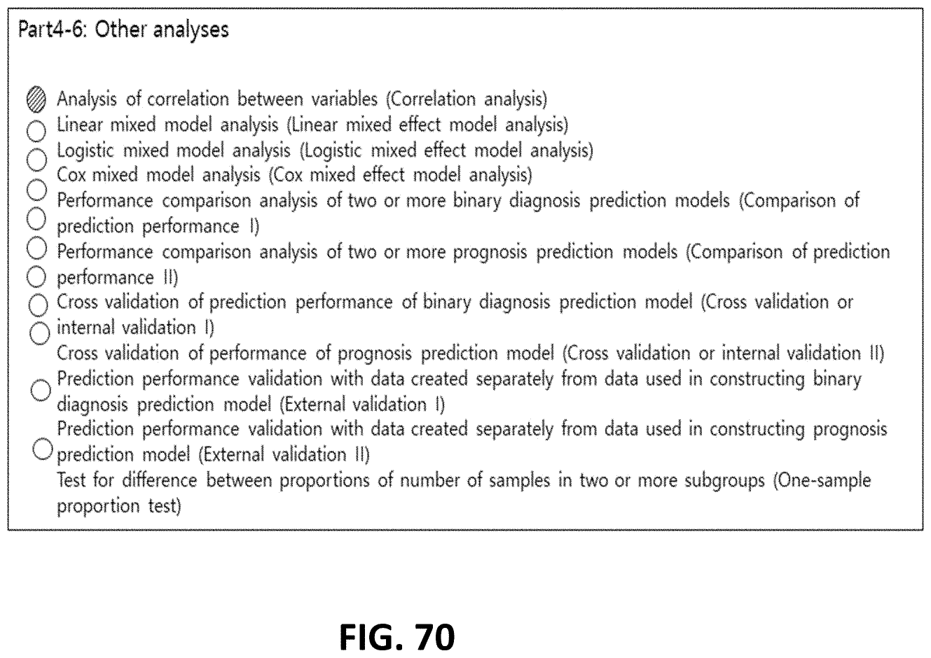

FIGS. 70 to 78 illustrates a process of the case in which among pieces of inquiry information as shown in FIG. 7, other analyses (Part 4-6) are selected as answer information in order to create a variable characteristic table for acquiring variable characteristic information in other analysis methods for manually selecting a particular analysis method and proceeding therewith.

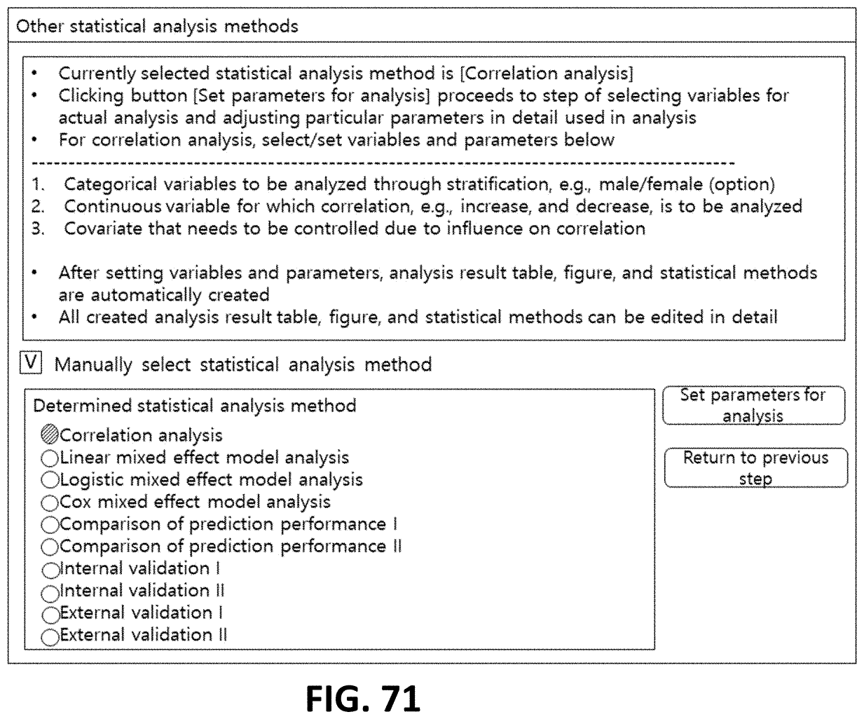

When the other analyses (Part 4-6) are selected, a process for the user to manually select a particular analysis method proceeds as shown in FIG. 71.

As shown in FIG. 70, the other analyses are composed of items for selecting particular analysis methods, and

The other analyses are composed of the following: an analysis of correlation between variables (correlation analysis), linear mixed model analysis (linear mixed effect model analysis), logistic mixed model analysis (logistic mixed effect model analysis), Cox mixed model analysis (Cox mixed effect model analysis), a performance comparison analysis of two or more binary diagnosis prediction models (comparison of prediction performance I), a performance comparison analysis of two or more prognosis prediction models (comparison of prediction performance II), cross validation of prediction performance of a binary diagnosis prediction model (cross validation or internal validation I), cross validation of performance of a prognosis prediction model (cross validation or internal validation II), prediction performance validation with data created separately from data used in constructing a prognosis prediction model (external validation I), prediction performance validation with data created separately from data used in constructing a prognosis prediction model (external validation II), and test for difference between proportions of the number of samples in two or more subgroups (one-sample proportion test).

FIG. 71 illustrates a conversational interface for providing selection information for the statistical algorithm selected as described above.

The selection information includes variable and parameter setting information for making a statistical analysis execution stage proceed for the selected statistical algorithm, and further includes item information that enables the user to reselect a statistical analysis.

As shown in FIG. 71, a conversational interface is to provide selection information for a statistical algorithm. The conversational interface for selecting a specific statistical algorithm to be used for an analysis displays a statistical algorithm currently selected, and pieces of guide information to be selected/set for setting variables and parameters are included so that the statistical algorithm proceeds. A link button for setting variables and parameters, which is the next process according to the user's selection, is included.

In addition, an automatic/manual selection item for enabling the user to reselect a statistical algorithm is included, and statistical algorithm information that may be selected when manual selection is selected with respect to the selection item is further included.

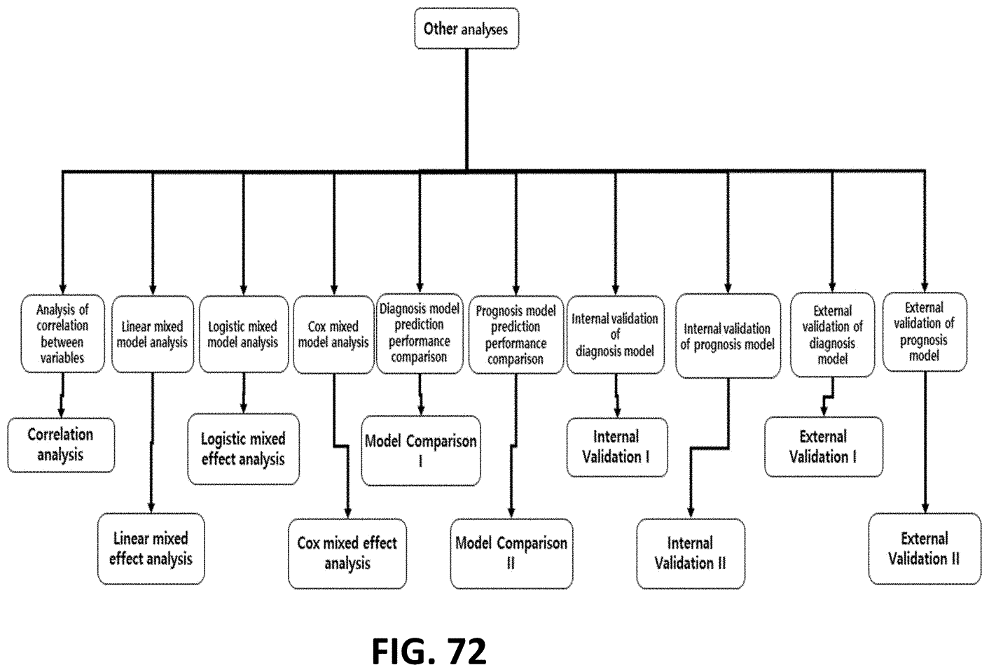

FIG. 72 is a flowchart illustrating a whole process for selecting a statistical algorithm according to variable characteristics in the variable characteristic information selected with respect to the purpose of use of the statistical analysis program. The process for selecting a statistical algorithm for other analyses is shown.

As shown in FIG. 72, the following are included: a stage of providing statistical analysis methods that the user can select, and a stage of selecting a statistical algorithm for the selected statistical analysis method.

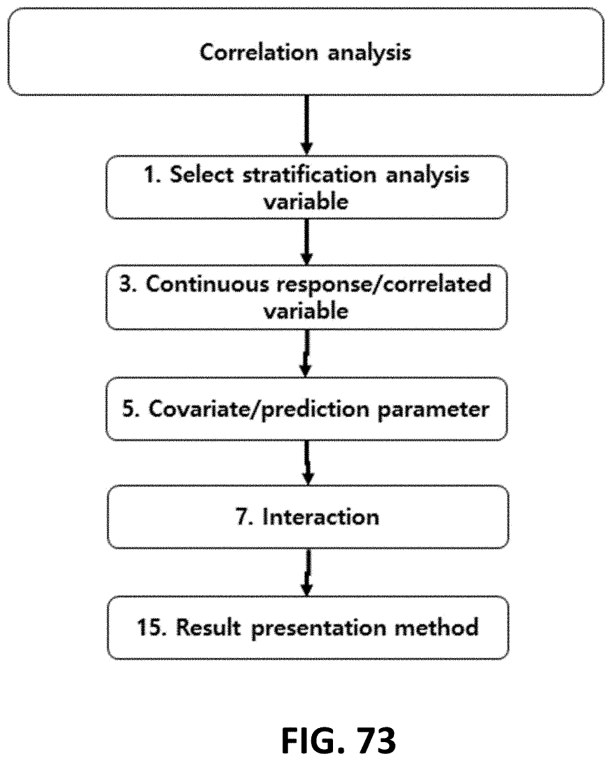

FIGS. 73 to 78 are diagrams illustrating a stage of setting, by a user, variables and parameters for each statistical algorithm through an interface.

FIG. 73 illustrates a stage of setting variables and parameters in the case of correlation analysis.

Correlation analysis is composed of a stage (option) of selecting a stratification analysis variable, a stage of selecting a continuous response/correlated variable, a stage of selecting a covariate/prediction parameter, a stage (option) of selecting interaction, and a stage of selecting a result presentation method.

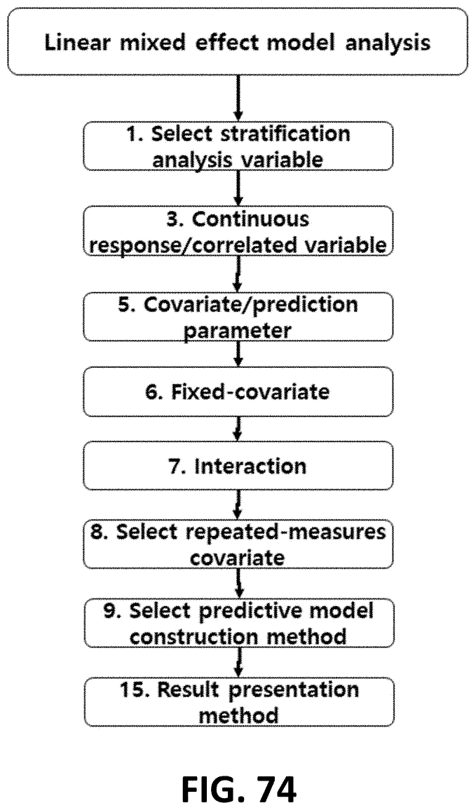

FIG. 74 illustrates a stage of setting variables and parameters in the case of linear mixed effect analysis.

Linear mixed effect analysis is composed of a stage (option) of selecting a stratification analysis variable, a stage of selecting a continuous response/correlated variable, a stage of selecting a covariate/prediction parameter, a stage (option) of selecting a fixed-covariate, a stage (option) of selecting interaction, a stage of selecting a repeated-measures covariate, a stage of selecting a predictive model construction method, and a stage of selecting a result presentation method.

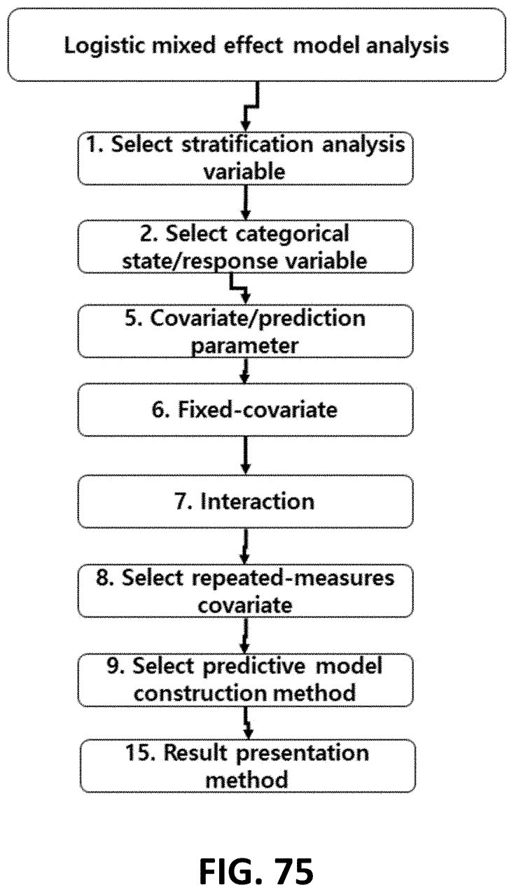

FIG. 75 illustrates a stage of setting variables and parameters in the case of logistic mixed effect analysis.

Logistic mixed effect analysis is composed of a stage (option) of selecting a stratification analysis variable, a stage of selecting a categorical state/response variable, a stage of selecting a covariate/prediction parameter, a stage (option) of selecting a fixed-covariate, a stage (option) of selecting interaction, a stage of selecting a repeated-measures covariate, a stage of selecting a predictive model construction method, and a stage of selecting a result presentation method.

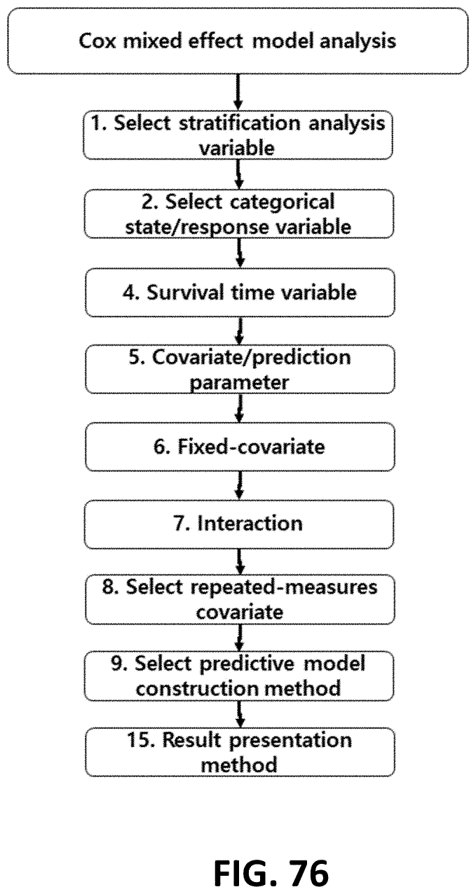

FIG. 76 illustrates a stage of setting variables and parameters in the case of Cox mixed effect analysis.

Cox mixed effect analysis is composed of a stage (option) of selecting a stratification analysis variable, a stage of selecting a categorical state/response variable, a stage of selecting a survival time variable, a stage of selecting a covariate/prediction parameter, a stage (option) of selecting a fixed-covariate, a stage (option) of selecting interaction, a stage of selecting a repeated-measures covariate, a stage of selecting a predictive model construction method, and a stage of selecting a result presentation method.

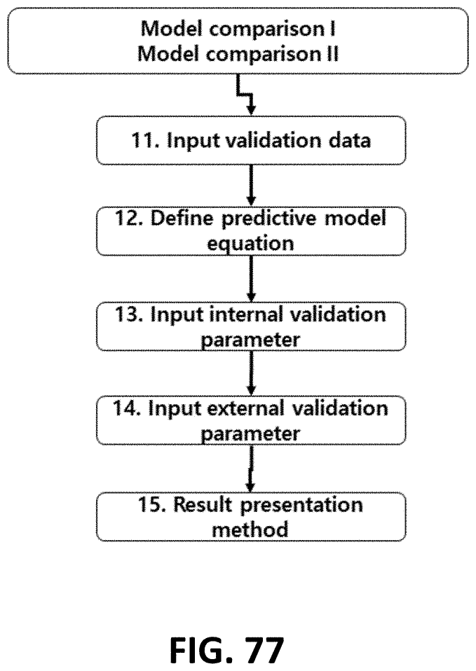

FIG. 77 illustrates a stage of setting variables and parameters in the cases of Model Comparison I, and Model Comparison II.

Model Comparison I and Model Comparison II are composed of a stage (option) of inputting validation data, a stage of defining a predictive model equation, a stage of inputting an internal validation parameter, a stage (option) of inputting an external validation parameter, and a stage of selecting a result presentation method.

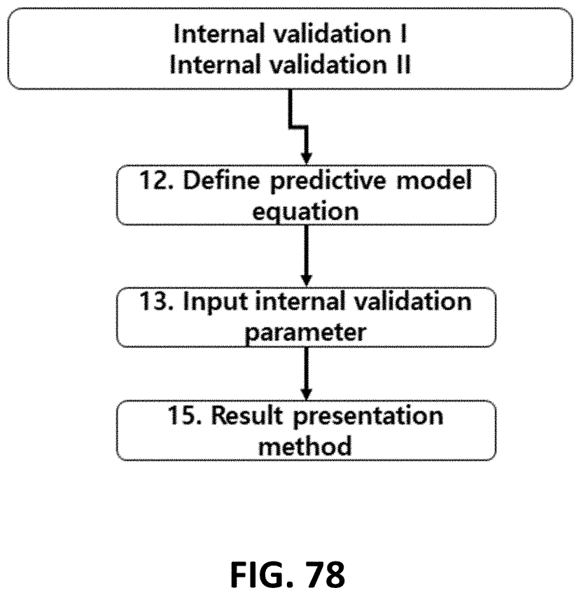

FIG. 78 illustrates a stage of setting variables and parameters in the cases of Internal Validation I, and Internal Validation II.

Internal Validation I and Internal Validation II are composed of a stage of defining a predictive model equation, a stage of inputting an internal validation parameter, and a stage of selecting a result presentation method.

FIG. 79 illustrates a stage of setting variables and parameters in the cases of External Validation I, and External Validation II.

External Validation I and External Validation II are composed of a stage of inputting validation data, a stage of defining a predictive model equation, a stage of inputting an external validation parameter, and a stage of selecting a result presentation method.

As described above, inquiry information/selection information is provided to a user so that a variable characteristic table is created according to the purpose of a statistical analysis and a statistical algorithm is selected according to the variable characteristics to perform the statistical analysis, whereby the user can easily select a statistical algorithm appropriate to the statistical analysis.

In the embodiment, Part 4 for statistical analyses using clinical data for which the user makes selection with respect to the purpose of a statistical analysis has been described. In addition, a statistical algorithm may be selected and a statistical analysis may be performed according to user's selection and the above-described process with respect to purposes of use of the program shown in FIG. 7; Part 1, Part 2, Part 3, a meta-analysis using an existing analysis result, and a reliability analysis of acquired data.

### INDUSTRIAL APPLICABILITY

According to the present disclosure, even when a user doesn't know specific statistical algorithms, data processing and a statistical analysis are effectively achieved through a conversational interface. Therefore, the present disclosure is a technology that can be widely used particularly in the field of statistical analyses for processing clinical data and can realize its practical and economical value.

1. A statistical analysis system using a conversational interface, the system comprising:
a conversational interface means (10) for providing inquiry information to a user to extract a user's purpose of a statistical analysis and variable information, and for collecting answer information provided from the user to inquiries;
an analysis feature information extraction means (20) for extracting analysis feature information by using the answer information of the user acquired by the conversational interface means (10);
an algorithm management means (30) for storing and managing statistical algorithms therein and for providing the statistical algorithms according to a request from a statistical analysis control means (40);
the statistical analysis control means (40) for controlling execution of the statistical analysis in such a manner that the conversational interface is provided to the user through the conversational interface means (10), the analysis feature information is extracted through the analysis feature information extraction means (20) from the answer information collected through the conversational interface, and the statistical algorithm to be used in the statistical analysis is selected according to the analysis feature information; and
a statistical analysis means (50) for performing the statistical analysis according to the statistical algorithm set by the statistical analysis control means (40) and for providing information about a result thereof.

2. The system of claim 1, wherein the analysis feature information extraction means (20) comprises: an answer information storage means (21) for collecting the answer information from the conversational interface means (10) and managing the answer information; a reference storage means (22) for storing and managing reference information therein for extraction of the analysis feature information for each piece of the answer information for the inquiry information; and a feature information extraction means (23) for extracting the analysis feature information from the stored answer information by referring to the reference information of the reference storage means (22).

3. The system of claim 1, wherein the statistical analysis control means (40) comprises: an algorithm registration means (41) for enabling a program producer or a system manager to additionally register and store a statistical algorithm in the algorithm management means (30) or to delete the statistical algorithms previously registered when necessary; an interface information management means (42) for storing and managing interface information therein for making conversation with the user to extract the analysis feature information and for receiving the answer information from the user; and an analysis control means (43) for providing the analysis feature information extraction means (20) with control information for extracting the analysis feature information from the answer information of the user so that the analysis feature information is extracted by the analysis feature information extraction means (20), and for providing control information to the statistical analysis means (50) so that the algorithm of the algorithm management means 30 is set from the analysis feature information extracted by the analysis feature information extraction means 20 and the statistical analysis is performed.

4. The system of claim 1, wherein the statistical analysis means (50) comprises: an analysis execution means (51) for executing the statistical analysis according to a process provided by the statistical algorithm selected by the statistical analysis control means (40); and an analysis result providing means (52) for providing analysis result information acquired through the analysis execution means (51), wherein the analysis result providing means (52) comprises an analysis result providing method setting means (52a) for enabling the user to set a method of providing the analysis result information.

5. The system of claim 1, wherein the statistical analysis control means (40) comprises:
a feature information extraction induction process means for controlling, when the statistical analysis system starts, the conversational interface means (10) for conversation with the user to provide the conversational interface, and for making the inquiries to the user through the conversational interface and acquiring and storing the answer information to the inquiries;
an analysis feature information extraction process means for controlling the analysis feature information extraction means (20) to extract the analysis feature information from the answer information;
an algorithm setting process means for selecting the algorithm matched to the analysis feature information extracted by the analysis feature information extraction stage (20) from the algorithm management means (30) and setting the selected algorithm in the statistical analysis means (50);
a statistical analysis execution process means for controlling the statistical analysis means (50) to execute the statistical analysis according to the set algorithm; and
a statistical analysis result providing process means for providing results acquired through a statistical analysis execution process to the user through the conversational interface means (10).

6. (canceled)

7. The system of claim 1, wherein the feature information extraction induction process means comprises: a purpose inquiry process means for making, when the statistical analysis system starts, a request to start a conversation analysis, and for providing, when a request to start the conversation analysis is made from the user, the user with an inquiry to acquire the purpose of use of a program and receiving an answer thereto; and a variable characteristic information inquiry process means for storing, when the answer is received through the purpose inquiry process means, the answer in the analysis feature information extraction means and for providing the inquiry information to create a variable characteristic table corresponding to the user's selected purpose of the statistical analysis, and for receiving the answer information and storing the answer information in the analysis feature information extraction means.

8. The system of claim 7, wherein the inquiry information provided by the purpose inquiry process means to the user to acquire the purpose of the statistical analysis includes an analysis required for making a research plan, sub-data extraction for a research from raw data and merging of different pieces of data, clinical data preprocessing for a statistical analysis, a statistical analysis using clinical data, a meta-analysis using an existing analysis result, and a reliability analysis of acquired data.

9. The system of claim 1, wherein the conversational interface provided by the conversational interface means (10) has an upper part in which bookmarks for respective inquiry stages (Parts) to proceed according to a process are provided, a lower part in which the inquiry information for each step is provided, and a part of a right side in which a link mark for proceeding from a current step to a next step is provided, and
under the answer information that is selected for the inquiry information in each of the inquiry stages, example information is provided so that the user is able to easily select the answer information, that is, an answer to the inquiry information.

10. The system of claim 1, wherein when the purpose of the statistical analysis acquired through the purpose inquiry analysis process means is a statistical analysis using clinical data (Part 4), the inquiry information provided for creating the variable characteristic table corresponding to the user's purpose of the statistical analysis by the analysis feature information extraction means (20) includes an analysis of a mean difference between groups of continuous variables (Part 4-1), an analysis of a factor influencing a continuous response variable (Part 4-2), an analysis of association between categorical variables (Part 4-3), a factor analysis and development of a predictive model for categorical response prediction (Part 4-4), a survival data analysis (Part 4-5), and other analyses (Part 4-6).

11. The system of claim 10, wherein each of the Parts for inputting the answer information to each piece of the inquiry information includes example information.

12. The system of claim 1, wherein the conversational interface of the conversational interface means (10) provides selection information for the statistical algorithm selected by the statistical analysis control means (40), and the selection information includes variable and parameter setting information for making a statistical analysis execution stage proceed for the selected statistical algorithm, and further includes item information that enables the user to manually select the statistical analysis.

13. The system of claim 1, wherein the conversational interface that provides selection information for the statistical algorithm selected by the statistical analysis control means (40) and is for selecting a specific statistical algorithm to be used for the analysis includes the following: an upper part in which current program use purpose information is displayed; a lower part inside which the statistical algorithm currently analyzed is displayed; pieces of guide information to be selected/set for setting variables and parameters so that the statistical algorithm proceeds; and a link button for setting variables and parameters, which is a next process according to selection of the user.

14. The system of claim 13, wherein the conversational interface that provides the selection information for the statistical algorithm selected by the statistical analysis control means (40) and is for selecting the specific statistical algorithm to be used for the analysis further includes the following: an automatic/manual selection item for enabling the user to manually select the statistical algorithm; and statistical algorithm information that is selected when manual selection is selected with respect to the selection item.

15. The system of claim 10, wherein among pieces of the inquiry information provided to create the variable characteristic table corresponding to the user's purpose of the statistical analysis by the analysis feature information extraction means (20), the inquiry information provided by a process means for selecting variable characteristics that are a combination of multiple conditions with respect to the analysis of a mean difference between groups of continuous variables (Part 4-1) includes inquiry information on a characteristic of continuous variables, whether a subgroup variable for mean comparison is used, the number of subgroups to be compared, and whether a covariate to be controlled is present.

16. The system of claim 15, wherein regarding the characteristic of continuous variables, included is inquiry information for identifying whether data is independently measured, whether data is constructed in pairs, and whether data is repeatedly measured a predetermined number of times or more with time gaps.

17. The system of claim 10, wherein the statistical algorithms provided from the algorithm management means (30) by the statistical analysis control means (40) with respect to the analysis of a mean difference between groups of continuous variables (Part 4-1) include two-sample T test, paired T test, 1-way ANOVA, repeated measures 1-way ANOVA, repeated measures 2-way ANOVA, 1-way ANCOVA, 1-way ANCOVA with repeated measures, and 2-way ANCOVA with repeated measures.

18. The system of claim 10, wherein a process of selecting the statistical algorithm by the statistical analysis control means (40) with respect to the analysis of a mean difference between groups of continuous variables (Part 4-1) is composed of: a stage of determining a characteristic of continuous variables to identify whether data is independently measured, whether data is constructed in pairs, or whether data is repeatedly measured at least three times with time gaps; a stage of determining whether a subgroup variable is used and determining the number of subgroups to be compared; a stage of determining whether a covariate to be controlled is used; and a stage of selecting the statistical algorithm according to a result of determination through the stage.

19. The system of claim 10, wherein among pieces of the inquiry information provided to create the variable characteristic table corresponding to the user's purpose of the statistical analysis by the analysis feature information extraction means (20), the inquiry information provided by a process means for selecting variable characteristics that are a combination of multiple conditions with respect to the analysis of a factor influencing a continuous response variable (Part 4-2) includes factor variable attribute I including an analysis of individual influence of each independent variable on a response variable, an analysis of influence of two or more independent variables on a response variable, and an analysis of both influence of two categorical variables on a response variable and interaction.

20. The system of claim 10, wherein the statistical algorithms provided from the algorithm management means (30) by the statistical analysis control means (40) with respect to the analysis of a factor influencing a continuous response variable (Part 4-2) include univariable linear regression, multivariable linear regression, and 2-way ANOVA.

21. The system of claim 10, wherein a process of selecting the statistical algorithm by the statistical analysis control means (40) with respect to the analysis of a factor influencing a continuous response variable (Part 4-2) is composed of: a stage of determining a case of an analysis of individual influence of each independent variable on a response variable, a case of an analysis of influence of two or more independent variables on a variable, or a case of an analysis of both influence of two categorical variables on a response variable and interaction; and a stage of selecting the statistical algorithm according to a result of determination at the stage.

22. The system of claim 10, wherein among pieces of the inquiry information provided to create the variable characteristic table corresponding to the user's purpose of the statistical analysis by the analysis feature information extraction means (20), the inquiry information provided by a process means for selecting variable characteristics that are a combination of multiple conditions with respect to the analysis of association between categorical variables (Part 4-3) includes, regarding the analysis of association between categorical variables, the number of categorical variables for which association is to be analyzed, the number of subgroups included in a categorical variable, characteristics of categorical variables, and tendency for proportion of the number of samples in a subgroup, and regarding the number of the categorical variables for which the association is to be analyzed, execution of an analysis of association between two variables, and an analysis of association between three or more variables are included.

23. The system of claim 10, wherein the statistical algorithms provided from the algorithm management means (30) by the statistical analysis control means (40) with respect to the analysis of association between categorical variables (Part 4-3) include Chi-squared test, Yates's correction, Fisher's exact test, linear-by-linear association test, McNemar's test, McNemar-Bowker test, Cochran's Q test with post hoc test, and Friedman test with post hoc analysis.

24. The system of claim 10, wherein a process of selecting the statistical algorithm by the statistical analysis control means (40) with respect to the analysis of association between categorical variables (Part 4-3) is composed of: a stage of performing an analysis of association between two variables or an analysis of three or more variables; and a stage of determining the statistical algorithm by determining a case in which there are two subgroups within a variable or a case in which there are three or more subgroups within a variable, and for the analysis of association between two variables, further included is a stage of selecting the statistical algorithm by determining a case in which data is independently measured or a case in which data is constructed in pairs, and in the case in which there are three or more subgroups within a variable and data is independently measured, further included is a stage of selecting the statistical algorithm by determining a case of a simple association or a case of an analysis of a linear increase/decrease association.

25. The system of claim 24, wherein when the statistical algorithm selected through the stage is Chi-squared test, Yates's correction, or Fisher's exact test, included is a stage of selecting the statistical algorithm by determining a case in which the number of subgroups is four or less or a case in which the number of subgroups is five or more, and in the case in which the number of subgroups are four or less, further included is a stage of determining the statistical algorithm by determining a case in which an expected frequency for a combination of subgroups is four or less or a case in which an expected frequency for a combination of subgroups is five or more.

26. The system of claim 10, wherein among pieces of the inquiry information provided to create the variable characteristic table corresponding to the user's purpose of the statistical analysis by the analysis feature information extraction means (20), the inquiry information provided by a process means for selecting variable characteristics that are a combination of multiple conditions with respect to the factor analysis and development of a predictive model for categorical response prediction (Part 4-4) includes inquiry information about response variable attribute I, response variable attribute II, and a response prediction search type, and
the response variable attribute I includes an item for identifying a response time, the response variable attribute II includes an item for identifying whether there is order between categories included in a response variable, and the response prediction search type includes an item for discovery of a continuous variable and measurement of a cutoff for response prediction, an item for an analysis of individual influence of a variable, an item for development of a predictive model.

27. The system of claim 10, wherein the statistical algorithms provided from the algorithm management means (30) by the statistical analysis control means (40) with respect to the factor analysis and development of a predictive model for categorical response prediction (Part 4-4) include ROC curve analysis (no. cutoff=1), cutoff analysis (no. cutoff>1), univariable binary logistic regression, multivariable binary logistic regression, univariable multinomial logistic regression, multivariable multinomial logistic regression, univariable ordinal logistic regression, multivariable ordinal logistic regression, and logistic mixed effect model analysis.

28. The system of claim 10, wherein a process of selecting the statistical algorithm by the statistical analysis control means (40) with respect to the factor analysis and development of a predictive model for categorical response prediction (Part 4-4) is composed of: a stage of determining a case of a binary response or a case of a ternary or more responses; for the case of the binary response as a result of determination at the stage, a stage of selecting the statistical algorithm through performing discovery of a continuous variable and estimation of a cutoff, an analysis of individual influence of a variable, or development of a predictive model; and for the case of the ternary or more responses, a stage of selecting the statistical algorithm through a stage of determining whether there is order in a response variable category or not and performing, for each of results of determining whether there is order in the response variable category, discovery of a continuous variable and estimation of a cutoff, an analysis of individual influence of a variable, or development of a predictive model.

29. The system of claim 10, wherein among pieces of the inquiry information provided to create the variable characteristic table corresponding to the user's purpose of the statistical analysis by the analysis feature information extraction means (20), the inquiry information provided by a process means for selecting variable characteristics that are a combination of multiple conditions with respect to the survival data analysis (Part 4-5) includes inquiry information of whether there is a competing risk, a prediction parameter attribute, and a survival data research type, and the prediction parameter attribute includes an item for identifying whether an influence on a hazard degree is the same regardless of time, an item for identifying whether there is a change of an influence on a hazard degree over time, and an item for identifying whether an influence on a hazard degree is repeatedly changed over time, and
the survival data research type includes an item for discovery of a continuous prediction parameter and an analysis of a cutoff for survival prediction at a particular time, an item for Kaplan-Meier survival curve analysis, an item for an analysis of individual influence of independent variables for response prediction, and an item for development of a model for response prediction using multiple candidate factors.

30. The system of claim 10, wherein the statistical algorithms provided from the algorithm management means (30) by the statistical analysis control means (40) with respect to the (Part 4-5) include time-dependent ROC curve analysis, Kaplan-Meier curve analysis, univariable Cox proportional hazards regression analysis, multivariable Cox proportional hazards regression analysis, Cox regression using repeatedly measured covariates: univariable analysis, Cox regression using covariates with time-varying effect: multivariable analysis, and Cox regression using repeatedly measured covariates.

31. The system of claim 10, wherein a process of selecting the statistical algorithm by the statistical analysis control means (40) with respect to the survival data analysis (Part 4-5) is composed of: a stage of determining whether there is a competing risk, and for presence and absence of the competing risk each, determining a case of assuming a proportional hazard, a case in which covariates are repeatedly measured over time, or a case in which a covariate has different influences over time; for the case of assuming a proportional hazard, a stage of selecting the statistical algorithm by performing an analysis of a cutoff for particular-time survival prediction, Kaplan-Meier survival curve analysis, an analysis of individual influence of a variable, or development of a predictive model; and for the case in which covariates are repeatedly measured over time or the case in which a covariate has different influence over time, a stage of selecting the statistical algorithm by performing an analysis of individual influence of a variable, or development of a predictive model.

32. The system of claim 10, wherein among pieces of the inquiry information provided to create the variable characteristic table corresponding to the user's purpose of the statistical analysis by the analysis feature information extraction means (20), the inquiry information provided by a process means for selecting variable characteristics that are a combination of multiple conditions with respect to the other analyses (Part 4-6) includes an analysis of correlation between variables (correlation analysis), linear mixed model analysis (linear mixed effect model analysis), logistic mixed model analysis (logistic mixed effect model analysis), Cox mixed model analysis (Cox mixed effect model analysis), a performance comparison analysis of two or more binary diagnosis prediction models (comparison of prediction performance I), a performance comparison analysis of two or more prognosis prediction models (comparison of prediction performance II), cross validation of prediction performance of a binary diagnosis prediction model (cross validation or internal validation I), cross validation of performance of a prognosis prediction model (cross validation or internal validation II), prediction performance validation with data created separately from data used in constructing a prognosis prediction model (external validation I), prediction performance validation with data created separately from data used in constructing a prognosis prediction model (external validation II), and test for difference between proportions of the number of samples in two or more subgroups (one-sample proportion test).

33. A statistical analysis method using a conversational interface, the method comprising:
a feature information extraction induction stage in which when a statistical analysis program starts, the conversational interface for conversation with a user is provided so that inquiries are made to the user and answer information to the inquiries is acquired and stored; an analysis feature information extraction stage in which after the feature information extraction induction stage, analysis feature information for selecting a statistical algorithm is extracted from the answer information; an algorithm setting stage in which the algorithm matched to the analysis feature information extracted at the analysis feature information extraction stage is set as a statistical analysis algorithm; a statistical analysis execution stage in which a statistical analysis is performed according to the algorithm set through the algorithm setting stage; and a statistical analysis result providing stage in which the user is provided with results acquired through the statistical analysis execution stage.

34. (canceled)

35. The method of claim 33, wherein the feature information extraction induction stage comprises: a purpose inquiry stage in which when a statistical analysis system starts, a request to start a conversation analysis is made, and when a request to start the conversation analysis is made from the user, the user is provided with an inquiry for acquiring a purpose of use of the program and an answer thereto is input; and a variable characteristic information inquiry stage in which when the answer is input through the purpose inquiry stage, the answer is stored in an analysis feature information extraction means, inquiry information is provided to create a variable characteristic table corresponding to the user's selected purpose of the statistical analysis, and the answer information thereto is input and stored as the analysis feature information.

36. (canceled)

37. The method of claim 33, wherein at the feature information extraction induction stage, the inquiry information provided to the user to acquire the purpose of the statistical analysis includes at least any one among an analysis required for making a research plan, sub-data extraction for a research from raw data and merging of different pieces of data, clinical data preprocessing for a statistical analysis, a statistical analysis using clinical data, a meta-analysis using an existing analysis result, and a reliability analysis of acquired data.

38. The method of claim 37, wherein when at the feature information extraction induction stage, among pieces of the inquiry information, the statistical analysis using clinical data (Part 4) is selected, the inquiry information provided to create the variable characteristic table corresponding to the user's purpose of the statistical analysis includes an analysis of a mean difference between groups of continuous variables (Part 4-1), an analysis of a factor influencing a continuous response variable (Part 4-2), an analysis of association between categorical variables (Part 4-3), a factor analysis and development of a predictive model for categorical response prediction (Part 4-4), a survival data analysis (Part 4-5), and other analyses (Part 4-6).

39-40. (canceled)
